(19) United States
(12) Patent Application Publication
(10) Pub. No.: US 2024/0139377 A1
(43) Pub. Date: May 2, 2024

(54) Preformed Neural Tissue to Restore or Augment Auditory Inputs to the Brain

(71) Applicants: THE TRUSTEES OF THE UNIVERSITY OF PENNSYLVANIA, Philadelphia, PA (US); The United States Government as represented by the Department of Veterans Affairs, Washington, DC (US)

(72) Inventors: Daniel Kacy Cullen, Media, PA (US); Jason Brant, Wallingford, PA (US); Oladayo Adewole, Philadelphia, PA (US)

(21) Appl. No.: 18/277,046

(22) PCT Filed: Feb. 23, 2022
(86) PCT No.: PCT/US22/17470

§ 371 Date: Aug. 11, 2023

Related U.S. Application Data
(60) Provisional application No. 63/153,321, filed on Feb. 24, 2021.
(60) Provisional application No. 63/234,048, filed on Aug. 17, 2021.

(51) Int. Cl. A61L 27/38; A61L 27/26; A61L 27/52; A61N 1/05; A61N 5/06

(52) U.S. Cl. CPC A61L 27/383; A61L 27/26; A61L 27/3834; A61L 27/3878; A61L 27/3886; A61L 27/52; A61N 1/0541; A61N 5/0603; A61N 5/0622; A61L 2430/14; A61N 2005/0605

(57) ABSTRACT

Provided herein is a system, e.g., a living electrode, comprising a biocompatible construct comprising a matrix, and a plurality of auditory neurons. Also disclosed herein are methods of making the system, and methods of using the same for implantation in a subject, for modulating an auditory neuron in the subject, and/or for treating or alleviating a symptom of a hearing loss disorder. Further provided herein are kits comprising the system described herein.

### CROSS-REFERENCE TO RELATED APPLICATIONS

The present application claims priority under 35 U.S.C. § 119(e) to U.S. Provisional Patent Applications No. 63/153,321, filed Feb. 24, 2021, and No. 63/234,048, filed Aug. 17, 2021, all of which are incorporated herein by reference in their entireties.

### STATEMENT REGARDING FEDERALLY SPONSORED RESEARCH OR DEVELOPMENT

This invention was made with government support under NS094340 awarded by the National Institutes of Health. The government has certain rights in the invention.

### BACKGROUND

Hearing loss disorders affect a large proportion of the world's population. One of the treatment options for patients with hearing loss disorders is an auditory implant. However, the implants that are currently available have several limitations including an inability to restore a patient's natural hearing, morbidity upon implantation, and difficulty understanding of complex sounds.

### SUMMARY

Provided herein, inter alia, is a system, e.g., a living electrode, comprising a biocompatible construct comprising a matrix, and a plurality of auditory neurons. Also disclosed herein are methods of making a system, e.g., comprising at least one living electrode, comprising a biocompatible construct comprising a matrix, and a plurality of auditory neurons, and methods of using the same for implantation in a subject, for modulating an auditory neuron in a subject, and/or for treating or alleviating a symptom of a hearing loss disorder. Further provided herein are kits comprising a system described herein.

The present disclosure provides a system, e.g., comprising at least one living electrode, comprising: (a) a biocompatible construct comprising a matrix comprising a first end, a second end, and a body; and (b) a plurality of auditory neurons (e.g., vestibular neurons and/or cochlear neurons).

In some embodiments, a biocompatible construct comprises an inner surface and/or an outer surface.

In some embodiments, an inner surface of a biocompatible construct defines a luminal core.

In some embodiments, an outer surface of a biocompatible construct comprises at least one hydrogel. In some embodiments, a hydrogel comprises a hydrophilic biopolymer and/or a synthetic polymer. In some embodiments, a hydrogel is at least partially cross-linked, wherein the cross-linking optionally increases stiffness, reduces porosity, and/or increases degradation time. In some embodiments, a hydrophilic biopolymer comprises one or more of agarose, hydrogel, hyaluronan, chitosan, alginate, collagen, dextran, pectin, carrageenan, polylysine, gelatin, hyaluronic acid, fibrin, and methylcellulose. In some embodiments, a hydrophilic biopolymer comprises agarose. In some embodiments, a hydrophilic biopolymer comprises hydrogel. In some embodiments, a hydrophilic biopolymer comprises chitosan. In some embodiments, a hydrophilic biopolymer comprises alginate. In some embodiments, a hydrophilic biopolymer comprises collagen. In some embodiments, a hydrophilic biopolymer comprises dextran. In some embodiments, a hydrophilic biopolymer comprises pectin. In some embodiments, a hydrophilic biopolymer comprises carrageenan. In some embodiments, a hydrophilic biopolymer comprises polylysine. In some embodiments, a hydrophilic biopolymer comprises gelatin. In some embodiments, a hydrophilic biopolymer comprises hyaluronic acid. In some embodiments, a hydrophilic biopolymer comprises fibrin. In some embodiments, a hydrophilic biopolymer comprises methylcellulose.

In some embodiments, a hydrophilic biopolymer comprises agarose. In some embodiments, an agarose is at about 0.25-30%, about 0.25%-3%, about 0.5%-3%, about 1-20%, about 1.5-10%, about 2-9%, about 2.5-8%, or about 3-7%. In some embodiments, the agarose is at about 0.25-29%, about 0.25-28%, 0.25-%, about 0.25-27%, about 0.25-26%, about 0.25-25%, about 0.25-24%, about 0.25-23%, about 0.25-22%, about 0.25-21%, about 0.25-20%, about 0.25-19%, about 0.25-18%, about 0.25-17%, about 0.25-16%, about 0.25-15%, about 0.25-14%, about 0.25-13%, about 0.25-12%, about 0.25-11%, about 0.25-10%, about 0.25-9%, about 0.25-8%, about 0.25-7%, about 0.25-6%, about 0.25-5%, about 0.25-4%, about 0.25-3%, about 0.25-2%, about 0.25-1%, about 0.25-0.5%, about 0.5-30%, about 1-30%, about 2-30%, about 3-30%, about 4-30%, about 5-30%, about 6-30%, about 7-30%, about 8-30%, about 9-30%, about 10-30%, about 11-30%, about 12-30%, about 13-30%, about 14-30%, about 15-30%, about 16-30%, about 17-30%, about 18-30%, about 19-30%, about 20-30%, about 21-30%, about 22-30%, about 23-30%, about 24-30%, about 25-30%, about 26-30%, about 27-30%, about 28-30%, about 29-30%.

In some embodiments, an agarose is at about 0.25%, about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%. In some embodiments, an agarose is at about 3%.

In some embodiments, a hydrophilic biopolymer comprises at least one synthetic hydrogel (e.g., Pluronic hydrogel) or at least one synthetic hydrogel made of amphiphilic copolymers consisting of units of ethylene oxide (PEO) and polypropylene oxide (PPO). In some embodiments, a hydrogel is at about 2-50% w/v, about 3-40% w/v, about 4-30% w/v, about 5-25% w/v or about 10-20% w/v. In some embodiments, a hydrogel is at about 2-45%, about 2-40%, about 2-35%, about 2-30%, about 2-29%, about 2-28%, about 2-27%, about 2-26%, about 2-25%, about 2-24%, about 2-23%, about 2-22%, about 2-21%, about 2-20%, about 2-19%, about 2-18%, about 2-17%, about 2-16%, about 2-15%, about 2-14%, about 2-13%, about 2-12%, about 2-11%, about 2-10%, about 2-9%, about 2-8%, about 2-7%, about 2-6%, about 2-5%, about 2-4%, about 2-3%, about 3-50%, about 4-50%, about 5-50%, about 6-50%, about 7-50%, about 8-50%, about 9-50%, about 10-50%, about 11-50%, about 12-50%, about 13-50%, about 14-50%, about 15-50%, about 16-50%, about 17-50%, about 18-50%, about 19-50%, about 20-50%, about 21-50%, about 22-50%, about 23-50%, about 24-50%, about 25-50%, about 26-50%, about 27-50%, about 28-50%, about 29-50%, about 30-50%, about 35-50%, about 40-50%, about 45-50% w/v. In some embodiments, a hydrogel is at about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 40%, about 45%, about 50% w/v. In some embodiments, a hydrogel is at about 28.1%, about 28.2%, about 28.3%, about 28.4%, about 28.5%, about 28.6%, about 28.7%, about 28.8%, about 28.9%, about 30% w/v. In some embodiments, a hydrogel is at about 28.6% w/v.

In some embodiments, an inner surface of a biocompatible construct comprises one or more extracellular matrix (ECM) components. In some embodiments, an ECM component comprises collagen, laminin, fibronectin, hyaluronic acid, or a combination thereof. In some embodiments, an ECM component comprises collagen. In some embodiments, an ECM component comprises laminin. In some embodiments, an ECM component comprises fibronectin. In some embodiments, an ECM component comprises hyaluronic acid.

In some embodiments, an ECM component comprises collagen. In some embodiments, a collagen is at a concentration of about 0.1-10 mg/ml. In some embodiments, a collagen is at a concentration of about 0.1-9 mg/ml, about 0.1-8 mg/ml, about 0.1-7 mg/ml, about 0.1-6 mg/ml, about 0.1-5 mg/ml, about 0.1-4 mg/ml, about 0.1-3 mg/ml, about 0.1-2 mg/ml, about 0.1-1 mg/ml, about 0.1-0.9 mg/ml, about 0.1-0.8 mg/ml, about 0.1-0.7 mg/ml, about 0.1-0.5 mg/ml, about 0.5-10 mg/ml, about 0.6-10 mg/ml, about 0.7-10 mg/ml, about 0.8-10 mg/ml, about 0.9-10 mg/ml, about 1-10 mg/ml, about 2-10 mg/ml, about 3-10 mg/ml, about 4-10 mg/ml, about 5-10 mg/ml, about 6-10 mg/ml, about 7-10 mg/ml, about 8-10 mg/ml, about 9-10 mg/ml. In some embodiments, a collagen is at a concentration of about 0.1 mg/ml, about 0.2 mg/ml, about 0.3 mg/ml, about 0.4 mg/ml, about 0.5 mg/ml, about 0.6 mg/ml, about 0.7 mg/ml, about 0.8 mg/ml, about 0.9 mg/ml, about 1 mg/ml, about 1.1 mg/ml, about 1.2 mg/ml, about 1.3 mg/ml, about 1.4 mg/ml, about 1.5 mg/ml, about 1.6 mg/ml, about 1.7 mg/ml, about 1.8 mg/ml, about 1.9 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml. In some embodiments, the collagen is at a concentration of about 1 mg/ml.

In some embodiments, an ECM component comprises laminin. In some embodiments, a laminin is at a concentration of about 0.1-9 mg/ml, about 0.1-8 mg/ml, about 0.1-7 mg/ml, about 0.1-6 mg/ml, about 0.1-5 mg/ml, about 0.1-4 mg/ml, about 0.1-3 mg/ml, about 0.1-2 mg/ml, about 0.1-1 mg/ml, about 0.1-0.9 mg/ml, about 0.1-0.8 mg/ml, about 0.1-0.7 mg/ml, about 0.1-0.5 mg/ml, about 0.5-10 mg/ml, about 0.6-10 mg/ml, about 0.7-10 mg/ml, about 0.8-10 mg/ml, about 0.9-10 mg/ml, about 1-10 mg/ml, about 2-10 mg/ml, about 3-10 mg/ml, about 4-10 mg/ml, about 5-10 mg/ml, about 6-10 mg/ml, about 7-10 mg/ml, about 8-10 mg/ml, about 9-10 mg/ml. In some embodiments, a laminin is at a concentration of about 0.1 mg/ml, about 0.2 mg/ml, about 0.3 mg/ml, about 0.4 mg/ml, about 0.5 mg/ml, about 0.6 mg/ml, about 0.7 mg/ml, about 0.8 mg/ml, about 0.9 mg/ml, about 1 mg/ml, about 1.1 mg/ml, about 1.2 mg/ml, about 1.3 mg/ml, about 1.4 mg/ml, about 1.5 mg/ml, about 1.6 mg/ml, about 1.7 mg/ml, about 1.8 mg/ml, about 1.9 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml. In some embodiments, a laminin is at a concentration of about 1 mg/ml.

In some embodiments, a plurality of auditory neurons comprises about 50-5000 auditory neurons (e.g., auditory neurons described herein, e.g., vestibular neurons and/or cochlear neurons). In some embodiments, a plurality of auditory neurons comprises about 50-4500, about 50-4000, about 50-3500, about 50-3000, about 50-2500, about 50-2000, about 50-1500, about 50-1000, about 50-900, about 50-800, about 50-700, about 50-600, about 50-550, about 50-500, about 50-450, about 50-400, about 50-300, about 50-200, about 50-100, about 100-5000, about 200-5000, about 300-5000, about 400-5000, about 500-5000, about 1000-5000, about 2000-5000, about 3000-5000 or about 4000-5000 auditory neurons. In some embodiments, a plurality of auditory neurons comprises at least 50 auditory neurons, at least 100 auditory neurons, at least 200 auditory neurons, at least 300 auditory neurons, at least 400 auditory neurons, at least 450 auditory neurons, at least 500 auditory neurons, at least 550 auditory neurons, at least 600 auditory neurons, at least 700 auditory neurons, at least 800 auditory neurons, at least 900 auditory neurons, at least 1000 auditory neurons, at least 2000 auditory neurons, at least 3000 auditory neurons, at least 4000, or at least 5000 auditory neurons. In some embodiments, a plurality of auditory neurons comprises at least 500 auditory neurons.

In some embodiments, an auditory neuron is or comprises a neuron having one or more characteristics of a neuron present in a cochlea, spiral ganglion, auditory nerve, auditory brainstem nuclei, and/or cortical brainstem nuclei of an organism, e.g., a human. In some embodiments, an auditory neuron is or comprises a neuron derived from a neuron present in a cochlea, spiral ganglion, auditory nerve, auditory brainstem nuclei, and/or cortical brainstem nuclei of an organism (e.g., a human) or a combination thereof.

In some embodiments, at least one of a plurality of auditory neurons is in contact with a biocompatible construct.

In some embodiments, at least one neuron in a plurality of neurons comprises a cell body and/or an axon. In some embodiments, at least one neuron in a plurality of neurons comprises a cell body. In some embodiments, at least one neuron in a plurality of neurons comprises an axon. In some embodiments, at least one neuron in a plurality of neurons comprises a cell body and an axon. In some embodiments, an axon is about 0.5-50 cm in length.

In some embodiments, a portion of at least one neuron in the plurality of auditory neurons is positioned: (i) at a first end of the biocompatible construct; (ii) at a second end of the biocompatible construct; (iii) at, along or within a portion of a body of the biocompatible construct; and/or (iv) at, along, or within the entire body of the biocompatible construct. In some embodiments, a portion of at least one neuron in the plurality of auditory neurons is positioned at a first end of the biocompatible construct. In some embodiments, a portion of at least one neuron in the plurality of auditory neurons is positioned at a second end of the biocompatible construct.

In some embodiments, a portion of at least one neuron in the plurality of auditory neurons is positioned at, along or within a portion of a body of the biocompatible construct. In some embodiments, a portion of at least one neuron in the plurality of auditory neurons is positioned at, along, or within the entire body of the biocompatible construct.

In some embodiments, a portion of at least one neuron in the plurality of auditory neuron positioned at a first end or at a second end of the biocompatible construct comprises a cell body.

In some embodiments, a portion of at least one neuron in the plurality of auditory neuron positioned at, along or within a portion of a body of the biocompatible construct comprises an axon. In some embodiments, a body of the biocompatible construct comprises an inner surface of the body. In some embodiments, an inner surface comprises a luminal core.

In some embodiments, a portion of at least one neuron in the plurality of auditory neuron positioned at, along or within the entire body of the biocompatible construct comprises an axon. In some embodiments, the axon extends unidirectionally or bidirectionally. In some embodiments, the axon extends unidirectionally. In some embodiments, the axon extends bidirectionally.

In some embodiments, at least a portion of the plurality of auditory neurons comprises an aggregate of auditory neurons.

In some embodiments, a plurality of auditory neurons comprises a spiral ganglion neuron, an inferior colliculus (IC) auditory neuron, a cortical auditory neuron, a type I auditory neuron, or a type II auditory neuron. In some embodiments, a plurality of auditory neurons is or comprises a plurality of spiral ganglion neurons. In some embodiments, a plurality of auditory neurons is or comprises a plurality of IC auditory neurons. In some embodiments, a plurality of auditory neurons is or comprises a plurality of cortical auditory neurons. In some embodiments, a plurality of auditory neurons is or comprises a plurality of type I auditory neurons. In some embodiments, a plurality of auditory neurons is or comprises a plurality of type II auditory neurons.

In some embodiments, a system disclosed herein, e.g., a living electrode, further comprises one or more additional cells, such as astrocytes, oligodendrocytes, Schwann cells, microglia, or endothelial cells.

In some embodiments, a plurality of auditory neurons is isolated from a subject.

In some embodiments, a plurality of auditory neurons is or comprises at least one cell derived from a stem cell. In some embodiments, a stem cell comprises one or more of an induced pluripotent stem cell (iPSC), a fetal stem cell, or a tissue stem cell. In some embodiments, a stem cell comprises an iPSC. In some embodiments, a stem cell comprises a fetal stem cell. In some embodiments, a stem cell comprises a tissue stem cell.

In some embodiments, a plurality of auditory neurons is autologous, allogeneic, or xenogeneic to a subject. In some embodiments, a plurality of auditory neurons is autologous to a subject. In some embodiments, a plurality of auditory neurons is allogeneic to a subject. In some embodiments, a plurality of auditory neurons is xenogeneic to a subject.

In some embodiments, at least one neuron in the plurality of auditory neurons is genetically engineered or reprogrammed using a non-genetic technique. In some embodiments, at least one genetically engineered neuron comprises a sensor, an actuator, a receptor and/or a reporter.

In some embodiments, at least one genetically engineered neuron in the plurality of auditory neurons comprises a reporter. In some embodiments, the reporter comprises a fluorescent reporter.

In some embodiments, at least one genetically engineered neuron in the plurality of auditory neurons comprises a receptor. In some embodiments, the receptor comprises one or more of a magnetoreceptor, an electromagnetic receptor, an electroreceptor, a hydroreceptor, a mechanoreceptor, an osmoreceptor, a thermoreceptor, and a piezoelectric ion channel.

In some embodiments, an electromagnetic receptor comprises one or more of an infrared receptors, a photoreceptor, or an ultraviolet receptors.

In some embodiments, a mechanoreceptor comprises one or both of a mechanosensory receptor, or a proprioreceptor.

In some embodiments, at least one genetically engineered neuron in the plurality of auditory neurons comprises a sensor. In some embodiments, the sensor comprises an optical sensor comprising one or more of a light-sensitive ion channel, a calcium sensor, and a membrane voltage sensor.

In some embodiments, a sensor comprises a calcium sensor comprising one or more of Aequorin, Cameleon, and GCaMP.

In some embodiments, a sensor comprises a chloride sensor comprising Clomeleon.

In some embodiments, a sensor comprises a membrane voltage sensor comprising Mermaid.

In some embodiments, at least one genetically engineered neuron in the plurality of auditory neurons comprises an actuator. In some embodiments, the actuator comprises an optical actuator comprising one or more of channelrhodopsin, halorhodopsin, and archaerhodopsin.

In some embodiments, at least one genetically engineered neuron in the plurality of auditory neurons comprising an optical sensor and/or an optical actuator is an optogenetic neuron.

In some embodiments, a biocompatible construct is a tubular construct. In some embodiments, a biocompatible construct is a spiral or a wye. In some embodiments, a biocompatible construct has a curvature of about 240-900 degrees. In some embodiments, the curvature of the biocompatible construct is about 270 degrees.

In some embodiments, a system, e.g., comprising at least one living electrode, has an outer diameter of about 0.1-40 mm. In some embodiments, a system has an outer diameter of about 0.1-35 mm, about 0.1-30 mm, about 0.1-25 mm, about 0.1-20 mm, about 0.1-15 mm, about 0.1-12 mm, about 0.1-11 mm, about 0.1-10 mm, about 0.1-9 mm, about 0.1-8 mm, about 0.1-7 mm, about 0.1-6.5 mm, about 0.1-6 mm, about 0.1-5 mm, about 0.1-4 mm, about 0.1-3 mm, about 0.1-2 mm, about 0.1-1 mm, about 0.1-0.5 mm, about 0.2-40 mm, about 0.3-40 mm, about 0.4-40 mm, about 0.5-40 mm, about 1-40 mm, about 2-40 mm, about 3-40 mm, about 4-40 mm, about 5-40 mm, about 6-40 mm, about 7-40 mm, about 8-40 mm, about 9-40 mm, about 10-40 mm, about 11-40 mm, about 12-40 mm, about 15-40 mm, about 20-40 mm, about 25-40 mm, about 30-40 mm, or about 35-40 mm. In some embodiments, a system has an outer diameter of about 0.1 mm, about 0.5 mm, about 1 mm, about 2 mm, about 3 mm, about 4 mm, about 5 mm, about 6 mm, about 6.5 mm, about 7 mm, about 8 mm, about 9 mm, about 10 mm, about 11 mm, about 12 mm, about 13 mm, about 14 mm, about 15 mm, about 20 mm, about 25 mm, about 30 mm, about 35 mm or about 40 mm. In some embodiments, a system has an outer diameter of about 1.5 mm.

In some embodiments, a system, e.g., comprising at least one living electrode, has a diameter of about 0.5-70 mm, about 0.5-60 mm, about 0.5-50 mm, about 0.4-50 mm, about 0.5-30 mm, about 0.5-20 mm, about 0.5-10 mm, about 1-70 mm, about 2-70 mm, about 3-70 mm, about 4-70 mm, about 5-70 mm, about 6-70 mm, about 7-70 mm, about 8-70 mm, about 9-70 mm about 10-70 mm, about 20-70 mm, about 30-70 mm about 40-70 mm about 50-70 mm. In some embodiments, the system has a diameter of about 0.1-10 mm, about 0.2-9 mm, about 0.3-8 mm, about 0.4-7 mm, about 0.5-6.5 mm. In some embodiments, a system has a diameter of about 0.5 mm, about 1 mm, about 2 mm, about 3 mm, about 4 mm, about 5 mm, about 6 mm, about 6.5 mm, about 7 mm, about 8 mm, about 9 mm, about 10 mm, about 20 mm, about 30 mm, about 40 mm, about 50 mm, about 60 mm, about 70 mm. In some embodiments, a system has a diameter of about 7 mm.

In some embodiments, a system, e.g., comprising at least one living electrode, has a pitch of about 0.1-10 mm, about 0.1-8 mm, about 0.1-6 mm, about 0.1-4 mm, about 0.1-3 mm, about 0.1-2 mm, about 0.1-1.5 mm, about 0.1-1 mm, about 0.5-10 mm, about 0.6-10 mm, about 0.7-10 mm, about 0.8-10 mm about 0.9-10 mm, about 1-10 mm, about 2-10 mm, about 4-10 mm, about 6-10 mm about 8-10 mm. In some embodiments, a system has a pitch of about 0.1 mm, about 0.2 mm, about 0.3 mm, about 0.4 mm, about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 3 mm, about 4 mm, about 5 mm, about 6 mm, about 7 mm, about 8 mm, about 9 mm, about 10 mm. In some embodiments, a system has a pitch of about 1 mm.

In some embodiments, a system, e.g., comprising at least one living electrode, is manufactured, e.g., assembled, ex vivo or in vitro. In some embodiments, a system is manufactured, e.g., assembled, ex vivo. In some embodiments, a system is manufactured, e.g., assembled, in vitro.

In some embodiments, a system, e.g., comprising at least one living electrode, is capable of, or can be used to, modulate and/or assess at least one activity of at least one neuron in the plurality of neurons in the system and/or at least one neuron in a subject. In some embodiments, a system is capable of, or can be used to, modulate at least one activity of at least one neuron in the plurality of neurons in the system. In some embodiments, a system is capable of, or can be used to, modulate at least one activity of at least one neuron in a subject. In some embodiments, a system is capable of, or can be used to, modulate at least one activity of at least one neuron in the plurality of neurons in the system and at least one neuron in a subject.

In some embodiments, modulating is or comprises unidirectional or bidirectional modulation. In some embodiments, modulating is or comprises unidirectional modulation. In some embodiments, modulating is or comprises bidirectional modulation.

In some embodiments, modulating comprises stimulating or inhibiting at least one activity of at least one neuron in the plurality of auditory neurons in the system and/or at least one neuron in a subject. In some embodiments, modulating comprises stimulating or inhibiting at least one activity of at least one neuron in the plurality of auditory neurons in the system. In some embodiments, modulating comprises stimulating or inhibiting at least one activity of at least one neuron in a subject. In some embodiments, modulating comprises stimulating or inhibiting at least one activity of at least one neuron in the plurality of auditory neurons in the system and at least one neuron in a subject.

In some embodiments, stimulating comprises electrical stimulation; optical stimulation; sound wave or vibration stimulation; magnetic stimulation; or a combination thereof. In some embodiments, stimulating comprises electrical stimulation. In some embodiments, stimulating comprises optical stimulation. In some embodiments, stimulating comprises sound wave or vibration stimulation. In some embodiments, stimulating comprises magnetic stimulation. In some embodiments, stimulating comprises electrical stimulation and optical stimulation.

In some embodiments, assessing comprises monitoring and/or recording. In some embodiments, assessing comprises monitoring. In some embodiments, assessing comprises recording. In some embodiments, assessing comprises monitoring and recording.

In some embodiments, at least one neuron in a subject is or comprises an auditory neuron. In some embodiments, an auditory neuron comprises a vestibular neuron, a cochlear neuron, spiral ganglion neuron, an IC neuron, or a combination thereof.

In some embodiments, a system, e.g., comprising at least one living electrode, is capable of stimulating or inhibiting at least one activity of at least one auditory pathway in a subject. In some embodiments, a system is capable of stimulating at least one activity of at least one auditory pathway in a subject. In some embodiments, a system is capable of inhibiting at least one activity of at least one auditory pathway in a subject.

In some embodiments, a system, e.g., comprising at least one living electrode, is capable of stimulating or inhibiting at least one activity of at least one peripheral auditory pathway in a subject. In some embodiments, a system is capable of stimulating at least one activity of at least one peripheral auditory pathway in a subject. In some embodiments, a system is capable of inhibiting at least one activity of at least one peripheral auditory pathway in a subject.

In some embodiments, a system disclosed herein, e.g., comprising at least one living electrode, is implanted at, near, or within a cochlea in a subject, at, near, or within an inferior colliculus (IC) in a subject, at, near, or within a cochlear nucleus in a subject, and/or at, near or within an auditory cortex in a subject. In some embodiments, a system is implanted at, near, or within a cochlea in a subject. In some embodiments, a system is implanted at a cochlea in a subject. In some embodiments, a system is implanted near a cochlea in a subject. In some embodiments, a system is implanted within a cochlea in a subject. In some embodiments, a system is implanted at, near, or within an inferior colliculus (IC) in a subject. In some embodiments, a system is implanted at an inferior colliculus (IC) in a subject. In some embodiments, a system is implanted near an inferior colliculus (IC) in a subject. In some embodiments, a system is implanted within an inferior colliculus (IC) in a subject. In some embodiments, a system is implanted at, near, or within a cochlear nucleus in a subject. In some embodiments, a system is implanted at a cochlear nucleus in a subject. In some embodiments, a system is implanted near a cochlear nucleus in a subject. In some embodiments, a system is implanted within a cochlear nucleus in a subject. In some embodiments, a system is implanted at, near, or within an auditory cortex in a subject. In some embodiments, a system is implanted at an auditory cortex in a subject. In some embodiments, a system is implanted near an auditory cortex in a subject. In some embodiments, a system is implanted within an auditory cortex in a subject.

In some embodiments, a plurality of systems disclosed herein, e.g., comprising at least one living electrode, are implanted at, near, or within a cochlea in a subject, at, near, or within an inferior colliculus (IC) in a subject; at, near or within a cochlear nucleus in a subject, and/or at, near or within an auditory cortex in a subject. In some embodiments, a first system disclosed herein and a second system disclosed herein are implanted in a subject. In some embodiments, a first system disclosed herein and a second system disclosed herein are the same, e.g., two similar systems disclosed herein are implanted in a subject. In some embodiments, a first system disclosed herein and a second system disclosed herein are different, e.g., two different systems disclosed herein are implanted in a subject. In some embodiments, a third or further (e.g., fourth, fifth, sixth, seventh, eighth, ninth, tenth, etc.) system disclosed herein is implanted in a subject.

In some embodiments, a first system is implanted at, near, or within a cochlea in a subject. In some embodiments, a first system is implanted at a cochlea in a subject. In some embodiments, a system is implanted near a cochlea in a subject. In some embodiments, a first system is implanted within a cochlea in a subject. In some embodiments, a second system is implanted at, near, or within an inferior colliculus (IC) in a subject. In some embodiments, a second system is implanted at an inferior colliculus (IC) in a subject. In some embodiments, a second system is implanted near an inferior colliculus (IC) in a subject. In some embodiments, a system is implanted within an inferior colliculus (IC) in a subject.

In some embodiments, a second system is implanted at, near, or within a cochlear nucleus in a subject. In some embodiments, a second system is implanted at a cochlear nucleus in a subject. In some embodiments, a second system is implanted near a cochlear nucleus in a subject. In some embodiments, a system is implanted within a cochlear nucleus in a subject. In some embodiments, a second system is implanted at, near, or within an auditory cortex in a subject. In some embodiments, a second system is implanted at an auditory cortex in a subject. In some embodiments, a second system is implanted near an auditory cortex in a subject. In some embodiments, a system is implanted within an auditory cortex in a subject.

In some embodiments, an implanted system or a plurality of implanted systems disclosed herein contacts at least one cell in a subject or at, near, or within areas of the auditory cortex in a subject. In some embodiments, at least one cell in a subject is an endogenous cell. In some embodiments, at least one cell comprises a neuron. In some embodiments, a neuron comprises an auditory neuron, e.g., as described herein. In some embodiments, an auditory neuron comprises a neuron having one or more characteristics of a neuron present in a cochlea, spiral ganglion, auditory nerve, auditory brainstem nuclei, and/or cortical brainstem nuclei of an organism (e.g., a human), or a combination thereof. In some embodiments, an auditory neuron comprises one or more of a spiral ganglion neuron or an IC neuron.

In some embodiments, contacting of a system or a plurality of systems disclosed herein with at least one cell in a subject is determined to create a synapse between at least one neuron of the plurality of auditory neurons of the system and at least one cell in a subject. In some embodiments, a synapse is or comprises a neuronal synapse.

In some embodiments, an implanted system or a plurality of implanted systems disclosed herein provides an accessible interface for modulating at least one neuron in the plurality of auditory neurons in the system, and/or at least one cell in a subject. In some embodiments, an implanted system or a plurality of implanted systems disclosed herein provides an accessible interface for modulating at least one neuron in the plurality of auditory neurons in the system. In some embodiments, an implanted system or a plurality of implanted systems disclosed herein provides an accessible interface for modulating at least one cell in the subject.

In some embodiments, a biocompatible construct has a non-linear shape.

In some embodiments, a system disclosed herein further comprises a stimulator array. In some embodiments, a stimulator array is or comprises an optrode array.

Provided herein is a system, e.g., a cochlear implant, comprising:

a stimulator array;

a plurality of living electrodes, wherein at least one of the plurality of living electrodes is in optical and/or electrical communication with an element of the stimulator array, each of the plurality of living electrodes comprising: (1) a proximal end comprising stimulator-complementary neurons; and (2) a distal end comprising auditory neurons in neurologic communication with optogenetic neurons within the living electrode;

wherein the plurality of living electrodes have staggered distal ends.

In some embodiments, a stimulator array is or comprises an optrode array.

In some embodiments, stimulator-complementary neurons are or comprise optogenetic neurons.

Also provided herein is a method for manufacturing a system, e.g., comprising at least one living electrode, comprising: (a) providing a biocompatible construct comprising a matrix comprising a first end, a second end, and a body; and (b) associating the biocompatible construct with a plurality of auditory neurons (e.g., vestibular neurons and/or cochlear neurons).

In some embodiments, a method described herein comprises maintaining a system under conditions that promotes growth of at least one neuron in a plurality of auditory neurons. In some embodiments, a method described herein comprises maintaining a system under conditions that maintain viability of at least one neuron in the plurality of auditory neurons.

In some embodiments, a method described herein further comprises forming an aggregate of at least a portion of the plurality of auditory neurons. In some embodiments, an aggregate of at least a portion of the plurality of auditory neurons is formed prior to contacting the plurality of auditory neurons with a biocompatible construct.

This disclosure provides a method for implanting in a subject, a system, e.g., comprising at least one living electrode, comprising: (a) a biocompatible construct comprising a matrix comprising a first end, a second end, and a body; and (b) a plurality of auditory neurons (e.g., vestibular neurons and/or cochlear neurons), the method comprising implanting the system at, near, or within a cochlea, an inferior colliculus (IC), a cochlear nucleus or an auditory cortex in a subject.

In some embodiments, an implantation is determined to be less likely to cause inflammation compared to implantation of conventional microelectrodes.

In some embodiments of any of the methods disclosed herein, a subject is a human.

Provided herein is a method of modulating and/or assessing at least one activity of at least one neuron in a subject, comprising: (i) providing to the subject, a system, e.g., comprising at least one living electrode, comprising: (a) a biocompatible construct comprising a matrix comprising a first end, a second end, and a body; and (b) a plurality of auditory neurons (e.g., vestibular neurons and/or cochlear neurons), and (ii) modulating at least one neuron with electrical stimulation, optical stimulation or both.

In some embodiments, assessing comprises monitoring, recording, stimulating and/or inhibiting at least one neuron in a subject. In some embodiments, assessing comprises monitoring at least one neuron in a subject. In some embodiments, assessing comprises recording at least one neuron in a subject. In some embodiments, assessing comprises stimulating at least one neuron in a subject. In some embodiments, assessing comprises inhibiting at least one neuron in a subject.

In some embodiments, at least one neuron in a subject comprises an auditory neuron.

In some embodiments, an auditory neuron comprises a neuron having one or more characteristics of a neuron present in a cochlea, spiral ganglion, auditory nerve, auditory brainstem nuclei, a cortical brainstem nuclei, or a combination thereof. In some embodiments, an auditory neuron comprises one or more of a vestibular neuron, a cochlear neuron, spiral ganglion neuron, or an IC neuron.

In some embodiments of any of the methods disclosed herein, a subject is a human.

This disclosure further provides a method of treating a subject having a hearing loss disorder, comprising providing to the subject a system, e.g., comprising at least one living electrode, comprising: (a) a biocompatible construct comprising a matrix comprising a first end, a second end, and a body; and (b) a plurality of auditory neurons, thereby treating the subject.

In some embodiments, a hearing loss disorder comprises one or more of conductive hearing loss, sensorineural hearing loss or mixed hearing loss. In some embodiments, a hearing loss disorder comprises one or more of acquired hearing loss disorder, sudden hearing loss disorder, secondary hearing loss (drug, noise, or injury induce hearing loss), autoimmune inner ear disease, otosclerosis, Meniere's disease, presbycusis, or acoustic neuroma.

In some embodiments of any of the methods disclosed herein, a subject is a human.

Provided herein is a method of ameliorating a symptom of a hearing loss disorder in a subject, comprising providing to the subject a system, e.g., comprising at least one living electrode, comprising: (a) a biocompatible construct comprising a matrix comprising a first end, a second end, and a body; and (b) a plurality of auditory neurons (e.g., as described herein, e.g., vestibular neurons and/or cochlear neurons).

In some embodiments, a hearing loss disorder comprises one or more of conducting hearing loss, sensorineural hearing loss or mixed hearing loss. In some embodiments, a hearing loss disorder comprises one or more of acquired hearing loss disorder, sudden hearing loss disorder, secondary hearing loss (drug induced or injury induce hearing loss), autoimmune inner ear disease, otosclerosis, Meniere's disease, presbycusis, or acoustic neuroma.

In some embodiments of any of the methods disclosed herein, a subject is a human.

Also provided herein is a method of preventing the worsening of a symptom of a hearing loss disorder in a subject, comprising providing to the subject a system, e.g., comprising at least one living electrode, comprising: (a) a biocompatible construct comprising a matrix comprising a first end, a second end, and a body; and (b) a plurality of auditory neurons (e.g., as described herein, e.g., vestibular neurons and/or cochlear neurons).

In some embodiments, a hearing loss disorder comprises one or more of conducting hearing loss, sensorineural hearing loss or mixed hearing loss. In some embodiments, a hearing loss disorder comprises one or more of acquired hearing loss disorder, sudden hearing loss disorder, secondary hearing loss (drug induced or injury induce hearing loss), autoimmune inner ear disease, otosclerosis, Meniere's disease, presbycusis, or acoustic neuroma.

In some embodiments of any of the methods disclosed herein, a subject is a human.

This disclosure provides a kit comprising a system comprising: (a) a biocompatible construct comprising a first end, a second end, and a body; and (b) a plurality of auditory neurons, and instructions for using the same.

In some embodiments, a kit further comprises instructions for using the same.

In some embodiments, a kit described herein comprises a system described herein.

In some embodiments, any of the methods or kits disclosed herein comprises a system, e.g., comprising at least one living electrode, disclosed herein. In some embodiments, a system disclosed herein comprises: (a) a biocompatible construct comprising a matrix comprising a first end, a second end, and a body; and (b) a plurality of auditory neurons.

In some embodiments of any of the methods or kits disclosed herein, a biocompatible construct comprises an inner surface and/or an outer surface. In some embodiments, an inner surface of a biocompatible construct defines a luminal core.

In some embodiments of any of the methods or kits disclosed herein, an outer surface of a biocompatible construct comprises at least one hydrogel. In some embodiments, a hydrogel comprises a hydrophilic biopolymer and/or a synthetic polymer. In some embodiments, a hydrogel is at least partially cross-linked, wherein the cross-linking optionally increases stiffness, reduces porosity, and/or increases degradation time. In some embodiments, a hydrophilic biopolymer comprises one or more of agarose, hydrogel, hyaluronan, chitosan, alginate, collagen, dextran, pectin, carrageenan, polylysine, gelatin, hyaluronic acid, fibrin, and methylcellulose. In some embodiments, a hydrophilic biopolymer comprises agarose. In some embodiments, a hydrophilic biopolymer comprises hydrogel. In some embodiments, a hydrophilic biopolymer comprises chitosan. In some embodiments, a hydrophilic biopolymer comprises alginate. In some embodiments, a hydrophilic biopolymer comprises collagen. In some embodiments, a hydrophilic biopolymer comprises dextran. In some embodiments, a hydrophilic biopolymer comprises pectin. In some embodiments, a hydrophilic biopolymer comprises carrageenan. In some embodiments, a hydrophilic biopolymer comprises polylysine. In some embodiments, a hydrophilic biopolymer comprises gelatin. In some embodiments, a hydrophilic biopolymer comprises hyaluronic acid. In some embodiments, a hydrophilic biopolymer comprises fibrin. In some embodiments, a hydrophilic biopolymer comprises methylcellulose.

In some embodiments of any of the methods or kits disclosed herein, a hydrophilic biopolymer comprises agarose. In some embodiments, an agarose is at about 0.25-30%, about 0.25%-3%, about 0.5%-3%, about 1-20%, about 1.5-10%, about 2-9%, about 2.5-8%, or about 3-7%. In some embodiments, the agarose is at about 0.25-29%, about 0.25-28%, 0.25-%, about 0.25-27%, about 0.25-26%, about 0.25-25%, about 0.25-24%, about 0.25-23%, about 0.25-22%, about 0.25-21%, about 0.25-20%, about 0.25-19%, about 0.25-18%, about 0.25-17%, about 0.25-16%, about 0.25-15%, about 0.25-14%, about 0.25-13%, about 0.25-12%, about 0.25-11%, about 0.25-10%, about 0.25-9%, about 0.25-8%, about 0.25-7%, about 0.25-6%, about 0.25-5%, about 0.25-4%, about 0.25-3%, about 0.25-2%, about 0.25-1%, about 0.25-0.5%, about 0.5-30%, about 1-30%, about 2-30%, about 3-30%, about 4-30%, about 5-30%, about 6-30%, about 7-30%, about 8-30%, about 9-30%, about 10-30%, about 11-30%, about 12-30%, about 13-30%, about 14-30%, about 15-30%, about 16-30%, about 17-30%, about 18-30%, about 19-30%, about 20-30%, about 21-30%, about 22-30%, about 23-30%, about 24-30%, about 25-30%, about 26-30%, about 27-30%, about 28-30%, about 29-30%.

In some embodiments of any of the methods or kits disclosed herein, an agarose in a system disclosed herein is at about 0.25%, about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%. In some embodiments, an agarose is at about 3%.

In some embodiments of any of the methods or kits disclosed herein, a hydrophillic biopolymer comprises at least one synthetic hydrogel (e.g., Pluronic hydrogel) or at least one synthetic hydrogel made of amphiphilic copolymers consisting of units of ethylene oxide (PEO) and polypropylene oxide (PPO). In some embodiments, a hydrogel is at about 2-50% w/v, about 3-40% w/v, about 4-30% w/v, about 5-25% w/v or about 10-20% w/v. In some embodiments, a hydrogel is at about 2-45%, about 2-40%, about 2-35%, about 2-30%, about 2-29%, about 2-28%, about 2-27%, about 2-26%, about 2-25%, about 2-24%, about 2-23%, about 2-22%, about 2-21%, about 2-20%, about 2-19%, about 2-18%, about 2-17%, about 2-16%, about 2-15%, about 2-14%, about 2-13%, about 2-12%, about 2-11%, about 2-10%, about 2-9%, about 2-8%, about 2-7%, about 2-6%, about 2-5%, about 2-4%, about 2-3%, about 3-50%, about 4-50%, about 5-50%, about 6-50%, about 7-50%, about 8-50%, about 9-50%, about 10-50%, about 11-50%, about 12-50%, about 13-50%, about 14-50%, about 15-50%, about 16-50%, about 17-50%, about 18-50%, about 19-50%, about 20-50%, about 21-50%, about 22-50%, about 23-50%, about 24-50%, about 25-50%, about 26-50%, about 27-50%, about 28-50%, about 29-50%, about 30-50%, about 35-50%, about 40-50%, about 45-50% w/v. In some embodiments, a hydrogel is at about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 40%, about 45%, about 50% w/v. In some embodiments, a hydrogel is at about 28.1%, about 28.2%, about 28.3%, about 28.4%, about 28.5%, about 28.6%, about 28.7%, about 28.8%, about 28.9%, about 30%. In some embodiments, a hydrogel is at about 28.6% w/v.

In some embodiments of any of the methods or kits disclosed herein, an inner surface of a biocompatible construct comprises one or more extracellular matrix (ECM) components. In some embodiments, an ECM component comprises collagen, laminin, fibronectin, hyaluronic acid, or a combination thereof. In some embodiments, an ECM component comprises collagen. In some embodiments, an ECM component comprises laminin. In some embodiments, an ECM component comprises fibronectin. In some embodiments, an ECM component comprises hyaluronic acid.

In some embodiments, an ECM component comprises collagen. In some embodiments, a collagen is at a concentration of about 0.1-10 mg/ml. In some embodiments, a collagen is at a concentration of about 0.1-9 mg/ml, about 0.1-8 mg/ml, about 0.1-7 mg/ml, about 0.1-6 mg/ml, about 0.1-5 mg/ml, about 0.1-4 mg/ml, about 0.1-3 mg/ml, about 0.1-2 mg/ml, about 0.1-1 mg/ml, about 0.1-0.9 mg/ml, about 0.1-0.8 mg/ml, about 0.1-0.7 mg/ml, about 0.1-0.5 mg/ml, about 0.5-10 mg/ml, about 0.6-10 mg/ml, about 0.7-10 mg/ml, about 0.8-10 mg/ml, about 0.9-10 mg/ml, about 1-10 mg/ml, about 2-10 mg/ml, about 3-10 mg/ml, about 4-10 mg/ml, about 5-10 mg/ml, about 6-10 mg/ml, about 7-10 mg/ml, about 8-10 mg/ml, about 9-10 mg/ml. In some embodiments, a collagen is at a concentration of about 0.1 mg/ml, about 0.2 mg/ml, about 0.3 mg/ml, about 0.4 mg/ml, about 0.5 mg/ml, about 0.6 mg/ml, about 0.7 mg/ml, about 0.8 mg/ml, about 0.9 mg/ml, about 1 mg/ml, about 1.1 mg/ml, about 1.2 mg/ml, about 1.3 mg/ml, about 1.4 mg/ml, about 1.5 mg/ml, about 1.6 mg/ml, about 1.7 mg/ml, about 1.8 mg/ml, about 1.9 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml. In some embodiments, the collagen is at a concentration of about 1 mg/ml.

In some embodiments, an ECM component comprises laminin. In some embodiments, a laminin is at a concentration of about 0.1-9 mg/ml, about 0.1-8 mg/ml, about 0.1-7 mg/ml, about 0.1-6 mg/ml, about 0.1-5 mg/ml, about 0.1-4 mg/ml, about 0.1-3 mg/ml, about 0.1-2 mg/ml, about 0.1-1 mg/ml, about 0.1-0.9 mg/ml, about 0.1-0.8 mg/ml, about 0.1-0.7 mg/ml, about 0.1-0.5 mg/ml, about 0.5-10 mg/ml, about 0.6-10 mg/ml, about 0.7-10 mg/ml, about 0.8-10 mg/ml, about 0.9-10 mg/ml, about 1-10 mg/ml, about 2-10 mg/ml, about 3-10 mg/ml, about 4-10 mg/ml, about 5-10 mg/ml, about 6-10 mg/ml, about 7-10 mg/ml, about 8-10 mg/ml, about 9-10 mg/ml. In some embodiments, a laminin is at a concentration of about 0.1 mg/ml, about 0.2 mg/ml, about 0.3 mg/ml, about 0.4 mg/ml, about 0.5 mg/ml, about 0.6 mg/ml, about 0.7 mg/ml, about 0.8 mg/ml, about 0.9 mg/ml, about 1 mg/ml, about 1.1 mg/ml, about 1.2 mg/ml, about 1.3 mg/ml, about 1.4 mg/ml, about 1.5 mg/ml, about 1.6 mg/ml, about 1.7 mg/ml, about 1.8 mg/ml, about 1.9 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml. In some embodiments, a laminin is at a concentration of about 1 mg/ml.

In some embodiments of any of the methods or kits disclosed herein, a system, e.g., comprising at least one living electrode, comprises a plurality of auditory neurons comprising about 50-5000 auditory neurons, e.g., as described herein. In some embodiments, a plurality of auditory neurons comprises about 50-4500, about 50-4000, about 50-3500, about 50-3000, about 50-2500, about 50-2000, about 50-1500, about 50-1000, about 50-900, about 50-800, about 50-700, about 50-600, about 50-550, about 50-500, about 50-450, about 50-400, about 50-300, about 50-200, about 50-100, about 100-5000, about 200-5000, about 300-5000, about 400-5000, about 500-5000, about 1000-5000, about 2000-5000, about 3000-5000 or about 4000-5000 auditory neurons. In some embodiments, a plurality of auditory neurons comprises at least 50 auditory neurons, at least 100 auditory neurons, at least 200 auditory neurons, at least 300 auditory neurons, at least 400 auditory neurons, at least 450 auditory neurons, at least 500 auditory neurons, at least 550 auditory neurons, at least 600 auditory neurons, at least 700 auditory neurons, at least 800 auditory neurons, at least 900 auditory neurons, at least 1000 auditory neurons, at least 2000 auditory neurons, at least 3000 auditory neurons, at least 4000, or at least 5000 auditory neurons. In some embodiments, a plurality of auditory neurons comprises at least 500 auditory neurons.

In some embodiments of any of the methods or kits disclosed herein, at least one of a plurality of auditory neurons is in contact with a biocompatible construct.

In some embodiments of any of the methods or kits disclosed herein, at least one neuron in a plurality of neurons comprises a cell body and/or an axon. In some embodiments, at least one neuron in a plurality of neurons comprises a cell body. In some embodiments, at least one neuron in a plurality of neurons comprises an axon. In some embodiments, at least one neuron in a plurality of neurons comprises a cell body and an axon. In some embodiments, an axon is about 0.5-50 cm in length.

In some embodiments of any of the methods or kits disclosed herein, a portion of at least one neuron in the plurality of auditory neurons is positioned: (i) at a first end of the biocompatible construct; (ii) at a second end of the biocompatible construct; (iii) at, along or within a portion of a body of the biocompatible construct; and/or (iv) at, along, or within the entire body of the biocompatible construct. In some embodiments, a portion of at least one neuron in the plurality of auditory neurons is positioned at a first end of the biocompatible construct. In some embodiments, a portion of at least one neuron in the plurality of auditory neurons is positioned at a second end of the biocompatible construct. In some embodiments, a portion of at least one neuron in the plurality of auditory neurons is positioned at, along or within a portion of a body of the biocompatible construct. In some embodiments, a portion of at least one neuron in the plurality of auditory neurons is positioned at, along, or within the entire body of the biocompatible construct.

In some embodiments of any of the methods or kits disclosed herein, a portion of at least one neuron in the plurality of auditory neuron positioned at a first end or at a second end of the biocompatible construct comprises a cell body.

In some embodiments of any of the methods or kits disclosed herein, a portion of at least one neuron in the plurality of auditory neuron positioned at, along or within a portion of a body of the biocompatible construct comprises an axon. In some embodiments, a body of the biocompatible construct comprises an inner surface of the body. In some embodiments, an inner surface comprises a luminal core.

In some embodiments of any of the methods or kits disclosed herein, a portion of at least one neuron in the plurality of auditory neuron positioned at, along or within the entire body of the biocompatible construct comprises an axon. In some embodiments, the axon extends unidirectionally or bidirectionally. In some embodiments, the axon extends unidirectionally. In some embodiments, the axon extends bidirectionally.

In some embodiments of any of the methods or kits disclosed herein, at least a portion of the plurality of auditory neurons comprises an aggregate of auditory neurons.

In some embodiments of any of the methods or kits disclosed herein, a plurality of auditory neurons comprises a neuron having one or more characteristics of a neuron present in a cochlea, spiral ganglion, auditory nerve, auditory brainstem nuclei, and/or cortical brainstem nuclei of an organism, e.g., a human. In some embodiments, a plurality of auditory neurons comprises a neuron derived from a neuron present in a cochlea, spiral ganglion, auditory nerve, auditory brainstem nuclei, and/or cortical brainstem nuclei of an organism, e.g., a human. In some embodiments, a plurality of auditory neurons comprises a spiral ganglion neuron, an inferior colliculus (IC) auditory neuron, a cortical auditory neuron, a type I auditory neuron, or a type II auditory neuron. In some embodiments, a plurality of auditory neurons is or comprises a plurality of spiral ganglion neurons. In some embodiments, a plurality of auditory neurons is or comprises a plurality of IC auditory neurons. In some embodiments, a plurality of auditory neurons is or comprises a plurality of cortical auditory neurons. In some embodiments, a plurality of auditory neurons is or comprises a plurality of type I auditory neurons. In some embodiments, a plurality of auditory neurons is or comprises a plurality of type II auditory neurons.

In some embodiments of any of the methods or kits disclosed herein, a system, e.g., comprising at least one living electrode, further comprises one or more additional cells, such as astrocytes, oligodendrocytes, Schwann cells, microglia, or endothelial cells.

In some embodiments of any of the methods or kits disclosed herein, a plurality of auditory neurons is isolated from a subject.

In some embodiments of any of the methods or kits disclosed herein, a plurality of auditory neurons is or comprises at least one cell derived from a stem cell. In some embodiments, a stem cell comprises one or more of an induced pluripotent stem cell (iPSC), a fetal stem cell, or a tissue stem cell. In some embodiments, a stem cell comprises an iPSC. In some embodiments, a stem cell comprises a fetal stem cell. In some embodiments, a stem cell comprises a tissue stem cell. In some embodiments, an auditory neuron derived from a stem cell has one or more characteristics of a neuron present in a cochlea, spiral ganglion, auditory nerve, auditory brainstem nuclei, and/or cortical brainstem nuclei of an organism, e.g., a human.

In some embodiments of any of the methods or kits disclosed herein, a plurality of auditory neurons is autologous, allogeneic, or xenogeneic to a subject. In some embodiments, a plurality of auditory neurons is autologous to a subject. In some embodiments, a plurality of auditory neurons is allogeneic to a subject. In some embodiments, a plurality of auditory neurons is xenogeneic to a subject.

In some embodiments of any of the methods or kits disclosed herein, at least one neuron in the plurality of auditory neurons is genetically engineered or reprogrammed using a non-genetic technique. In some embodiments, at least one genetically engineered neuron comprises a sensor, an actuator, a receptor and/or a reporter.

In some embodiments of any of the methods or kits disclosed herein, at least one genetically engineered neuron in the plurality of auditory neurons comprises a reporter. In some embodiments, the reporter comprises a fluorescent reporter.

In some embodiments, at least one genetically engineered neuron in the plurality of auditory neurons comprises a receptor. In some embodiments, the receptor comprises one or more of a magnetoreceptor, an electromagnetic receptor, an electroreceptor, a hydroreceptor, a mechanoreceptor, an osmoreceptor, a thermoreceptor, and a piezoelectric ion channel.

In some embodiments, an electromagnetic receptor comprises one or more of an infrared receptors, a photoreceptor, or an ultraviolet receptors.

In some embodiments, a mechanoreceptor comprises one or both of a mechanosensory receptor, or a proprioreceptor.

In some embodiments, at least one genetically engineered neuron in the plurality of auditory neurons comprises a sensor. In some embodiments, the sensor comprises an optical sensor comprising one or more of a light-sensitive ion channel, a calcium sensor, and a membrane voltage sensor.

In some embodiments, a sensor comprises a calcium sensor comprising one or more of Aequorin, Cameleon, and GCaMP.

In some embodiments, a sensor comprises a chloride sensor comprising Clomeleon.

In some embodiments, a sensor comprises a membrane voltage sensor comprising Mermaid.

In some embodiments, at least one genetically engineered neuron in the plurality of auditory neurons comprises an actuator. In some embodiments, the actuator comprises an optical actuator comprising one or more of channelrhodopsin, halorhodopsin, and archaerhodopsin.

In some embodiments, at least one genetically engineered neuron in the plurality of auditory neurons comprising an optical sensor and/or an optical actuator is an optogenetic neuron.

In some embodiments of any of the methods or kits disclosed herein, a biocompatible construct is a tubular construct.

In some embodiments of any of the methods or kits disclosed herein, a biocompatible construct is a spiral or a wye. In some embodiments, a biocompatible construct has a curvature of about 240-900 degrees. In some embodiments, the curvature of the biocompatible construct is about 270 degrees.

In some embodiments, a system comprising a biocompatible construct has an outer diameter of about 0.1-40 mm. In some embodiments, a system has an outer diameter of about 0.1-35 mm, about 0.1-30 mm, about 0.1-25 mm, about 0.1-20 mm, about 0.1-15 mm, about 0.1-12 mm, about 0.1-11 mm, about 0.1-10 mm, about 0.1-9 mm, about 0.1-8 mm, about 0.1-7 mm, about 0.1-6.5 mm, about 0.1-6 mm, about 0.1-5 mm, about 0.1-4 mm, about 0.1-3 mm, about 0.1-2 mm, about 0.1-1 mm, about 0.1-0.5 mm, about 0.2-40 mm, about 0.3-40 mm, about 0.4-40 mm, about 0.5-40 mm, about 1-40 mm, about 2-40 mm, about 3-40 mm, about 4-40 mm, about 5-40 mm, about 6-40 mm, about 7-40 mm, about 8-40 mm, about 9-40 mm, about 10-40 mm, about 11-40 mm, about 12-40 mm, about 15-40 mm, about 20-40 mm, about 25-40 mm, about 30-40 mm, or about 35-40 mm. In some embodiments, a system has an outer diameter of about 0.1 mm, about 0.5 mm, about 1 mm, about 2 mm, about 3 mm, about 4 mm, about 5 mm, about 6 mm, about 6.5 mm, about 7 mm, about 8 mm, about 9 mm, about 10 mm, about 11 mm, about 12 mm, about 13 mm, about 14 mm, about 15 mm, about 20 mm, about 25 mm, about 30 mm, about 35 mm or about 40 mm. In some embodiments, a system has an outer diameter of about 1.5 mm.

In some embodiments of any of the methods or kits disclosed herein, a system comprising a biocompatible construct has a diameter of about 0.5-70 mm, about 0.5-60 mm, about 0.5-50 mm, about 0.4-50 mm, about 0.5-30 mm, about 0.5-20 mm, about 0.5-10 mm, about 1-70 mm, about 2-70 mm, about 3-70 mm, about 4-70 mm, about 5-70 mm, about 6-70 mm, about 7-70 mm, about 8-70 mm, about 9-70 mm about 10-70 mm, about 20-70 mm, about 30-70 mm about 40-70 mm about 50-70 mm. In some embodiments, the system has a diameter of about 0.1-10 mm, about 0.2-9 mm, about 0.3-8 mm, about 0.4-7 mm, about 0.5-6.5 mm. In some embodiments, a system has a diameter of about 0.5 mm, about 1 mm, about 2 mm, about 3 mm, about 4 mm, about 5 mm, about 6 mm, about 6.5 mm, about 7 mm, about 8 mm, about 9 mm, about 10 mm, about 20 mm, about 30 mm, about 40 mm, about 50 mm, about 60 mm, about 70 mm. In some embodiments, a system comprising a biocompatible construct has a diameter of about 7 mm.

In some embodiments, a system comprising a biocompatible construct has a pitch of about 0.1-10 mm, about 0.1-8 mm, about 0.1-6 mm, about 0.1-4 mm, about 0.1-3 mm, about 0.1-2 mm, about 0.1-1.5 mm, about 0.1-1 mm, about 0.5-10 mm, about 0.6-10 mm, about 0.7-10 mm, about 0.8-10 mm about 0.9-10 mm, about 1-10 mm, about 2-10 mm, about 4-10 mm, about 6-10 mm about 8-10 mm. In some embodiments, a system has a pitch of about 0.1 mm, about 0.2 mm, about 0.3 mm, about 0.4 mm, about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 3 mm, about 4 mm, about 5 mm, about 6 mm, about 7 mm, about 8 mm, about 9 mm, about 10 mm. In some embodiments, a system comprising a biocompatible construct has a pitch of about 1 mm.

In some embodiments a system used in any of the methods or kits disclosed herein, is manufactured, e.g., assembled, ex vivo or in vitro. In some embodiments, a system is manufactured, e.g., assembled, ex vivo. In some embodiments, a system is manufactured, e.g., assembled, in vitro.

In some embodiments, a system used in any of the methods or kits disclosed herein is capable of, or can be used to, modulate and/or assess at least one activity of at least one neuron in the plurality of neurons in the system and/or at least one activity of at least neuron in a subject. In some embodiments, a system is capable of, or can be used to, modulate at least one activity of at least one neuron in the plurality of neurons in the system. In some embodiments, a system is capable of, or can be used to, modulate at least one activity of at least one neuron in a subject. In some embodiments, a system is capable of, or can be used to, modulate at least one activity of at least one neuron in the plurality of neurons in the system and at least one neuron in a subject.

In some embodiments, modulating is or comprises unidirectional or bidirectional modulation. In some embodiments, modulating is or comprises unidirectional modulation. In some embodiments, modulating is or comprises bidirectional modulation.

In some embodiments, modulating comprises stimulating or inhibiting at least one neuron in the plurality of auditory neurons in the system and/or at least one neuron in a subject. In some embodiments, modulating comprises stimulating or inhibiting at least one neuron in the plurality of auditory neurons in the system. In some embodiments, modulating comprises stimulating or inhibiting at least one neuron in a subject. In some embodiments, modulating comprises stimulating or inhibiting at least one neuron in the plurality of auditory neurons in the system and at least one neuron in a subject.

In some embodiments, stimulating comprises electrical stimulation; optical stimulation; sound wave or vibration stimulation; magnetic stimulation; or a combination thereof. In some embodiments, stimulating comprises electrical stimulation. In some embodiments, stimulating comprises optical stimulation. In some embodiments, stimulating comprises sound wave or vibration stimulation. In some embodiments, stimulating comprises magnetic stimulation. In some embodiments, stimulating comprises electrical stimulation and optical stimulation.

In some embodiments, assessing comprises monitoring and/or recording. In some embodiments, assessing comprises monitoring. In some embodiments, assessing comprises recording. In some embodiments, assessing comprises monitoring and recording.

In some embodiments, at least one neuron in a subject is or comprises an auditory neuron. In some embodiments, an auditory neuron comprises a spiral ganglion neuron, an IC neuron, or a combination thereof.

In some embodiments, a system used in any of the methods or kits disclosed herein is capable of stimulating or inhibiting an auditory pathway in a subject. In some embodiments, a system is capable of stimulating an auditory pathway in a subject. In some embodiments, a system is capable of inhibiting an auditory pathway in a subject.

In some embodiments, a system used in any of the methods or kits disclosed herein is capable of stimulating or inhibiting a peripheral auditory pathway in a subject. In some embodiments, a system is capable of stimulating a peripheral auditory pathway in a subject. In some embodiments, a system is capable of inhibiting a peripheral auditory pathway in a subject.

In some embodiments, a system, e.g., comprising at least one living electrode, used in any of the methods or kits disclosed herein is implanted at, near, or within a cochlea in a subject; at, near, or within an inferior colliculus (IC) in a subject; at, near or within a cochlear nucleus in a subject; and/or at, near or within an auditory cortex in a subject. In some embodiments, a system is implanted at, near, or within a cochlea in a subject. In some embodiments, a system is implanted at a cochlea in a subject. In some embodiments, a system is implanted near a cochlea in a subject. In some embodiments, a system is implanted within a cochlea in a subject. In some embodiments, a system is implanted at, near, or within an inferior colliculus (IC) in a subject. In some embodiments, a system is implanted at an inferior colliculus (IC) in a subject. In some embodiments, a system is implanted near an inferior colliculus (IC) in a subject. In some embodiments, a system is implanted within an inferior colliculus (IC) in a subject. In some embodiments, a system is implanted at, near, or within a cochlear nucleus in a subject. In some embodiments, a system is implanted at a cochlear nucleus in a subject. In some embodiments, a system is implanted near a cochlear nucleus in a subject. In some embodiments, a system is implanted within a cochlear nucleus in a subject. In some embodiments, a system is implanted at, near, or within an auditory cortex in a subject. In some embodiments, a system is implanted at an auditory cortex in a subject. In some embodiments, a system is implanted near an auditory cortex in a subject. In some embodiments, a system is implanted within an auditory cortex in a subject.

In some embodiments, a plurality of systems disclosed herein, e.g., comprising at least one living electrode, used in any of the methods or kits disclosed herein is implanted at, near, or within a cochlea in a subject; at, near, or within an inferior colliculus (IC) in a subject; at, near or within a cochlear nucleus in a subject; and/or at, near or within an auditory cortex in a subject. In some embodiments, a first system disclosed herein and a second system disclosed herein are implanted in a subject. In some embodiments, a first system disclosed herein and a second system disclosed herein are the same, e.g., two similar systems disclosed herein are implanted in a subject. In some embodiments, a first system disclosed herein and a second system disclosed herein are different, e.g., two different systems disclosed herein are implanted in a subject. In some embodiments, a third or further system disclosed herein is implanted in a subject.

In some embodiments, a first system is implanted at, near, or within a cochlea in a subject. In some embodiments, a first system is implanted at a cochlea in a subject. In some embodiments, a system is implanted near a cochlea in a subject. In some embodiments, a first system is implanted within a cochlea in a subject. In some embodiments, a second system is implanted at, near, or within an inferior colliculus (IC) in a subject. In some embodiments, a second system is implanted at an inferior colliculus (IC) in a subject. In some embodiments, a second system is implanted near an inferior colliculus (IC) in a subject. In some embodiments, a system is implanted within an inferior colliculus (IC) in a subject.

In some embodiments, a first system is implanted at, near, or within a cochlea in a subject. In some embodiments, a first system is implanted at a cochlea in a subject. In some embodiments, a system is implanted near a cochlea in a subject. In some embodiments, a first system is implanted within a cochlea in a subject. In some embodiments, a second system is implanted at, near, or within a cochlear nucleus in a subject. In some embodiments, a second system is implanted at a cochlear nucleus in a subject. In some embodiments, a second system is implanted near a cochlear nucleus in a subject. In some embodiments, a system is implanted within a cochlear nucleus in a subject. In some embodiments, a second system is implanted at, near, or within an auditory cortex in a subject. In some embodiments, a second system is implanted at an auditory cortex in a subject. In some embodiments, a second system is implanted near an auditory cortex in a subject. In some embodiments, a system is implanted within an auditory cortex in a subject.

In some embodiments, an implanted system used in any of the methods or kits disclosed herein contacts at least one cell in a subject or at, near, or within areas of the auditory cortex in a subject. In some embodiments, at least one cell in a subject is an endogenous cell. In some embodiments, at least one cell comprises a neuron. In some embodiments, a neuron comprises an auditory neuron, e.g., as described herein. In some embodiments, an auditory neuron comprises one or more of a spiral ganglion neuron or an IC neuron.

In some embodiments, contacting of a system used in any of the methods or kits disclosed herein with at least one cell in a subject is determined to create a synapse between at least one neuron of the plurality of auditory neurons of the system and at least one cell in a subject. In some embodiments, a synapse is or comprises a neuronal synapse.

In some embodiments, an implanted system used in any of the methods or kits disclosed herein provides an accessible interface for modulating at least one neuron in the plurality of auditory neurons in the system, and/or at least one cell in a subject. In some embodiments, an implanted system provides an accessible interface for modulating at least one neuron in the plurality of auditory neurons in the system. In some embodiments, an implanted system provides an accessible interface for modulating at least one cell in the subject.

In some embodiments of any of the methods or kits disclosed herein, a biocompatible construct has a non-linear shape.

These, and other embodiments encompassed by the present disclosure, are described in more detail below and in the claims.

### BRIEF DESCRIPTION OF THE DRAWINGS

The following detailed description of exemplary embodiments of the invention will be better understood when read in conjunction with the appended drawings. For the purpose of illustrating the invention, non-limiting embodiments are shown in the drawings. It should be understood, however, that the invention is not limited to the precise arrangements and instrumentalities of the embodiments shown in the drawings.

FIG. 1 provides a schematic of a Micro-TENN Biohybrid Neural Interface. A hydrogel scaffold directs axonal growth from a population of neurons towards a targeted tissue of interest (in this case spiral ganglion cells in the cochlea, or native auditory neurons in the inferior colliculus) while the cell bodies remain accessible for electrical or optical stimulation.

FIGS. 2A-2F depict Simultaneous Optical Stimulation and Recording in Micro-TENNs. Cortical neuron micro-TENN transduced to express both an optical actuator and a fluorescent calcium reporter may be controlled and monitored with light. FIG. 2A: Phase image and confocal reconstruction at 10 DIV, with the left aggregate transduced with ChR2 and the right aggregate transduced with the calcium reporter RCaMP. FIG. 2B: The RCaMP+ aggregate from FIG. 2A under fluorescent microscopy during recording. FIG. 2C: Confocal reconstruction of FIG. 2B post-stimulation. ROIs containing single neurons were manually defined (white outlines). FIG. 2D: Normalized pixel intensity of ROIs within the RCaMP+ aggregate from FIGS. 2A-2C during stimulation. Grey lines indicate representative, user-defined ROIs, which were averaged to obtain a mean ROI of the aggregate (solid black line). The timestamps of a single train of 1 Hz, 100 ms stimulation pulses are shown as blue bands along the abscissa. The changes in pixel intensity due to stimulation of the input aggregate can be seen as sharp spikes occurring within the endogenous, large-amplitude slow-wave activity. FIG. 2E: Zoom-ins of the red insets from showing micro-TENN activity during (left) stimulation and after (right) optical stimulation. FIG. 2F: Average maximum ΔF/Fo across stimulation intensities. Statistical comparison revealed that stimulation with the control wavelength (620 nm) yielded significantly lower maximum ΔF/Fo than with 465 nm (*=p<0.05). Scale bars: 100 μm. These results validate optically-evoked responses in similar constructs as those featured in the current proposal.

FIG. 3 shows anatomically-inspired Micro-TENNs. Confocal reconstructions of a dopaminergic micro-TENN that emulates the nigrostriatal pathway in vitro: discrete neuronal population with long-projecting axon tracts.

FIG. 4 depicts composite immunohistochemical micrograph of SGN explant at 7 DIV. Antibodies for Hoechst (nuclei,), GFAP (astrocytes,), tuj-1 (axons/neuronal soma,), and synapsin (synapses,) are shown. Scale bar=100 μm.

FIGS. 5A-5D depict calcium imaging. FIG. 5A: Phase image of spiral ganglion aggregates (1, 2) plated at 18 DIV. FIG. 5B: Aggregates were transduced with GCaMP and live calcium imaging recordings revealed neuronal activity (B, B′, B″). (FIGS. 5C, 5D) ROIs containing single neurons were identified for both aggregates and mean pixel intensities were analyzed in MATLAB.

FIGS. 6A-6E show SGN LE using aggregated SGN within implantable microcolumns. FIG. 6A: Phase image of a spiral ganglion LE projecting thick axonal tracts at 14 DIV. FIG. 6B: The LE from FIG. 6A immunolabeled to stain cell nuclei (DAPI) (FIG. 6C) axons (beta tubulin-III) (FIG. 6D) and peripherin (FIG. 6E). Scale bar: 500 μm.

FIGS. 7A-7C depict cochlear construct design. FIG. 7A: Phase Contrast Micrograph of living electrode showing bend with off-center central microcolumn. A curvature in the microcolumn can be seen due to the off-center orientation of the axonal tract. This material is flexible and can deform to accommodate the curvature of the cochlea. The bottom panels show 270 deg 3D printed constructs at FIG. 7B: 50% and FIG. 7C: 75% completion to better accentuate the central channel (arrow).

FIG. 8 shows a novel neuronal “aggregate” method to create Micro-TENNs. a “forced cell aggregation” method was applied using gentle centrifugation within inverted pyramid micro-wells. These neuronal aggregates were then precisely seeded at the end of the hydrogel microcolumns. Over several DIV, robust unidirectional axonal extension was observed along the length of the microcolumn.

FIGS. 9A-9C depict a surgical approach to the cochlea in the right ear of an adult Sprague-Dawley rat. FIG. 9A: Auditory bulla has been opened to reveal the stapedial artery (striped arrow) running over the cochlear promontory and between the crura of the stapes. FIG. 9B: cochleostomy in the basal turn of the cochlea (white arrow). FIG. 9C: The otic capsule has been removed exposing the bony modiolus housing the spiral ganglion (black arrow)

FIGS. 10A-10D provide a cartoon of future construct design. FIG. 10A: Single optrode and neuronal body aggregate. FIG. 10B: Two-dimensional array of optrode and cell body pairs. This portion of the construct will remain in the auditory bulla or outside of brain-stem parenchyma. FIG. 10C: Cartoon of cochlear construct showing optrodes and neuronal aggregate located in auditory bulla. Black lines represent axonal cell tracts terminating at various positions along the cochlea.

FIG. 10D: Cartoon of inferior colliculus construct showing optrodes and neuronal aggregate located outside of the brainstem parenchyma. The lines represent axonal cell tracts terminating in various locations within the volume of the inferior colliculus.

### DEFINITIONS

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used herein, “synapse” refers to a junction between a neuron (e.g., an auditory neuron) and another cell, across which chemical communication flows.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

The term “biocompatible construct”, as used herein, refers to a construct comprising a matrix that does not cause significant harm to living tissue when placed in contact with such tissue, e.g., in vivo (e.g., over a period of time). In certain embodiments, a biocompatible construct is not toxic to cells (e.g., over a period of time). In certain embodiments, a biocompatible construct does not induce significant inflammation or other immune response, or other adverse effects when placed or implanted into a subject (e.g., over a period of time). In some embodiments, a matrix of a biocompatible construct comprises a scaffold. In some embodiments, a matrix of a biocompatible construct comprises an inner surface, an outer surface or both an inner surface and an outer surface. In some embodiments, an inner surface of a matrix of a biocompatible construct defines a luminal core. In some embodiments, an inner surface of a matrix of a biocompatible construct comprises a component disclosed herein. In some embodiments, an outer surface of a matrix of a biocompatible construct comprises a component disclosed herein. In some embodiments, a biocompatible construct comprises one or more additional components. In some embodiments, a biocompatible construct is a tubular construct, a curved construct, a wye shaped construct or a non-linear construct.

In general, the term “engineered” refers to the aspect of having been manipulated by the hand of man. For example, a cell or organism is considered to be “engineered” if it has been subjected to a manipulation, so that its genetic, epigenetic, and/or phenotypic identity is altered relative to an appropriate reference cell such as otherwise identical cell that has not been so manipulated In some embodiments, an engineered cell is one that has been manipulated so that it contains and/or expresses a particular agent of interest (e.g., a protein, a nucleic acid, and/or a particular form thereof) in an altered amount and/or according to altered timing relative to such an appropriate reference cell. As is common practice and is understood by those in the art, progeny of an engineered cell are typically still referred to as “engineered” even though the actual manipulation was performed on a prior entity.

As used herein, the term “living electrode” refers to a system comprising a biocompatible construct that includes a matrix; and a plurality of cells (e.g., auditory neurons). In some embodiments, a living electrode comprises one or more neurite extensions, e.g., axons and/or dendrites, that extend unidirectionally or bidirectionally. In some embodiments, a living electrode can be implanted into a subject. In some embodiments, a living electrode comprises an interface that is accessible for modulating an activity of at least one neuron in the plurality of neurons or an activity of at least one cell in a subject in which the living electrode is implanted.

As used herein, the term “subject” refers to an organism, typically a mammal (e.g., a human). In some embodiments, a subject is suffering from a hearing loss disorder. In some embodiments, a subject is susceptible to a hearing loss disorder. In some embodiments, a subject displays one or more symptoms or characteristics of a hearing loss disorder. In some embodiments, a subject does not display any symptom or characteristic of a hearing loss disorder. In some embodiments, a subject is someone with one or more features characteristic of susceptibility to or risk of a hearing loss disorder. In some embodiments, a subject is a patient. In some embodiments, a subject is an individual to whom diagnosis and/or therapy is and/or has been administered.

As used herein, the term “auditory neuron” refers to a neuron having one or more characteristics of a neuron present in a cochlea, spiral ganglion, auditory nerve, auditory brainstem nuclei, and/or cortical brainstem nuclei of an organism, e.g., a human. In some embodiments, a characteristic includes a physical characteristic and/or a cellular characteristic. In some embodiments, a physical characteristic comprises a size, shape, aggregation pattern, and/or motility. In some embodiments, a cellular characteristic comprises: an expression pattern of an entity (e.g., a lipid, a polypeptide, and/or a polynucleotide), a differentiation capacity, a proliferation capacity, a signaling pathway typically associated with an auditory neuron as described herein, and/or an ability to respond to stimuli, e.g., a physiologic or electrophysiologic response to stimulation. In some embodiments, a characteristic comprises an ability to form a synapse (e.g., with a cell, e.g., a neuron) and/or an ability to transduce a signal. In some embodiments, an auditory neuron is a neuron derived from or obtained from a neuron present in a cochlea, spiral ganglion, auditory nerve, auditory brainstem nuclei, and/or cortical brainstem nuclei of an organism, e.g., a human. In some embodiments, an auditory neuron is or comprises a neuron present in a cochlea. In some embodiments, an auditory neuron is or comprises a neuron present in a spiral ganglion. In some embodiments, an auditory neuron is or comprises a neuron present in an auditory nerve. In some embodiments, an auditory neuron is or comprises a neuron present in auditory brainstem nuclei. In some embodiments, an auditory neuron is or comprises a neuron present in cortical brainstem nuclei. In some embodiments, an auditory neuron is or comprises a spiral ganglion neuron, an inferior colliculus (IC) auditory neuron, a cortical auditory neuron, a type I auditory neuron, or a type II auditory neuron. In some embodiments, a plurality of auditory neurons comprises a spiral ganglion neuron, an inferior colliculus (IC) auditory neuron, a cortical auditory neuron, a type I auditory neuron, a type II auditory neuron, or a combination thereof.

### DETAILED DESCRIPTION

It is estimated that 15% of the world's population have some degree of hearing loss and that over 5% suffer from disabling deafness. When hearing loss has progressed to the point that amplification is no longer sufficient, cochlear implantation has become the standard of care. Cochlear implants (CI) stimulate the auditory nerve directly, bypassing damaged portions of the cochlea. The impact of cochlear implantation cannot be overstated and a majority of implantees obtain significant benefit from their devices. However, despite their success, cochlear implants do not restore natural hearing and even the most successful patients have difficulty understanding speech in noise, talking on the telephone and appreciating complex sounds such as music.

The fundamentals of CI electrode design have remained essentially unchanged since the first implants were developed in the 1960's—a linear array with a variable number of leads that is inserted along the length of the cochlea from its base. Advancements in electrode design have included multichannel arrays that exploit the tonotopic organization of the cochlea, precurved designs to mimic the shape of the cochlear duct and position leads closer to the spiral ganglion, and decreased stiffness to reduce damage on insertion and ensure placement in the appropriate scala. Despite these advancements, current spread and cross-stimulation limits the number of independent channels available. It is estimated that 30 to 50 independent channels are required to approximate normal hearing, while fewer than 10 are often achieved with current technology.

Several strategies have been developed to compensate for these limitations. Pre-operative imaging is being used to more precisely choose electrode sizes for individual patients, post-operative image-guided programming techniques can inform selective deactivation of interfering electrodes and provide initial frequency programming estimates, and intraoperative electrocochleography can monitor insertion trauma in real-time. Stimulation and encoding strategies have also been developed to more precisely guide current flow and stimulation sites within the cochlea and to mimic biological signal patterns.

For patients who cannot benefit from cochlear implantation, there is a single central implant approved in the United States. The Auditory Brainstem Implant (ABI) has a flat array of electrodes intended to be placed against the surface of the cochlear nucleus (CN). In contrast to the success of cochlear implants, just over 1000 ABI's have been placed worldwide and the performance has not matched that of cochlear implants in most cases. A modification of the ABI including both surface and penetrating electrodes attempted to exploit the tonotopic arrangement of the CN, however no improvement in outcomes was achieved. An alternative central implant, the auditory midbrain implant (AMI) with 20 ring electrodes along a single shank was intended to be implanted into the inferior colliculus (IC). While pre-clinical data was promising, in a small clinical trial outcome goals were not achieved, and the device remains investigational. Also disclosed herein are examples of emerging bioengineering efforts intended to overcome the obstacles detailed above and deliver the next generation of auditory implants.

### Alterations to the Cochlea

Several methods have been developed to alter the cochlear environment and improve the performance of existing electrode technology. Many of these are based on the induction of neuronal growth between the electrode and remaining spiral ganglion cells thus reducing the potential for current spread. In vitro studies have found several factors that can promote neurite extension in spiral ganglion cells including growth hormone, brain-derived neurotrophic factor, glial cell line-derived neurotrophic factor, ciliary neurotrophic factor, and erythropoietin. Alternatively, coatings of the electrode itself have been evaluated including oriented collagen and microtexturing to promote neurite outgrowth. Laminin coated electrodes, BDNF gene transfer, and chronic intrascalar growth factor were shown to induce neuronal outgrowth into the scala tympani potentially allowing for direct contact with implanted electrodes.

Techniques that attempt to preserve residual hearing following implantation and then provide both acoustic and electric hearing have shown superior outcomes as compared to electric-only stimulation. Because of this there has been great interest in methods to reduce the immediate and long-term inflammatory response in the cochlea to prevent delayed hearing loss. Coating electrodes with polymer or silicone fibers helped to prevent fibroblast growth, and there have been multiple techniques for the sustained release of anti-inflammatory agents within the cochlea following implantation.

### Regenerative Techniques

The damage to cochlear hair cells and spiral ganglion neurons that lead to sensorineural hearing loss are currently irreversible outside of specific rare conditions. The issues noted above with cochlear implants could be obviated by the ability to restore the natural function of the cochlea. Gene therapies have been proposed for the preservation or regeneration of hair cells and auditory neurons in the cochlea, and for treatment of specific genetic forms of hearing loss. These techniques require the ability to introduce therapeutic agents into the inner ear without causing additional damage and several nanotechnology techniques are being developed to address this constraint. Stem cell-based therapies, including induced pluripotent stem cells, have also shown promise for both hair cell and auditory nerve cell regeneration. Decellularized cochlea have been investigated to act as scaffolding to support and such regenerative strategies.

### Optical Stimulation

Optical stimulation has been proposed as an alternative to traditional electrical stimulation as it could avoid issues with current spread and has the frequency and intensity resolution required to simulate complex sounds. Virally-based optogenetic transformation of spiral ganglion cells and linear multichannel micro-LED arrays have been shown to be able to stimulate the auditory system in animal models. There are several important technical challenges to overcome before such technology can be applied in the clinical setting.

### Piezoelectric

Piezoelectric materials have the ability to convert between electrical and mechanical energy—a property that naturally lends itself to auditory applications. Piezoelectric materials have been investigated for auditory applications as thin films, electrospun fibers, and nanoparticles. Piezoelectric materials have also been proposed as key for the development of fully implantable hearing systems. Similar to optogenetic technologies, while the potential for piezoelectric materials to form the foundation of next generation auditory implants is great, there remain significant technological hurdles to overcome.

Therefore, there is an unmet need to develop auditory implants that can overcome the obstacles detailed above.

There have recently been significant advances in the development of transplantable micro-tissue engineered neural networks (micro-TENNs) for reconstruction of brain pathways and central nervous system regeneration. In some embodiments, a micro-TENN is also referred to as a system comprising a biocompatible construct and a plurality of auditory neurons, e.g., comprising at least one living electrode. In some embodiments, construct disclosed herein is generated in vitro by seeding populations of neurons within a biocompatible construct, e.g., a customizable soft hydrogel scaffolds, and the neuronal phenotypes are adaptable to the application of interest. In some embodiments, the neurons contained in these scaffolds extend neurites along pre-formed channels while the cell bodies remain in a defined location. In some embodiments, each transplanted neuronal cell axon within the micro-TENN synapses, e.g., directly onto the cell body of one or more neurons in the target tissue, thus eliminating the potential for current spread. In some embodiments, the construct can be placed so that the axons are directed to a location of interest while the cell bodies remain accessible. Any non-organic components of a neural interface, including traditional electrodes or optrodes used to stimulate the transplanted neuronal cells, remain external to the brain parenchyma or cochlea. This technique induces very little trauma at the time of implantation, and is less likely to cause chronic inflammation from biomechanical mismatch of traditional electrodes. In some embodiments, this configuration of the micro-TENN creates an accessible interface for stimulation or recording of neuronal tissue combined with synapse-specific stimulation where the axon interacts with native neurons—also referred to as a “living electrode” (LE) in some embodiments. In some embodiments, the neurons utilized in the construct can be optogenetically functionalized to take advantage of the benefits of optical stimulation outlined above.

The present disclosure is the first to provide a system (e.g., comprising at least one living electrode) comprising a biocompatible construct and a plurality of auditory neurons which can be implanted into a subject to, e.g., modulate an auditory neuron, and/or treat or prevent a hearing loss disorder. In some embodiments, application to the auditory system has several potential advantages over existing solutions. In some embodiments, a biocompatible construct comprising a matrix, e.g., scaffold, can be designed with the complex 3D anatomy of the IC or CN. Both the IC and CN have a basic tonotopic structure, but complex sounds such as speech may not obey this simplified model. In some embodiments, it is likely that to recreate these complex sounds, precise simulation throughout the volume of the nuclei will be required. In some embodiments, the biocompatibility of a system disclosed herein, e.g., living electrodes, may allow for a greater volume of auditory nuclei to be accessible while avoiding issues of chronic inflammation. In some embodiments, for cochlear applications, previous work has shown that axons can be grown to several centimeters in length to extend along the length of the cochlea and utilize established implantation techniques. In some embodiments, the cell bodies of the implanted neurons can remain in the mastoid where they are, e.g., stimulated either optically or electrically while their axons are positioned to terminate at variable depths along the length of the cochlea. In some embodiments, a system disclosed herein, e.g., living electrode constructs, can physiologically interact with spiral ganglion neuronal populations. In some embodiments, such interactions in vivo can benefit from the native SGN outgrowth strategies described above. In some embodiments, the specificity of stimulation has the potential, e.g., to exceed any options utilizing standard electrode technology.

Auditory implants are the only widely available method for restoration of a human sense. Significant advancements have been made in the decades since the first single-channel cochlear implant was shown to provide reliable auditory sensations and these have generally been inspired by the natural structure and function of the cochlea—gravitating towards more anatomically and physiologically friendly designs. However, to date these designs have remained constrained within the confines of traditional electrode technology. While the results have been encouraging for hundreds of thousands of patients suffering from hearing loss—a fundamental rethinking of the prosthetic interface will be required to advance the outcomes to those approaching natural hearing. As the methods for optimization of traditional electrode technology continue to advance, including electrode properties, drug elusion, surface coating, and cochlear adaptation; multiple additional lines of investigation including optogenetics, piezoelectric materials, nanotechnology, and living electrodes could hold promise to form the basis for entirely novel methods for the restoration of hearing. Accordingly, this disclosure provides, inter alia, a system (e.g., comprising at least one living electrode) comprising a biocompatible construct and a plurality of auditory neurons which can be implanted into a subject to, e.g., modulate an auditory neuron, and/or treat or prevent a hearing loss disorder.

### Systems Comprising a Biocompatible Construct and Auditory Neurons

Disclosed herein, inter alia, are systems (e.g., living electrodes) comprising: a biocompatible construct comprising a matrix; and a plurality of auditory neurons. Systems disclosed herein can be manufactured, e.g., assembled, in vitro or ex vivo. Systems disclosed herein can be implanted in a subject and can be used to modulate an auditory neuron in a subject. In some embodiments, a system disclosed herein is or comprises comprising at least one living electrode.

### Biocompatible Constructs

A biocompatible construct disclosed herein comprises a matrix, e.g., as disclosed herein. In some embodiments, the biocompatible construct disclosed herein comprises a first end, a second end, and a body. In some embodiments, the matrix of the biocompatible construct comprises an inner surface and/or an outer surface. In some embodiments, the inner surface of a biocompatible construct defines a luminal core.

In some embodiments, the matrix of the biocompatible construct comprises a scaffold. In some embodiments, the matrix of the biocompatible construct comprises a component described herein, e.g., a component of an inner surface as described herein, and/or a component of an outer surface as described herein. In some embodiments, the matrix of the biocompatible construct comprises one or more additional components, e.g., as disclosed herein.

As an example, an outer surface of the biocompatible construct disclosed herein comprises at least one hydrogel (e.g., as described herein). In some embodiments, the hydrogel comprises a hydrophilic biopolymer and/or a synthetic polymer. In some embodiments, a hydrogel is at least partially cross-linked, wherein the cross-linking optionally increases stiffness, reduces porosity, and/or increases degradation time. In some embodiments, the hydrophilic biopolymer comprises one or more of agarose, hydrogel, hyaluronan, chitosan, alginate, collagen, dextran, pectin, carrageenan, polylysine, gelatin, hyaluronic acid, fibrin, and methylcellulose. In some embodiments, the hydrophilic biopolymer comprises agarose. In some embodiments, the hydrophilic biopolymer comprises hydrogel. In some embodiments, the hydrophilic biopolymer comprises chitosan. In some embodiments, the hydrophilic biopolymer comprises alginate. In some embodiments, the hydrophilic biopolymer comprises collagen. In some embodiments, the hydrophilic biopolymer comprises dextran. In some embodiments, the hydrophilic biopolymer comprises pectin. In some embodiments, the hydrophilic biopolymer comprises carrageenan. In some embodiments, a hydrophilic biopolymer comprises polylysine. In some embodiments, the hydrophilic biopolymer comprises gelatin. In some embodiments, the hydrophilic biopolymer comprises hyaluronic acid. In some embodiments, the hydrophilic biopolymer comprises fibrin. In some embodiments, the hydrophilic biopolymer comprises methylcellulose.

In some embodiments, the hydrophilic biopolymer comprises agarose. In some embodiments, an agarose is at about 0.25-30%, about 0.25%-3%, about 0.5%-3%, about 1-20%, about 1.5-10%, about 2-9%, about 2.5-8%, or about 3-7%. In some embodiments, the agarose is at about 0.25-29%, about 0.25-28%, 0.25-%, about 0.25-27%, about 0.25-26%, about 0.25-25%, about 0.25-24%, about 0.25-23%, about 0.25-22%, about 0.25-21%, about 0.25-20%, about 0.25-19%, about 0.25-18%, about 0.25-17%, about 0.25-16%, about 0.25-15%, about 0.25-14%, about 0.25-13%, about 0.25-12%, about 0.25-11%, about 0.25-10%, about 0.25-9%, about 0.25-8%, about 0.25-7%, about 0.25-6%, about 0.25-5%, about 0.25-4%, about 0.25-3%, about 0.25-2%, about 0.25-1%, about 0.25-0.5%, about 0.5-30%, about 1-30%, about 2-30%, about 3-30%, about 4-30%, about 5-30%, about 6-30%, about 7-30%, about 8-30%, about 9-30%, about 10-30%, about 11-30%, about 12-30%, about 13-30%, about 14-30%, about 15-30%, about 16-30%, about 17-30%, about 18-30%, about 19-30%, about 20-30%, about 21-30%, about 22-30%, about 23-30%, about 24-30%, about 25-30%, about 26-30%, about 27-30%, about 28-30%, about 29-30%.

In some embodiments, an agarose is at about 0.25%, about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%. In some embodiments, an agarose is at about 3%.

In some embodiments, a hydrophilic biopolymer comprises at least one synthetic hydrogel (e.g., Pluronic hydrogel) or at least one synthetic hydrogel made of amphiphilic copolymers consisting of units of ethylene oxide (PEO) and polypropylene oxide (PPO). In some embodiments, the hydrogel is at about 2-50% w/v, about 3-40% w/v, about 4-30% w/v, about 5-25% w/v or about 10-20% w/v. In some embodiments, the hydrogel is at about 2-45%, about 2-40%, about 2-35%, about 2-30%, about 2-29%, about 2-28%, about 2-27%, about 2-26%, about 2-25%, about 2-24%, about 2-23%, about 2-22%, about 2-21%, about 2-20%, about 2-19%, about 2-18%, about 2-17%, about 2-16%, about 2-15%, about 2-14%, about 2-13%, about 2-12%, about 2-11%, about 2-10%, about 2-9%, about 2-8%, about 2-7%, about 2-6%, about 2-5%, about 2-4%, about 2-3%, about 3-50%, about 4-50%, about 5-50%, about 6-50%, about 7-50%, about 8-50%, about 9-50%, about 10-50%, about 11-50%, about 12-50%, about 13-50%, about 14-50%, about 15-50%, about 16-50%, about 17-50%, about 18-50%, about 19-50%, about 20-50%, about 21-50%, about 22-50%, about 23-50%, about 24-50%, about 25-50%, about 26-50%, about 27-50%, about 28-50%, about 29-50%, about 30-50%, about 35-50%, about 40-50%, about 45-50% w/v. In some embodiments, the hydrogel is at about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 40%, about 45%, about 50% w/v. In some embodiments, the hydrogel is at about 28.1%, about 28.2%, about 28.3%, about 28.4%, about 28.5%, about 28.6%, about 28.7%, about 28.8%, about 28.9%, about 30%. In some embodiments, the hydrogel is at about 28.6% w/v.

An inner surface of the biocompatible construct disclosed herein, in some embodiments, comprises one or more extracellular matrix (ECM) components. In some embodiments, the ECM component comprises collagen, laminin, fibronectin, hyaluronic acid, or a combination thereof. In some embodiments, the ECM component comprises collagen. In some embodiments, an ECM component comprises laminin. In some embodiments, the ECM component comprises fibronectin. In some embodiments, the ECM component comprises hyaluronic acid.

In some embodiments, the ECM component comprises collagen. In some embodiments, collagen is at a concentration of about 0.1-10 mg/ml. In some embodiments, collagen is at a concentration of about 0.1-9 mg/ml, about 0.1-8 mg/ml, about 0.1-7 mg/ml, about 0.1-6 mg/ml, about 0.1-5 mg/ml, about 0.1-4 mg/ml, about 0.1-3 mg/ml, about 0.1-2 mg/ml, about 0.1-1 mg/ml, about 0.1-0.9 mg/ml, about 0.1-0.8 mg/ml, about 0.1-0.7 mg/ml, about 0.1-0.5 mg/ml, about 0.5-10 mg/ml, about 0.6-10 mg/ml, about 0.7-10 mg/ml, about 0.8-10 mg/ml, about 0.9-10 mg/ml, about 1-10 mg/ml, about 2-10 mg/ml, about 3-10 mg/ml, about 4-10 mg/ml, about 5-10 mg/ml, about 6-10 mg/ml, about 7-10 mg/ml, about 8-10 mg/ml, about 9-10 mg/ml. In some embodiments, a collagen is at a concentration of about 0.1 mg/ml, about 0.2 mg/ml, about 0.3 mg/ml, about 0.4 mg/ml, about 0.5 mg/ml, about 0.6 mg/ml, about 0.7 mg/ml, about 0.8 mg/ml, about 0.9 mg/ml, about 1 mg/ml, about 1.1 mg/ml, about 1.2 mg/ml, about 1.3 mg/ml, about 1.4 mg/ml, about 1.5 mg/ml, about 1.6 mg/ml, about 1.7 mg/ml, about 1.8 mg/ml, about 1.9 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml. In some embodiments, collagen is at a concentration of about 1 mg/ml.

In some embodiments, the ECM component comprises laminin. In some embodiments, laminin is at a concentration of about 0.1-9 mg/ml, about 0.1-8 mg/ml, about 0.1-7 mg/ml, about 0.1-6 mg/ml, about 0.1-5 mg/ml, about 0.1-4 mg/ml, about 0.1-3 mg/ml, about 0.1-2 mg/ml, about 0.1-1 mg/ml, about 0.1-0.9 mg/ml, about 0.1-0.8 mg/ml, about 0.1-0.7 mg/ml, about 0.1-0.5 mg/ml, about 0.5-10 mg/ml, about 0.6-10 mg/ml, about 0.7-10 mg/ml, about 0.8-10 mg/ml, about 0.9-10 mg/ml, about 1-10 mg/ml, about 2-10 mg/ml, about 3-10 mg/ml, about 4-10 mg/ml, about 5-10 mg/ml, about 6-10 mg/ml, about 7-10 mg/ml, about 8-10 mg/ml, about 9-10 mg/ml. In some embodiments, laminin is at a concentration of about 0.1 mg/ml, about 0.2 mg/ml, about 0.3 mg/ml, about 0.4 mg/ml, about 0.5 mg/ml, about 0.6 mg/ml, about 0.7 mg/ml, about 0.8 mg/ml, about 0.9 mg/ml, about 1 mg/ml, about 1.1 mg/ml, about 1.2 mg/ml, about 1.3 mg/ml, about 1.4 mg/ml, about 1.5 mg/ml, about 1.6 mg/ml, about 1.7 mg/ml, about 1.8 mg/ml, about 1.9 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml. In some embodiments, laminin is at a concentration of about 1 mg/ml.

### Physical Characteristics of Biocompatible Constructs

A biocompatible construct disclosed herein can have one or more characteristics described herein and/or can be part of a system (e.g., living electrode) disclosed herein. In some embodiments, the biocompatible construct disclosed herein is a tubular construct. In some embodiments, the biocompatible construct disclosed herein is a spiral or a wye. In some embodiments, the biocompatible construct disclosed herein has a non-linear shape.

The biocompatible construct which is a spiral or a wye, can have a curvature of about 240-900 degrees. In some embodiments, the curvature of the biocompatible construct is about 240-850 degrees, about 240-800 degrees, about 240-750 degrees, about 240-700 degrees, about 240-650 degrees, about 240-600 degrees, about 240-550 degrees, about 240-500 degrees, about 240-450 degrees, about 240-400 degrees, about 240-350 degrees, about 240-300 degrees, about 240-290 degrees, about 240-280 degrees, about 240-270 degrees, about 240-260 degrees, about 240-250 degrees, about 250-900 degrees, about 260-900 degrees, about 270-900 degrees, about 280-900 degrees, about 290-900 degrees, about 300-900 degrees, about 350-900 degrees, about 400-900 degrees, about 450-900 degrees, about 500-900 degrees, about 550-900 degrees, about 600-900 degrees, about 650-900 degrees, about 700-900 degrees, about 750-900 degrees, about 800-900 degrees, about 850-900 degrees. In some embodiments, the curvature of the biocompatible construct is about 240 degrees, about 250 degrees, about 260 degrees, about 270 degrees, about 280 degrees, about 290 degrees, about 300 degrees, about 350 degrees, about 400 degrees, about 450 degrees, about 500 degrees, about 550 degrees, about 600 degrees, about 650 degrees, about 700 degrees, about 750 degrees, about 800 degrees, about 850 degrees, or about 900 degrees.

In some embodiments, the system comprising the biocompatible construct disclosed herein has an outer diameter of about 0.1-40 mm. In some embodiments, the system has an outer diameter of about 0.1-35 mm, about 0.1-30 mm, about 0.1-25 mm, about 0.1-20 mm, about 0.1-15 mm, about 0.1-12 mm, about 0.1-11 mm, about 0.1-10 mm, about 0.1-9 mm, about 0.1-8 mm, about 0.1-7 mm, about 0.1-6.5 mm, about 0.1-6 mm, about 0.1-5 mm, about 0.1-4 mm, about 0.1-3 mm, about 0.1-2 mm, about 0.1-1 mm, about 0.1-0.5 mm, about 0.2-40 mm, about 0.3-40 mm, about 0.4-40 mm, about 0.5-40 mm, about 1-40 mm, about 2-40 mm, about 3-40 mm, about 4-40 mm, about 5-40 mm, about 6-40 mm, about 7-40 mm, about 8-40 mm, about 9-40 mm, about 10-40 mm, about 11-40 mm, about 12-40 mm, about 15-40 mm, about 20-40 mm, about 25-40 mm, about 30-40 mm, or about 35-40 mm. In some embodiments, the system has an outer diameter of about 0.1 mm, about 0.5 mm, about 1 mm, about 2 mm, about 3 mm, about 4 mm, about 5 mm, about 6 mm, about 6.5 mm, about 7 mm, about 8 mm, about 9 mm, about 10 mm, about 11 mm, about 12 mm, about 13 mm, about 14 mm, about 15 mm, about 20 mm, about 25 mm, about 30 mm, about 35 mm or about 40 mm. In some embodiments, the system has an outer diameter of about 1.5 mm.

In some embodiments, the system comprising the biocompatible construct disclosed herein has a diameter of about 0.5-70 mm, about 0.5-60 mm, about 0.5-50 mm, about 0.4-50 mm, about 0.5-30 mm, about 0.5-20 mm, about 0.5-10 mm, about 1-70 mm, about 2-70 mm, about 3-70 mm, about 4-70 mm, about 5-70 mm, about 6-70 mm, about 7-70 mm, about 8-70 mm, about 9-70 mm about 10-70 mm, about 20-70 mm, about 30-70 mm about 40-70 mm about 50-70 mm. In some embodiments, the system has a diameter of about 0.1-10 mm, about 0.2-9 mm, about 0.3-8 mm, about 0.4-7 mm, about 0.5-6.5 mm. In some embodiments, the system has a diameter of about 0.5 mm, about 1 mm, about 2 mm, about 3 mm, about 4 mm, about 5 mm, about 6 mm, about 6.5 mm, about 7 mm, about 8 mm, about 9 mm, about 10 mm, about 20 mm, about 30 mm, about 40 mm, about 50 mm, about 60 mm, about 70 mm. In some embodiments, a system has a diameter of about 7 mm.

In some embodiments, the system comprising a biocompatible construct disclosed herein ha a pitch of about 0.1-10 mm, about 0.1-8 mm, about 0.1-6 mm, about 0.1-4 mm, about 0.1-3 mm, about 0.1-2 mm, about 0.1-1.5 mm, about 0.1-1 mm, about 0.5-10 mm, about 0.6-10 mm, about 0.7-10 mm, about 0.8-10 mm about 0.9-10 mm, about 1-10 mm, about 2-10 mm, about 4-10 mm, about 6-10 mm about 8-10 mm. In some embodiments, a system has a pitch of about 0.1 mm, about 0.2 mm, about 0.3 mm, about 0.4 mm, about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 3 mm, about 4 mm, about 5 mm, about 6 mm, about 7 mm, about 8 mm, about 9 mm, about 10 mm. In some embodiments, the system has a pitch of about 1 mm.

### Auditory Neurons

Auditory neurons transmit information from the cochlea to the cortex. Exemplary auditory neurons include spiral ganglion neurons, inferior colliculus (IC) auditory neurons, cortical auditory neurons, type I auditory neurons, or type II auditory neurons.

Systems disclosed herein for use in treating hearing loss disorders and/or restoring hearing in subjects with hearing loss, comprise a biocompatible construct (e.g., as described herein) and a plurality of auditory neurons. In some embodiments, the plurality of auditory neurons comprises a spiral ganglion neuron, an inferior colliculus (IC) auditory neuron, a cortical auditory neuron, a type I auditory neuron, or a type II auditory neuron. In some embodiments, the plurality of auditory neurons is or comprises a plurality of spiral ganglion neurons. In some embodiments, the plurality of auditory neurons is or comprises a plurality of IC auditory neurons. In some embodiments, the plurality of auditory neurons is or comprises a plurality of cortical auditory neurons. In some embodiments, the plurality of auditory neurons is or comprises a plurality of type I auditory neurons. In some embodiments, the plurality of auditory neurons is or comprises a plurality of type II auditory neurons.

In some embodiments, the plurality of auditory neurons comprises about 50-5000 auditory neurons, e.g., as described herein. In some embodiments, the plurality of auditory neurons comprises about 50-4500, about 50-4000, about 50-3500, about 50-3000, about 50-2500, about 50-2000, about 50-1500, about 50-1000, about 50-900, about 50-800, about 50-700, about 50-600, about 50-550, about 50-500, about 50-450, about 50-400, about 50-300, about 50-200, about 50-100, about 100-5000, about 200-5000, about 300-5000, about 400-5000, about 500-5000, about 1000-5000, about 2000-5000, about 3000-5000 or about 4000-5000 auditory neurons. In some embodiments, the plurality of auditory neurons comprises at least 50 auditory neurons, at least 100 auditory neurons, at least 200 auditory neurons, at least 300 auditory neurons, at least 400 auditory neurons, at least 450 auditory neurons, at least 500 auditory neurons, at least 550 auditory neurons, at least 600 auditory neurons, at least 700 auditory neurons, at least 800 auditory neurons, at least 900 auditory neurons, at least 1000 auditory neurons, at least 2000 auditory neurons, at least 3000 auditory neurons, at least 4000, or at least 5000 auditory neurons. In some embodiments, the plurality of auditory neurons comprises at least 500 auditory neurons.

In some embodiments, at least one of the plurality of auditory neurons is in contact with the biocompatible construct.

In some embodiments, at least one neuron in the plurality of neurons comprises a cell body and/or an axon. In some embodiments, at least one neuron in the plurality of neurons comprises a cell body. In some embodiments, at least one neuron in the plurality of neurons comprises an axon. In some embodiments, at least one neuron in the plurality of neurons comprises a cell body and an axon.

In some embodiments, an axon is about 0.5-50 cm, about 0.5-49 cm, about 0.5-48 cm, about 0.5-47 cm, about 0.5-46 cm, about 0.5-45 cm, about 0.5-44 cm, about 0.5-43 cm, about 0.5-42 cm, about 0.5-41 cm, about 0.5-40 cm, about 0.5-39 cm, about 0.5-38 cm, about 0.5-37 cm, about 0.5-36 cm, about 0.5-35 cm, about 0.5-34 cm, about 0.5-33 cm, about 0.5-32 cm, about 0.5-31 cm, about 0.5-30 cm, about 0.5-29 cm, about 0.5-28 cm, about 0.5-27 cm, about 0.5-26 cm, about 0.5-25 cm, about 0.5-24 cm, about 0.5-23 cm, about 0.5-22 cm, about 0.5-21 cm, about 0.5-20 cm, about 0.5-19 cm, about 0.5-18 cm, about 0.5-17 cm, about 0.5-16 cm, about 0.5-15 cm, about 0.5-14 cm, about 0.5-13 cm, about 0.5-12 cm, about 0.5-11 cm, about 0.5-10 cm, about 0.5-9 cm, about 0.5-8 cm, about 0.5-7 cm, about 0.5-6 cm, about 0.5-5 cm, about 0.5-4 cm, about 0.5-3 cm, about 0.5-2 cm, about 0.5-1, about 0.6-50 cm, about 0.7-50 cm, about 0.8-50 cm, about 0.9-50 cm, about 1-50 cm, about 2-50 cm, about 3-50 cm, about 4-50 cm, about 5-50 cm, about 6-50 cm, about 7-50 cm, about 8-50 cm, about 9-50 cm, about 10-50 cm, about 11-50 cm, about 12-50 cm, about 13-50 cm, about 14-50 cm, about 15-50 cm, about 16-50 cm, about 17-50 cm, about 18-50 cm, about 19-50 cm, about 20-50 cm, about 21-50 cm, about 22-50 cm, about 23-50 cm, about 24-50 cm, about 25-50 cm, about 26-50 cm, about 27-50 cm, about 28-50 cm, about 29-50 cm, about 30-50 cm, about 31-50 cm, about 32-50 cm, about 33-50 cm, about 34-50 cm, about 35-50 cm, about 36-50 cm, about 37-50 cm, about 38-50 cm, about 39-50 cm, about 40-50 cm, about 41-50 cm, about 42-50 cm, about 43-50 cm, about 44-50 cm, about 45-50 cm, about 46-50 cm, about 47-50 cm, about 48-50 cm, or about 49-50 cm in length.

In some embodiments, a portion of at least one neuron in the plurality of auditory neurons is positioned: (i) at a first end of the biocompatible construct; (ii) at a second end of the biocompatible construct; (iii) at, along or within a portion of a body of the biocompatible construct; and/or (iv) at, along, or within the entire body of the biocompatible construct. In some embodiments, a portion of the at least one neuron in the plurality of auditory neurons is positioned at a first end of the biocompatible construct. In some embodiments, a portion of the at least one neuron in the plurality of auditory neurons is positioned at a second end of the biocompatible construct. In some embodiments, a portion of the at least one neuron in the plurality of auditory neurons is positioned at, along or within a portion of a body of the biocompatible construct. In some embodiments, a portion of the at least one neuron in the plurality of auditory neurons is positioned at, along, or within the entire body of the biocompatible construct.

In some embodiments, a portion of the at least one neuron in the plurality of auditory neuron positioned at a first end or at a second end of the biocompatible construct comprises a cell body.

In some embodiments, a portion of the at least one neuron in the plurality of auditory neuron positioned at, along or within a portion of a body of the biocompatible construct comprises an axon. In some embodiments, the body of the biocompatible construct comprises an inner surface of the body. In some embodiments, the inner surface comprises a luminal core. In some embodiments, a portion of the at least one neuron in the plurality of auditory neuron positioned at, along or within the entire body of the biocompatible construct comprises an axon. In some embodiments, the axon extends unidirectionally or bidirectionally. In some embodiments, the axon extends unidirectionally. In some embodiments, the axon extends bidirectionally.

In some embodiments, at least a portion of the plurality of auditory neurons comprises an aggregate of auditory neurons.

In some embodiments, the plurality of auditory neurons is isolated from a subject.

In some embodiments, the plurality of auditory neurons is or comprises at least one cell derived from a stem cell. In some embodiments, a stem cell comprises one or more of an induced pluripotent stem cell (iPSC), a fetal stem cell, or a tissue stem cell. In some embodiments, the stem cell comprises an iPSC. In some embodiments, the stem cell comprises a fetal stem cell. In some embodiments, the stem cell comprises a tissue stem cell.

In some embodiments, the plurality of auditory neurons is autologous, allogeneic, or xenogeneic to a subject. In some embodiments, the plurality of auditory neurons is autologous to a subject. In some embodiments, the plurality of auditory neurons is allogeneic to a subject. In some embodiments, the plurality of auditory neurons is xenogeneic to the subject.

In some embodiments, at least one neuron in the plurality of auditory neurons is genetically engineered or reprogrammed using a non-genetic technique. In some embodiments, the at least one genetically engineered neuron comprises a sensor, an actuator, a receptor and/or a reporter.

In some embodiments, the at least one genetically engineered neuron in the plurality of auditory neurons comprises a reporter. In some embodiments, the reporter comprises a fluorescent reporter.

In some embodiments, the at least one genetically engineered neuron in the plurality of auditory neurons comprises a receptor. In some embodiments, the receptor comprises one or more of a magnetoreceptor, an electromagnetic receptor, an electroreceptor, a hydroreceptor, a mechanoreceptor, an osmoreceptor, a thermoreceptor, and a piezoelectric ion channel.

In some embodiments, the electromagnetic receptor comprises one or more of an infrared receptors, a photoreceptor, or an ultraviolet receptors.

In some embodiments, the mechanoreceptor comprises one or both of a mechanosensory receptor, or a proprioreceptor.

In some embodiments, the at least one genetically engineered neuron in the plurality of auditory neurons comprises a sensor. In some embodiments, the sensor comprises an optical sensor comprising one or more of a light-sensitive ion channel, a calcium sensor, and a membrane voltage sensor.

In some embodiments, the sensor comprises a calcium sensor comprising one or more of Aequorin, Cameleon, and GCaMP.

In some embodiments, the sensor comprises a chloride sensor comprising Clomeleon.

In some embodiments, the sensor comprises a membrane voltage sensor comprising Mermaid.

In some embodiments, the at least one genetically engineered neuron in the plurality of auditory neurons comprises an actuator. In some embodiments, the actuator comprises an optical actuator comprising one or more of channelrhodopsin, halorhodopsin, and archaerhodopsin.

In some embodiments, the at least one genetically engineered neuron in the plurality of auditory neurons comprising an optical sensor and/or an optical actuator is an optogenetic neuron.

### Methods of Using Systems Disclosed Herein

Systems comprising biocompatible constructs and a plurality of auditory neurons as disclosed herein can be used to modulate an auditory neuron in a subject, to treat a subject having a hearing loss disorder, to ameliorate a symptom of hearing loss in a subject, or to restore hearing in a subject. In some embodiments, the subject has been determined to have a hearing loss disorder. In some embodiments, the subject has a symptom characteristic of a hearing loss disorder. In some embodiments, the subject is at risk of developing a hearing loss disorder. In some embodiments, the subject has a hearing loss disorder.

In some embodiments, the hearing loss disorder comprises one or more of conducting hearing loss, sensorineural hearing loss or mixed hearing loss. In some embodiments, the hearing loss disorder comprises one or more of acquired hearing loss disorder, sudden hearing loss disorder, secondary hearing loss (drug induced or injury induce hearing loss), autoimmune inner ear disease, otosclerosis, Meniere's disease, presbycusis, or acoustic neuroma.

In some embodiments, the subject is a human.

In some embodiments, the method described herein further comprises forming an aggregate of at least a portion of the plurality of auditory neurons. In some embodiments, the aggregate of at least a portion of the plurality of auditory neurons is formed prior to contacting the plurality of auditory neurons with a biocompatible construct.

In some embodiments of any of the methods disclosed herein, the method comprises implanting the system disclosed herein in the subject. In some embodiments, the system is implanted at, near, or within a cochlea in the subject, or at, near, or within an inferior colliculus (IC) in a subject. In some embodiments, the system is implanted at, near, or within a cochlea in the subject. In some embodiments, the system is implanted at, near, or within an inferior colliculus (IC) in the subject. In some embodiments, an implanted system contacts at least one cell in the subject or at, near, or within areas of the auditory cortex in the subject. In some embodiments, at least one cell in the subject is an endogenous cell. In some embodiments, at least one cell comprises an auditory neuron, e.g., as described herein. In some embodiments, an auditory neuron comprises one or more of a spiral ganglion neuron or an IC neuron.

In some embodiments, contacting of the system with at least one cell in the subject is determined to create a synapse between at least one neuron of the plurality of auditory neurons of the system and at least one cell in a subject. In some embodiments, the synapse is or comprises a neuronal synapse.

In some embodiments, an implanted system provides an accessible interface for modulating at least one activity of at least one neuron in the plurality of auditory neurons in the system, and/or at least one activity of at least one cell in the subject. In some embodiments, the implanted system provides an accessible interface for modulating at least one activity of at least one neuron in the plurality of auditory neurons in the system. In some embodiments, the implanted system provides an accessible interface for modulating at least one activity of at least one cell in the subject. In some embodiments, the implanted system provides an accessible interface for modulating at least one activity of at least one neuron in the plurality of auditory neurons in the system, and at least one activity of at least one cell in the subject.

Methods of using the system disclosed herein comprise modulating and/or assessing at least one activity of at least one neuron. In some embodiments, the system is capable of, or can be used to, modulate at least one activity of at least one neuron in the plurality of neurons in the system and/or at least one activity of at least one neuron in a subject. In some embodiments, the system is capable of, or can be used to, modulate at least one activity of at least one neuron in the plurality of neurons in the system. In some embodiments, the system is capable of, or can be used to, modulate at least one activity of at least one neuron in the subject. In some embodiments, a system is capable of, or can be used to, modulate at least one activity of at least one neuron in the plurality of neurons in the system and at least one activity of at least one neuron in the subject.

In some embodiments, modulating is or comprises unidirectional or bidirectional modulation. In some embodiments, modulating is or comprises unidirectional modulation. In some embodiments, modulating is or comprises bidirectional modulation.

In some embodiments, modulating comprises stimulating or inhibiting at least one neuron in the plurality of auditory neurons in the system and/or at least one neuron in the subject. In some embodiments, modulating comprises stimulating or inhibiting at least one neuron in the plurality of auditory neurons in the system. In some embodiments, modulating comprises stimulating or inhibiting at least one neuron in the subject. In some embodiments, modulating comprises stimulating or inhibiting at least one neuron in the plurality of auditory neurons in the system and at least one neuron in the subject.

In some embodiments, stimulating comprises electrical stimulation; optical stimulation; sound wave or vibration stimulation; magnetic stimulation; or a combination thereof. In some embodiments, stimulating comprises electrical stimulation. In some embodiments, stimulating comprises optical stimulation. In some embodiments, stimulating comprises sound wave or vibration stimulation. In some embodiments, stimulating comprises magnetic stimulation. In some embodiments, stimulating comprises electrical stimulation and optical stimulation.

In some embodiments, assessing comprises monitoring and/or recording. In some embodiments, assessing comprises monitoring. In some embodiments, assessing comprises recording. In some embodiments, assessing comprises monitoring and recording.

In some embodiments, at least one neuron in the subject is or comprises an auditory neuron. In some embodiments, the auditory neuron comprises a spiral ganglion neuron, an IC neuron, or a combination thereof.

In some embodiments of any of the methods disclosed herein, the system is capable of stimulating or inhibiting an auditory pathway in the subject. In some embodiments, the system is capable of stimulating an auditory pathway in the subject. In some embodiments, the system is capable of inhibiting an auditory pathway in the subject.

In some embodiments of any of the methods disclosed herein, the system is capable of stimulating or inhibiting a peripheral auditory pathway in the subject. In some embodiments, the system is capable of stimulating a peripheral auditory pathway in the subject. In some embodiments, the system is capable of inhibiting a peripheral auditory pathway in the subject.

### EXAMPLES

The invention is further described in detail by reference to the following experimental examples. These examples are provided for purposes of illustration only, and are not intended to be limiting unless so specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: Research Plan

### Background and Significance

It is estimated that 15% of the world's population have some degree of hearing loss and that over 5% suffer from disabling deafness. Further, there is increasing evidence that hearing loss leads to social isolation and has been correlated with dementia and cognitive decline. The most common causes of adult hearing loss include aging, noise exposure, ototoxicity, and diseases of the inner ear; and acquired hearing loss is often associated with deterioration and loss of cochlear hair cells. Disability from hearing loss is especially prevalent in the Veteran population due to significant noise exposure which has resulted in hearing loss and tinnitus being the most frequent service-connected disabilities. Often, mild to moderate hearing loss can be adequately rehabilitated with conventional hearing aids. However, when the hearing loss becomes severe, or the ability to discriminate speech becomes impaired, hearing aids are no longer sufficient. Cochlear implantation is the only technology that can restore a human sense and has become the standard of care for patients with profound deafness. Worldwide, over 200,000 patients have been implanted, with over 60,000 in the United States alone. There is also evidence that cochlear implants can provide tinnitus suppression while activated. Cochlear implantation was recently approved by the FDA for patients with single sided deafness, and one of the primary benefits in this population is tinnitus suppression. Though encouraging for many with severe hearing loss, there is a subset of patients who cannot benefit from cochlear implants due damage of either the cochlear nerve or the cochlea itself. The only option for any auditory perception for these patients is direct stimulation of the central auditory pathways through an auditory brainstem implant.

Cochlear implants: Cochlear implants use electrical impulses to stimulate the auditory nerve directly, bypassing damaged portions of the cochlea. Advances in surgical technique and coding strategies have allowed a majority of implantees to obtain significant benefit from their devices. These improvements have been so profound that many of the metrics used to measure outcomes fail to capture a complete picture of a patient's performance and there is increasing emphasis on quality of life outcomes to better define the impact an implant can have. Most cochlear implant users report a significant improvement in quality of life, and specific sub-populations including children, and the elderly show distinct improvements as well. Interestingly, quality of life metrics have been found to be only loosely correlated with traditional outcome measures such as speech understanding scores, which indicates impact well beyond traditional measures of success. However, despite their success, cochlear implants do not restore natural hearing. Even the most successful patients have difficulty understanding speech in noise, talking on the telephone, and appreciating complex sounds such as music. Though there has been evolution in electrode design, the basic principles have remained fundamentally unchanged since the first implants were developed in the 1960's—a linear electrode array with a variable number of leads that is inserted along the length of the cochlea from its base at or near the round window.

Currently available standard electrode designs in the U.S. vary between 12 (MED-EL), 16 (Advanced Bionics) and 22 (Cochlear Ltd) leads. In theory, a greater number of channels should lead to greater stimulation specificity and improved performance. In practice, current spread and cross-stimulation between electrodes limits the benefits of additional channels. All modern designs leave space between the electrode and the auditory nerve. This allows for current to spread to the surrounding electrolyte-rich fluid; thus, reducing the number of electrodes that can be independently active at a given time. This problem combined with inconsistencies in electrode placement have necessitated the development of image-guided programming techniques where electrodes likely to interfere with one another are selectively deactivated thus reducing cross-talk, but also limiting the number of channels available to transmit auditory information. It is estimated that approximately 30 to 50 independent channels are required to approximate normal hearing, while fewer than 10 are often achieved with current technology. Increasing the number of independent channels available for stimulation is needed for more challenging listening situations including understanding speech in noise and appreciating music. Attempts have been made to reduce the distance between the leads and spiral ganglion cells by pre-curving electrodes, however there has not been a significant increase in clinical performance and pre-curved electrodes tend to cause more damage to the delicate structures of the cochlea upon insertion than do lateral wall electrodes. Another method that has been tried to reduce the potential for current spread is to induce neuronal growth between the electrode and remaining spiral ganglion cells. In vitro studies have found several factors that can promote neurite extension in cultured spiral ganglion cells including growth hormone, brain-derived neurotrophic factor, glial cell line-derived neurotrophic factor, ciliary neurotrophic factor, and erythropoietin. Additionally, coatings of the electrode itself have been evaluated including polymer or silicone fibers to prevent fibroblast growth, and oriented collagen (38) and microtexturing to promote neurite outgrowth, amongst many others. Specifically, laminin coated electrodes were found to promote ‘extension of neurites through the osseus spiral lamina into the scala tympani’, BDNF gene transfer could induce ‘regenerated [SGN] fibres throughout the fibre tracks in the osseous spiral lamina and ectopic branching into both scala tympani and scala media’, and chronic intrascalar growth factor ‘induced fibers [to] even penetrate through canaliculi perforantes at the modiolar wall into [the] scala tympani’. Optical stimulation has also been proposed as an alternative to avoid current spread and has been shown to have improved temporal and dynamic rage properties for auditory stimulation, however the induction of light-sensitivity to native cells in the cochlea and introduction of a light source have proved significant technical challenges. Though promising in the lab, none of these methods have approached clinical applicability. It seems a limit has been reached in the fidelity of hearing achievable through current cochlear implant electrode design and a fundamental advance in electrode technology is required to provide an auditory experience that can approximate natural hearing.

Central implants: For patients who cannot benefit from cochlear implantation, there is a single central implant currently approved in the United States. This device is FDA approved for patients with neurofibromatosis type II (NFII) who are over the age of twelve years however any patient who cannot receive a cochlear implant due to anatomical reasons could potentially benefit—those who have had their cochlear nerve cut or damaged due to surgery or trauma, who have an inaccessible cochlea due to scarring or ossification, or have had their cochlea removed for surgical access to a skull base tumor.

The Auditory Brainstem Implant (ABI) is based on a cochlear implant platform, however in place of a linear array meant to be inserted into the cochlea, there is a flat array of electrodes that is placed against the surface of the brainstem intended to stimulate second order neurons of the cochlear nucleus (CN). In contrast to cochlear implants which have been implanted in hundreds of thousands of patients, just over 1000 ABI's have been placed worldwide. There have been a few cases of patients achieving open set speech (understanding speech with no visual ques), however the majority of patients achieve little more than sound awareness. Most patients receiving these devices have had large vestibular schwannomas that distort the normal brainstem anatomy which may affect the accuracy of placement. Patients receiving ABI for other indications have somewhat better results, but in most cases could not approach cochlear implant performance.

An alternative central implant was created to sidestep some of the anatomical issues with cochlear nucleus insertion and introduce a novel electrode design. The auditory midbrain implant (AMI) has an electrode that is a single shank with 20 ring electrodes along its length intended to be implanted into the inferior colliculus (IC). While pre-clinical data was promising, a clinical trial involving five NF-2 patients found that although some sound awareness was realized, none were able to achieve open set speech recognition. This device is still investigational and is not available clinically.

Criticism of both electrode array types revolves around the inability to specifically interact with the complex 3D anatomy of the CN or IC. Even the penetrating shank electrode of the AMI can only provide distinct stimulation at different depths along a single axis, but not along its length or width. Both the IC and CN have a basic tonotopic structure, but complex sounds such as speech may not obey this simplified model. It is likely that to recreate these complex sounds, precise simulation throughout the volume of the nuclei will be required. An alternative electrode design with both surface and penetrating electrodes (referred to as the penetrating ABI or PABI) attempted to overcome this limitation. The penetrating electrodes were found to require lower charge levels to achieve auditory sensations than the surface electrodes, however few provided any auditory sensation at all and there was actually slightly poorer performance in the PABI patients as compared to those receiving the standard ABI surface electrode. Thus, no central auditory implant has been able to approach the outcomes of cochlear implants, and often only sound awareness can be achieved.

A theoretical advantage of central implants over cochlear implants is that the delicate 3D anatomy of the cochlea is easily damaged upon electrode insertion and is extremely susceptible to chronic inflammatory reactions common with long-term indwelling foreign bodies—both of which cause loss of residual hearing. Bypassing the cochlea and placing central implants would avoid these issues and could potentially allow for implantation to augment natural hearing earlier in the course of decline. This advantage is offset by the increased morbidity of access to the brainstem. With the advance of minimally invasive skull base procedures, this limitation may be overcome, and central implantation could become feasible for patients not receiving adequate benefit from their hearing aids, with comparable operative risk as current cochlear implants.

Regenerative techniques for hearing restoration: Sound energy entering the ear is transduced to electrical signals in the spiral ganglion neurons via the inner hair cells (IHC) located in the Organ of Corti. Acquired hearing loss is most commonly secondary to deterioration and eventual loss of these cells and in humans these cells have no capacity for regeneration. There are rare and specific forms of hearing loss that may respond to medical interventions. Sudden hearing loss is sensorineural hearing loss developing over hours to days and is thought to be due to viral infiltration of the inner ear and subsequent inflammatory response. If caught early, there is some evidence that steroids can improve the chances of recovery. Similarly, immunosuppression can restore hearing, or prevent further loss in patients with autoimmune inner ear disease. This is an extraordinarily rare disorder characterized by progressive hearing loss in both ears that is responsive to steroid treatment. It may occur alone, or as part of a more systemic hearing loss. Both of these conditions are uncommon and in general, sensorineural hearing loss is considered irreversible with treatments available in the clinic today.

Gene therapies for the restoration of hearing have been proposed with several potential goals: preservation or regeneration of hair cells and auditory neurons in the cochlea, or for treatment of genetic forms of hearing loss. These techniques require the ability to introduce therapeutic agents into the inner ear without causing additional damage and several novel nanotechnology techniques are being developed to address this constraint. Stem cell-based therapies, including induced pluripotent stem cells have shown promise for both hair cell and auditory nerve cell regeneration. Although there is currently one ongoing U.S. clinical gene therapy trial utilizing CGF166, a recombinant adenovirus 5 (Ad5) vector containing the human Atonal transcription factor (Hath1) cDNA, no gene or cell-based therapy has been approved for clinical use at this time.

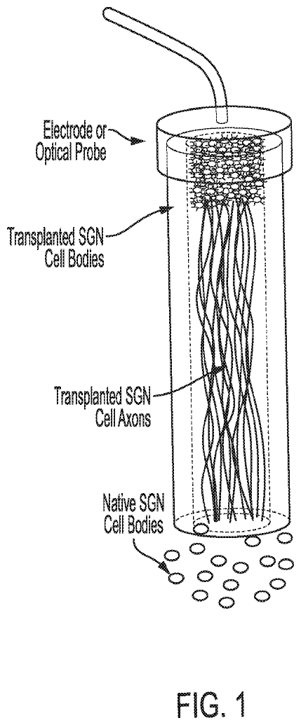

Living electrode technology: The inventors of the present disclosure have pioneered techniques to create anatomically-inspired micro-tissue engineered neural networks (micro-TENNs) for reconstruction of brain pathways (Struzyna, L. A. et al. Tissue Eng Pt A 21, 2744-2756 (2015), Cullen, D. K. et al. Tissue Eng Pt A 18, 2280-2289 (2012), Struzyna, L. A. et al. J Vis Exp Jove e55609 (2017) doi:10.3791/55609, Harris, J. P. et al. J Neural Eng 13, 016019 (2016)). These are transplantable tissue engineered “living scaffolds” that can facilitate central nervous system regeneration). The constructs are generated in vitro by seeding discreet populations of neurons within soft hydrogel scaffolds. The neuronal phenotypes and configuration of the hydrogel scaffold are customized to the application of interest. The neurons contained in these scaffolds extend neurites along pre-formed channels in the scaffold while the cell bodies remain in a defined location. Traditional electrodes induce a current that results in target-tissue depolarization of all cell bodies within a volume surrounding the electrode lead, while each transplanted neuronal cell axon within the micro-TENN synapses directly onto the cell body of one or more neurons in the target tissue, thus eliminating the potential for current spread. The construct is placed so that the axons are directed to a location of interest within the brain parenchyma while the cell bodies remain accessible at the surface (FIG. 1). Any non-organic components of a neural interface, including traditional electrodes or optrodes used to stimulate the transplanted neuronal cells, remain external to the brain parenchyma. In some embodiments, this technique induces very little trauma at the time of implantation (58), and is less likely to cause chronic inflammation from biomechanical mismatch of traditional electrodes.

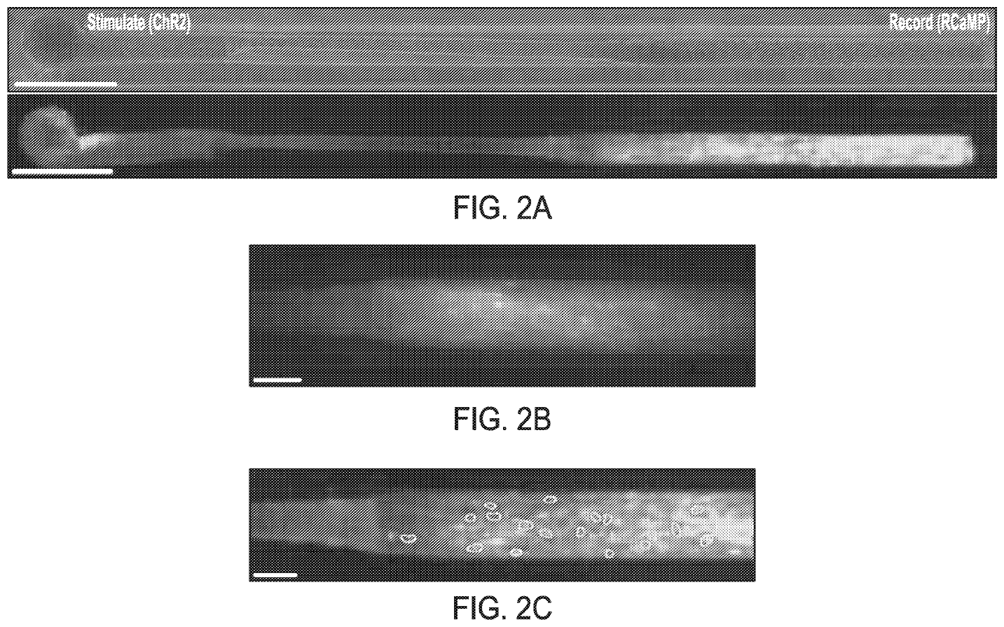

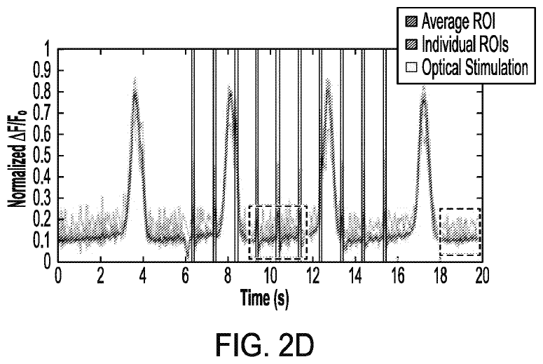

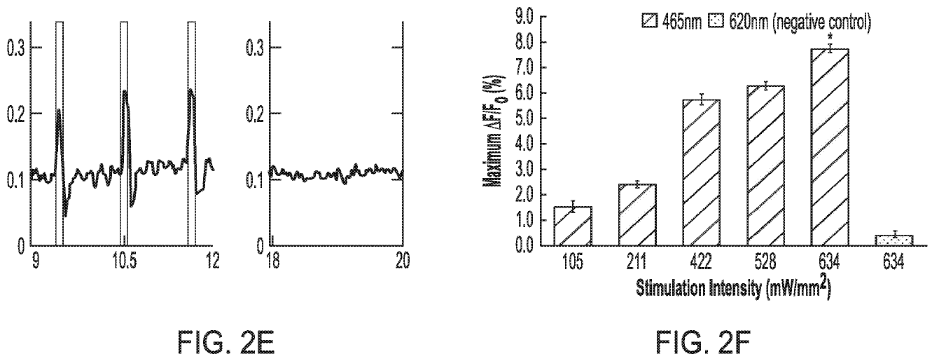

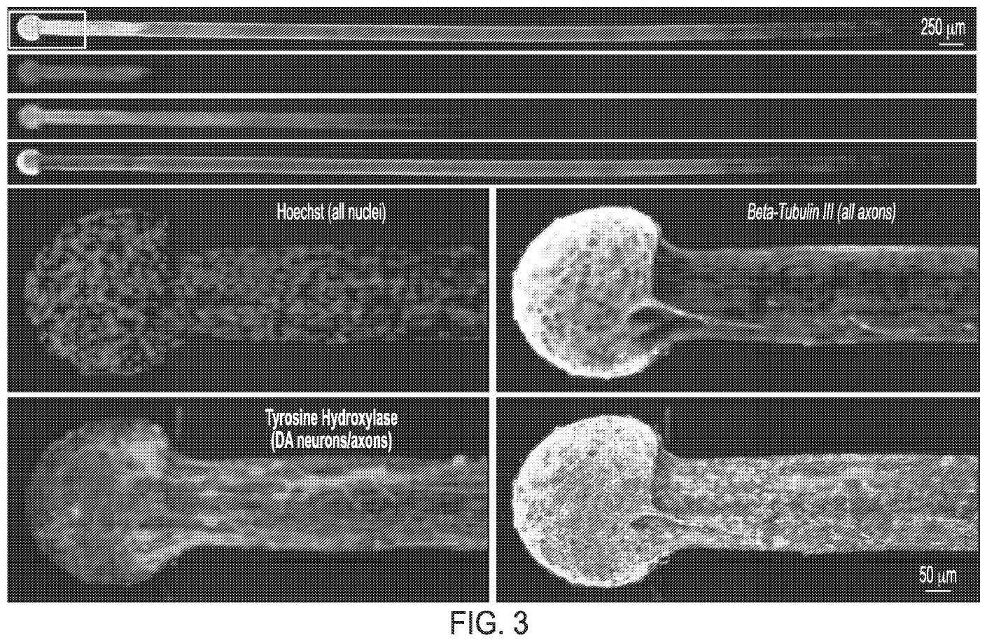

In some embodiments, this configuration of the micro-TENN creates an accessible interface for stimulation or recording of neuronal tissue combined with synapse-specific stimulation where the axon interacts with native neurons—essentially a “living electrode” (LE), also referred to herein, in some embodiments, as a system comprising a biocompatible construct and a plurality of auditory neurons. In some embodiments, the constructs are further functionalized via transduction with channelrhodopsins to enable light-based activation or monitoring of the accessible neuronal cell bodies (FIGS. 2A-2F). FIG. 3 shows an example of a micro-TENN generated—in this case built to restore dopaminergic axonal inputs in patients afflicted by Parkinson's disease. Multiple neuronal subtypes have been successfully used to generate micro-TENNs including primary cerebral cortical (glutamatergic) neurons, dorsal root ganglion neurons, GABAergic neurons, and dopaminergic neurons.

Importantly, application of this technology to the auditory system has not yet been explored, and it holds particular advantages over the current central and cochlear implants in several important ways. In some embodiments, the most direct translation from the existing work is to central implants. In some embodiments, the hydrogel scaffold can be designed with the complex 3D anatomy of the IC or CN in mind. For example, groups of constructs as described above can be combined in custom three-dimensional configurations to create a multi-living electrode array. In some embodiments, axons can not only be directed to synapse at precise locations along the length and width of the nucleus, but also at precise depths to reach the tonotopic configuration of either nuclei while the transplanted neuronal cell bodies remain accessible on the brainstem surface or even on the surface of the skull. Previous work has shown that axons can be grown to several centimeters in length. This length can be exploited to spatially separate individual aggregates of neuronal cells bodies on the brain surface while precisely targeting the axonal projections to conform to the more compact three-dimensional arrangement of the volume of the IC or CN. In some embodiments, the specificity of stimulation has the potential to exceed any options utilizing standard electrode technology. In fact, it has been shown that optical and electrical stimulation may have complementary benefits and the ideal implant may involve elements of both. Application of this technology to the unique anatomical limitations of the cochlea will apply similar principles but will require additional innovation as described herein.

Clinical translation and significance: The experiments described herein represent the first steps to applying a fundamentally new regenerative interface technology to the restoration of hearing. Even with the significant recent technological advances in cochlear implants, the resulting auditory experience does not approach normal hearing and central implants are even further behind. Because of these performance limitations, there are many patients suffering from hearing loss that is not adequately addressed by their hearing aids, but do not qualify for implantation. Given the ability to implant a system comprising a biocompatible construct and a plurality of auditory neurons, e.g., LEs, with minimally invasive techniques, and the overall lack of damage toneuronal tissue with implantation—it is possible that a system disclosed herein, e.g., LEs, can be implanted for patients with significant residual hearing or patients with tinnitus where hearing preservation is a requirement. In some embodiments, an improvement in the fidelity of hearing provided by implants or the ability to implant without risk to residual hearing would give options to patients who live in the gap between hearing aid and cochlear implants and would have a significant impact on their quality of life. Advanced implants for disabling deafness would benefit the population as a whole.

In some embodiments, the application of a system comprising a biocompatible construct and a plurality of auditory neurons, e.g., living electrodes, to the auditory system may increase the quality of auditory experience available from implantable hearing devices.

In some embodiments, micro-TENNs are constructs, e.g., living 3D neural constructs, that consist of a discrete neuronal population with long-projecting axonal tracts encased in a miniature tubular hydrogel (Struzyna, L. A. et al. Tissue Eng Pt A 21, 2744-2756 (2015), Cullen, D. K. et al. Tissue Eng Pt A 18, 2280-2289 (2012), Struzyna, L. A. et al. J Vis Exp Jove e55609 (2017) doi:10.3791/55609, Harris, J. P. et al. J Neural Eng 13, 016019 (2016), Struzyna, L. A et a 1. Curr Opin Solid State Mater Sci 18, 308-318 (2014), Struzyna, L. A., et al Neural Regen Res 10, 679-685 (2015)). In some embodiments, the interior of the hydrogel column contains customizable extracellular matrix (ECM) optimized to support neuronal survival and neurite extension in vitro but degrades, e.g., over several weeks in vivo. While previous attempts at artificial stimulation of the auditory system have utilized light or electrical stimulation, the disclosure provides an approach that is fundamentally different in that, in some embodiments, direct neuronal synapses are utilized to stimulate native neurons and induce auditory perception. In some embodiments, this provides a targeted and specific stimulus that is not plagued by current spread or field effects. Furthermore, the size and form factor of the constructs allows for minimally invasive implantation using stereotactic guidance, and allows all non-organic components of the system to remain external to the brain parenchyma. The constructs have been shown to be well tolerated, survive, and integrate into the native nervous system. In some embodiments, the disclosure provides use of spiral ganglion cells to fabricate micro-TENNs and apply these as “living electrodes” for stimulation of the central and peripheral auditory pathways. In some embodiments, for future applications in humans, SGN micro-TENNs may be created using induced pluripotent stem cells to, e.g., avoid the limitations of relying on harvested tissue. In some embodiments, living electrode technology provides an innovative approach to stimulation of the auditory system potentially allowing for restoration hearing that exceeds present technology.

Summary: Disabling deafness affects 5% of the world's population and represents one of the most common health concerns for US Military Veterans. Highly specific stimulation of the auditory system is not possible with today's technology and the auditory experience from implantable hearing devices is far from normal hearing. Although refinements in technology and surgical techniques have improved outcomes for cochlear implant recipients, there seems to be a limit to the performance achievable with the current designs. In some embodiments, this disclosure provides the application of living electrode technology to, e.g., the restoration of hearing via implantation of biocompatible constructs, e.g., hydrogel constructs, pre-seeded with, e.g., spiral ganglion cells into the cochlea or inferior colliculus. In some embodiments, this approach has the potential to improve hearing for patients, e.g., those suffering from hearing loss, and change how hearing loss is treated.

### Example 2: Use of a System Comprising a Biocompatible Construct and a Plurality of Auditory Neurons in an Auditory Setting

This example describes application of a system comprising a biocompatible construct and a plurality of auditory neurons, e.g., a living electrode, in an auditory setting.

### Spiral Ganglion Cell Harvest and Culture:

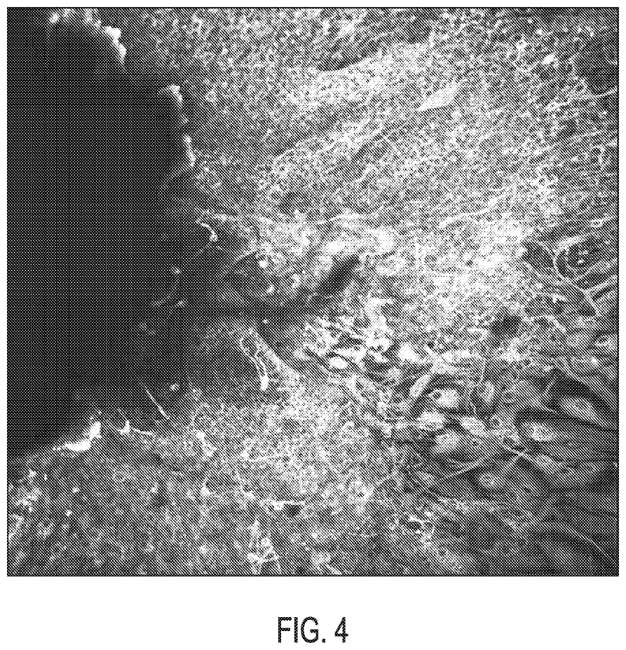

Spiral ganglion cells were harvested from the temporal bones of P0 Sprague-Dawley rat pups. To isolate spiral ganglion cells, cochleae were isolated under a stereoscope via microdissection. Following removal from the cochlea, spiral ganglia were either further separated into 200-300 μm segments for explant cultures (FIG. 4) or enzymatically dissociated following gentle micropipette trituration. For the latter, spiral ganglia were dissociated in 0.25% trypsin+1 mM EDTA at 37° C., after which the trypsin/EDTA was removed and replaced with 0.15 mg/ml DNase in HBSS. Dissociated tissue+ DNase was centrifuged for 3 min at 3000 RPM before the DNase was removed and the cells re-suspended in neuronal culture media. Neurons were plated in polystyrene petri dishes that had been immersed in PLL (0.05 mg/mL in sterile cell culture water) overnight. Dishes were then rinsed in sterile cell culture water before being immersed in laminin (20 μg/mL in sterile cell culture water) overnight prior to plating and culture. Cells were then incubated under standard cell culture conditions (37° C., 5% CO2). Half-media changes were performed every 2 days in vitro (DIV). Histology was performed as described below. Culture media consisted of Neurobasal medium, B-27 (lx), glutamax (0.5 mM), penicillin-streptomycin (20 U/mL), and BDNF (5 ng/ml). A representative aggregate is shown in FIG. 4.

To investigate the ability for distinct populations of SGN to interact, spiral ganglions were dissociated and aggregated as described above. Aggregates were then plated in polystyrene petri dishes precoated with PLL (50 μg/mL) and laminin (20 μg/mL) and allowed to grow in culture medium (described above) at 37° C., 5% CO2 with half-media changes every 48 hours. Following positive confirmation of aggregate attachment to the culture surface and axonal outgrowth via phase microscopy out to 5 DIV, aggregate cultures were virally transduced overnight to express the fluorescent calcium reporter GCaMP6 (Addgene/Penn Vector Core) (titer: 5×109 vg/mL) to enable optical imaging of neuronal activity. Following transduction, aggregates were grown and monitored for GCaMP expression/neuronal activity using a Nikon Eclipse Ti-S microscope paired with a QIClick camera and NIS Elements BR 4.13.00. Recordings were acquired at 10-15 frames per second to visualize calcium transients.

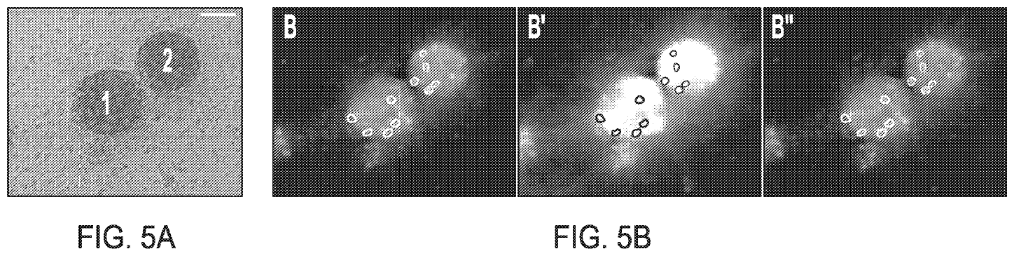

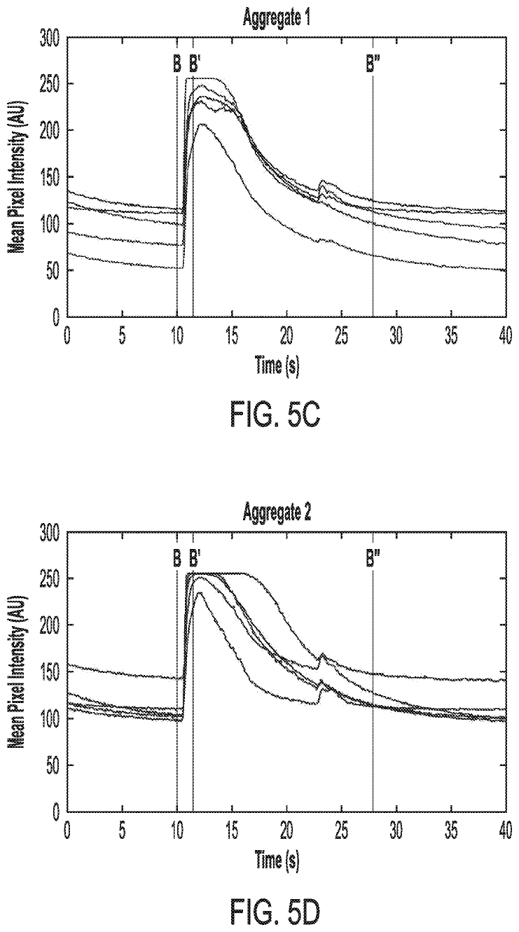

Synchronous spontaneous signaling activity was noted between the adjacent aggregates indicating functional synaptic formation between two discreet SGN populations (FIGS. 5A-5D). Similarly, synchronous spontaneous recording has been measured between SGN and centrally derived aggregates—both cortical and brainstem.

### SGN Micro-TENN Fabrication:

Micro-TENNs were constructed in a three-step process as previously shown. Briefly, 3% agarose microcolumns were molded with glass capillary tubes and acupuncture needles to the desired size (outer diameter: 398 μm; inner diameter: 160 μm) and cut to the desired length. Once formed, microcolumn channels were sterilized under UV light (30 min) and filled with ECM comprised of rat tail collagen I (1.0 mg/mL) and mouse laminin (1.0 mg/mL).

To seed cultured SNGs onto the micro-TENN, the cells were dissociated and were forced from a single-cell suspension into spheroidal aggregates via centrifugation (200×g for 5 min) in inverted pyramidal wells made of PDMS. Post-centrifugation, SGN aggregates were incubated for 48 hours in neuronal culture media before being carefully placed at one end of an ECM-laden microcolumn using fine forceps under a stereoscope. Micro-TENNs were grown in neuronal culture media with half-media replacements every 2 DIV.

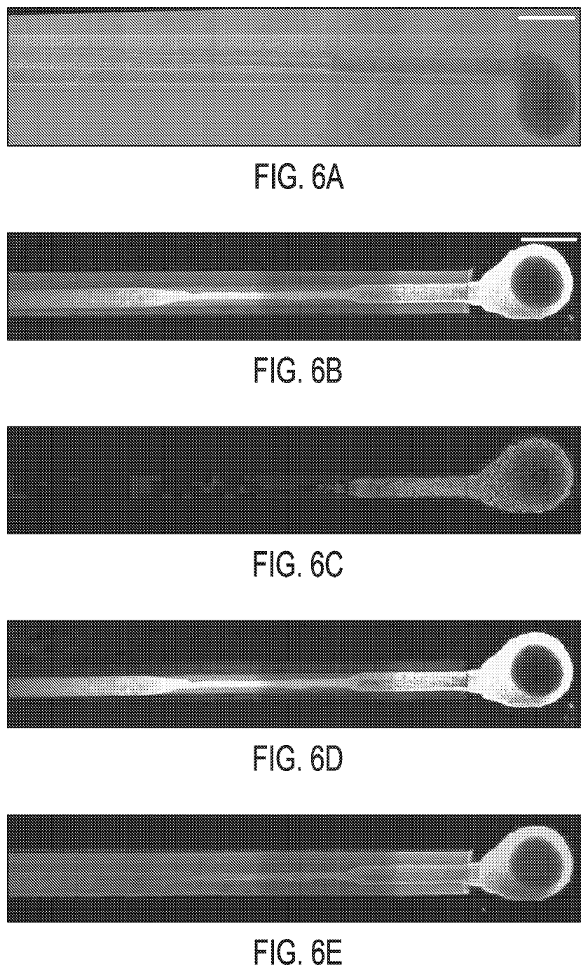

Immunocytochemistry Planar cultures and micro-TENNs were fixed in 4% formaldehyde for 35 min, permeabilized with 0.3% Triton X100+4% horse serum in PBS for 60 min, and incubated with primary antibodies overnight at 4° C. Following primary antibody incubation, samples were incubated with fluorescently labeled secondary antibodies (1:500; sourced from Life Technologies & Invitrogen) for 2 h at 18°−24° C. Finally, Hoechst (33342, 1:10,000, ThermoFisher) was added for 10 min at 18°−24° C. before rinsing in PBS. Samples were imaged on a Nikon A1RMP+ multiphoton confocal microscope paired with NIS Elements AR 4.60.00. Sequential slices of 10-20 μm in the z-plane were acquired for each fluorescent channel. All confocal images presented are maximum intensity projections of the confocal z-slices. Primary antibodies were Tuj-1/beta-III tubulin (1:500, T8578, Sigma-Aldrich) to label axons, peripherin (1:500, ab4666, Abcam) to label type II SGNs, and/or glial fibrillary acidic protein (GFAP) (1:500, ab53554, Abcam) to label astrocytes. This demonstrated that we were able to create micro-TENNs using SGN aggregates seeding within microcolumns (FIGS. 6A-6E).

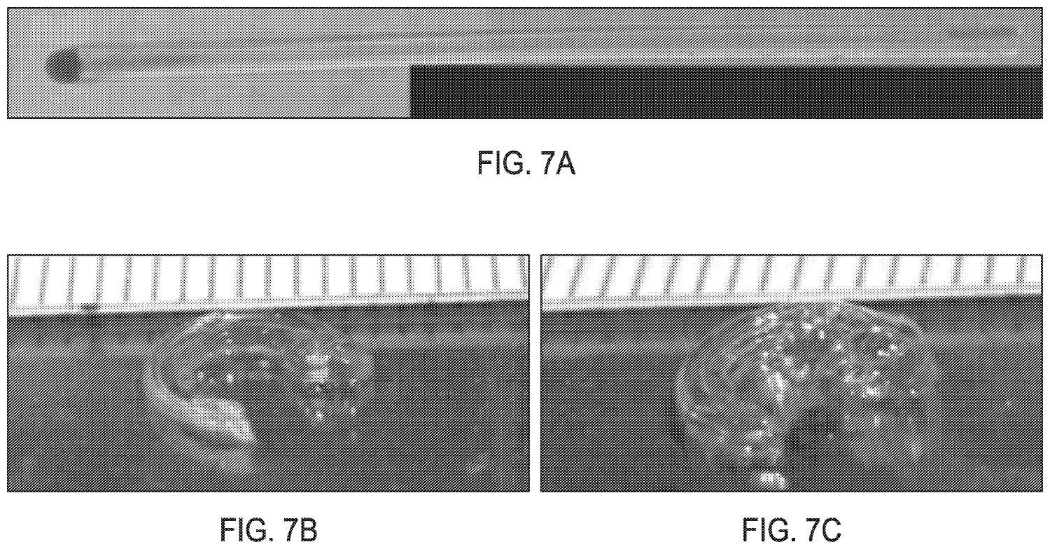

Cochlear Construct: To create constructs more amenable to implantation into the curved structure of the cochlea, a novel technique was developed. Single lumen spiral microcolumns up to 270 degrees in curvature were designed via computer aided modeling (Autodesk Inventor, Autodesk Inc.) utilizing human cochlear geometries as reference. All variants were modeled with a 1.5 mm outer diameter, 7 mm overall diameter, and 1 mm of vertical pitch. Single lumen constructs were modeled with one 850 μm diameter lumen and were 3D printed via extrusion bioprinting (BioAssemblyBot, Advanced Solutions) from Pluronic hydrogel (28.6% w/w, Advanced Solutions). Support material geometries were automatically generated as needed by the bioprinter frontend (TSIM, Advanced Solutions) and printed from the same Pluronic hydrogel prior to printing the microcolumns themselves. Print parameters were optimized to achieve continuous extrusion and minimize gapping or overprinting (nozzle pressure 55 psi, nozzle velocity 4 mm/s, line width 0.159 mm, layer height 0.05 mm, nozzle diameter 0.159 mm). In order to characterize build fidelity, constructs were printed directly onto a leveled 40×70 mm glass microscope slide (Fisher) over six minutes, measured, and imaged. As Pluronic hydrogels are opaque and thereby difficult to image via light microscopy, constructs were printed to 50, 75, and 100% completion such that internal and external dimensions could be directly validated via digital caliper (Mitutoyo) throughout. Constructs were then imaged (D3400, Nikon); 50 and 75% complete constructs are presented for reference, inclusive of deposited support material (FIGS. 7B and 7C). All printed constructs were determined to be within ±100 microns of model specifications in all dimensions listed.

Implantation: In vivo implantation of a living electrode was performed into the IC of a living rat. An adult male Sprague-Dawley rat was anesthetized and fixated in a stereotactic frame. An incision was made overlying the skull and a craniotomy performed overlying the inferior colliculus. A living electrode was inserted using stereotactic coordinates so that the terminal end was located in the central nucleus of the inferior colliculus and the cell body aggregate remained accessible at the surface of the cerebellum. The animal was healthy at three weeks and intravital multiphoton microscopy revealed viable cells in the implanted aggregate and spontaneous firing monitored via high speed fluorescent microscopy of the spontaneous activity of the GCaMP expressing neurons.

The data disclosed herein demonstrates the ability to isolate and culture SGN, maintain SNG aggregates in culture and induce interaction between distinct SGN cell populations. Further, successful axonal growth through a hydrogel microcolumn scaffold was accomplished, along with development of pre-curved constructs for cochlear implantation. Finally, implantation of a living electrode was performed into the IC with viability of both the construct and animal at 3 weeks. The SGN cells behaved similarly to previously used cortically derived neurons and were shown to interact with both centrally derived neuronal aggregates in vitro, indicating a high likelihood for success for the subsequent work. Importantly, these data demonstrate the ability to reliably create and implant living electrodes using SGN.

### Research Design and Methods

Overview and hypothesis: Current interfaces for the rehabilitation of hearing suffer from significant shortcomings. Although clinically successful, cochlear implants have relied on a similar electrode design since their inception and have only achieved about 20% of the independent channels needed to approximate normal hearing. Central implants have fared worse, achieving little more than sound awareness in most cases. One major limitation to the advancement of these technologies is the interface between the electrode and neural tissue. Through bioengineering and regenerative techniques, this disclosure provides a new technology that promises to change the nature of machine-neural interfaces for artificial stimulation of the auditory system. A hypothesis is that living electrode technology can be applied to hearing rehabilitation via integration in the cochlea and the inferior colliculus. Two distinct micro-TENN designs were developed that when seeded with spiral ganglion cells will act as living electrodes to be implanted into either the inferior colliculus or cochlea of rats. This work is based on previous work on living neuronal scaffolds and their application for restoration of central nervous system function following trauma and neurodegenerative diseases. This example utilizes a novel cell type for seeding of the scaffold (spiral ganglion neurons in place of centrally derived neurons), as well as development of a new form factor of the microcolumn scaffold designed to be placed within the cochlea rather than centrally as has been previously done. In some embodiments, one of the proposed outcomes of this work will be two biohybrid neural interfaces. In some embodiments, SGNs will be pre-grown on implantable scaffolds that are designed to be inserted centrally into the inferior colliculus, or peripherally into the cochlea. In some embodiments, both constructs will be able to direct neurites from transplanted SGN to interact with the target tissue while the cell bodies of the transplanted SNG will remain accessible for stimulation—thereby creating a so-called “living electrode” for auditory stimulation. The techniques developed in this process will also form the basis for multiple avenues of future studies into regenerative bioengineering that will be applied to expand the understanding of auditory processes and ultimately restoration of hearing.

Scientific rationale: There are no clinically available treatments to reverse damage in the cochlea for the restoration of hearing. Even as hair cell regenerative techniques improve, there are significant hurdles to overcome. The physical structure of the cochlea must be re-established and spiral ganglion neurons that have regressed must be induced to regrow and synapse with the new hair cells. Currently the most successful strategy for rehabilitation of severe hearing loss is to bypass the complex structures of the cochlea completely and stimulate the cochlear nerve directly. This approach has in many cases allowed those with significant hearing loss to once again understand speech without reading lips but falls far short of normal hearing. One of the main limitations of the current technology is positioning the implanted electrode close enough to the spiral ganglion cells to limit current spread along the cochlea and subsequent reduction in the fidelity of the transmitted signal. Numerous methods have been attempted to reduce this distance, but none have had a significant clinical impact. Direct interaction with these cells and increased selectivity of stimulation would create the potential for dramatic improvements in hearing outcomes following implantation. This disclosure combines tissue engineering and regenerative techniques to the restoration of hearing. In some embodiments, a system disclosed herein, e.g., living electrode technology overcomes several of the limitations of current options. First, in place of a standard electrode in the cochlea that is subject to current spreading, direct synaptic stimulation of native neuronal tissue is achieved in some embodiments. In some embodiments, this increases the number of independent channels available for stimulation and thus the fidelity of the auditory experience. Second, prior studies have shown the safety of implantation into brain parenchyma allowing for the possibility of enhancement of hearing without the risk of further hearing loss due to damage of the natural structures. In some embodiments, design of a construct with the complex 3D anatomy of the inferior colliculus or cochlear nucleus in mind in a well-tolerated package could result in hearing results from central implant approaching those for cochlear implantation. Finally, successful demonstration of the living electrode concept will open the door for further applications such as restoration of vestibular function or vision.

This example describes three aims to develop living electrodes for implantation into the peripheral or central auditory systems. The first aim focuses on optimization of harvest and culture of spiral ganglion neurons from embryonic or neonatal rat pups, seeding of these cells onto microcolumn scaffolds designed for implantation into either the cochlea or inferior colliculus, and delineation of optical stimulation parameters of cell aggregates. The second aim characterizes the behavior of transplanted cells in vivo. The objectives of these studies are to quantify cell survival and characterize transplanted neuronal behavior. The final aim quantifies the effects of stimulation of these transplanted scaffolds on the auditory system via electrophysiologic recordings at the auditory cortex and an innate behavioral model and compares living electrodes to standard electrode stimulation.

Bio-Fabrication Of Living Electrodes Using Spiral Ganglion Neurons And Validate Optical Control Of Their Function In Vitro. (a) Harvest and culture of spiral ganglion neurons (SGN) and evaluation of survival, synapse formation, and optogenetic transformation in vitro; and (b) in vitro seeding of SGN onto custom microcolumn scaffolds. SGN can be harvested from neonatal rat temporal bones, cultured, transduced for optical stimulation, and induced to synapse with distinct SGN and centrally derived neuronal populations in vitro. These cultured SGNs can then be grown within custom microcolumn scaffolds to form SGN living electrodes designed for cochlear or central implantation.

Spiral ganglion cells are utilized as the primary neuronal population for these studies given their natural anatomic and physiologic position in the auditory pathway. Several groups have described successful harvest and culture of spiral ganglion cells from both pre- and postnatal rodents. However, significant differences exist in the techniques used for these cultures—including whether the cells were dissociated before plating. Variables for optimization include timing of SGN harvest, culture of intact versus dissociated spiral ganglia, and duration of culture prior to seeding. All of these conditions will be tested and immunohistochemical analysis will be performed to assess cell viability, proportion of the neuronal cells-of-interest in culture (versus supporting cells), and tendency for neurite extension. Viability will be evaluated with calcein AM (labeling live cells) and ethidium homodimer (labeling the nuclei of dead cells), and neurite extension will be evaluated with immunohistochemistry (using antibodies recognizing axonal cytoskeletal constituents).

SGC harvest and culture: To isolate spiral ganglion cells, cochlea will be isolated from Sprague-Dawley rats at specific embryonic and neonatal timepoints (E18, P0, P5, P10) under stereoscope via microdissection. Following removal from the cochlea, spiral ganglia will either be further separated into 200-300 μm segments for explant cultures or enzymatically dissociated following gentle micropipette trituration. For the latter, spiral ganglia will be dissociated in 0.25% trypsin+1 mM EDTA at 37° C., after which the trypsin/EDTA will be removed and replaced with 0.15 mg/ml DNase in HBSS. Dissociated tissue with DNase will be centrifuged for 3 min at 3000 RPM before the DNase will be removed and the cells re-suspended in neuronal culture media. Neurons will be plated in polystyrene petri dishes that has been immersed in PLL (0.05 mg/mL in sterile cell culture water) overnight. Dishes will then be rinsed in sterile cell culture water before being immersed in laminin (20 μg/mL in sterile cell culture water) overnight prior to plating and culture. Cells will then be incubated under standard cell culture conditions (37° C., 5% CO2). Half-media changes will be performed every 2 DIV. As described in Preliminary Data, culture media consists of Neurobasal medium, B-27 (lx), glutamax (0.5 mM), penicillin-streptomycin (20 U/mL), and BDNF (5 ng/ml). A similar technique to the dissociated protocol described here is used for the harvest and culture of centrally derived neurons, except in that case brain cortical tissue is collected rather than spiral ganglion tissue.

Different media formulations for SGN cultures have been reported in literature; further optimization will involve direct comparisons of growth, neuronal survival, and function between SGN cultures and/or constructs grown in different media. BDNF has been shown to promote SGN survival—both in vitro and in vivo—and has been implicated in potential functional reinnervation (69, 70). However, the benefits of BDNF are dependent on both the concentration and the age of the SGNs as determined by the embryonic/postnatal isolation timepoint and may predominantly affect type II vs type I SGNs. In addition to culture media and conditions, in some embodiments, the aggregation protocol will need optimization to determine the best centrifugation speed, duration, and post-centrifuge incubation period for SGN constructs. Additionally, microcolumns may be seeded with either aggregates or similarly sized spiral ganglion explants; how the choice affects construct growth and function is an open question.

To test whether axo-axonal interactions are sufficient for physiologic coupling of distinct SGN aggregates, following spiral ganglia dissociation and aggregation as previously described, spiral ganglion aggregates will be seeded within agarose microcolumns to generate bidirectional (i.e. 2-aggregate) micro-TENNs. Aggregates will also be plated in 2D cultures as a control for potential growth constraints within the micro-TENN architecture. Following culture of at least 7 DIV, micro-TENNs and control aggregates will be fixed with formaldehyde for fluorescent immunolabeling. Selected antibodies will target pre-synaptic (synaptophysin) and post-synaptic (PSD-95) markers, as well as neuronal somata and axons (beta tubulin-III). Immunolabeled samples will be imaged using confocal or two-photon microscopy, with axo-axonal synapses identified through overlap of the synaptic markers with axons. Further validation of axo-axonal synapses may be demonstrated through the immunolabeling of dendrites (e.g. with MAP-2) to further differentiate axo-axonal from axo-dendritic and axo-somatic synapses.

Optogenetic transformation: Two optogenetic transformations will be performed, one for report monitoring of activation, and one to allow for optical stimulation. For calcium imaging, SGN will be transduced with an AAV vector carrying the GCaMP6 gene (Penn Viral Vector Core) for 16-24 hours. A full media change will then be performed to remove remaining virus particles. Beginning 4-5 days after viral transduction, calcium activity can be observed using a standard fluorescence or confocal microscope. This activity will then be recorded as videos using microscope software (NIS-Elements, Nikon). Fluorescence activity will then be measured using manually drawn regions of interest in NIS-Elements or automated Matlab scripts. For optogenetic stimulation, SGN will be transduced with an AAV vector carrying a light-activated opsin gene (e.g., ChR2, CrimsonR) (Penn Viral Vector Core) at a titer of 1.34×10¹⁰ genome copies/mL for 16-24 hours. Stimulation experiments are delayed approximately 2 weeks after viral transduction to allow for maturation of the opsin proteins. Optical stimulation will be performed using an LED array consisting of appropriate wavelength LED coupled to 200 μm optical fibers (Thorlabs). This system is driven by a multi-channel LED driver (Thorlabs) and digital acquisition board (National Instruments). Stimulation parameters are defined below. The cells induced with Ca²⁺-dependent optical activity reporters can be used to monitor for neuronal activation in real time, while cells with light-activated opsins can be stimulated using light in place of electrical stimulation (see example in FIGS. 2A-2F).

The technique disclosed herein relies on the ability for the harvested SGNs to interact with and stimulate native SGNs in the host cochlea. The ability for SGN-SGN interaction and stimulation has not been previously reported. It has been shown that peripherally derived neurons can be induced to perform this behavior (e.g., dorsal root ganglia neurons synapsing with other dorsal root ganglia neurons), and synchronous passive neuronal activity between adjacent SGN populations has been observed. To further test this, one SGN population will have Ca²⁺-dependent optical activity induced, while another will have light-activated opsins. The two populations will be induced into spheroidal aggregates (described below) and cultured in proximity to one another. Axonal outgrowth will be monitored. Once it is apparent there is interaction between the two populations, Ca²⁺-dependent optical activity will be monitored from the first group as the opsin population is stimulated optically. Confirmation of Ca²⁺-dependent optical activity will indicate interaction between the two populations and the ability for axons of transplanted SGN to induce activity in native SGN in vivo. A similar experimental design will test the ability of SGN to interact with and induce activity in a centrally derived population as an indicator of the ability for transplanted SGN to stimulate native neurons in the inferior colliculus (thus better representing the in vivo circuitry).

Optical stimulation parameters: Although previous studies have evaluated the response of in vivo transduced neurons of the auditory system to optogenetic stimulation, it is possible that the cell aggregates formed for the creation of LE perform differently than transduced native neurons. To evaluate this, aggregates will be cultured in proximity to each other as above. Optical stimulation patterns derived from previous optogenetic testing of the auditory system will then be performed to establish thresholds for activation, as well as frequency and intensity response curves. Stimulation of the opsin transduced aggregate will be via 473 nm pulses of light applied via optical fiber as above located 2 mm from the aggregate of interest. Light intensity will be calibrated by positioning the fiber 2 mm from a high-sensitivity thermophile sensor connected to a power meter. Stimulation will be varied between 1-20 uJ/mm 2 exposure, 0.1 to 10 ms pulse duration, and 0-70 hz frequency while recording light emittance from the GCaMP derived aggregate. Response curves will be generated to determine the threshold (10% above background) and maximal values for each of the parameters of interest. This testing will be repeated for all combinations of SGN and central neurons (cortical and brainstem) as stimulated and reporter aggregates. Controls will be performed via similar culture conditions with omission of opsin transduction in the stimulated aggregate.

It is expected that the experiments described herein will be able to optimize harvest and culture conditions that will provide consistent, high-quality populations of SGN. Further, it is anticipated that co-culture of separate populations of these neuronal populations in proximity will induce axon elongation and synapse formation between them that can be detected via Ca²⁺-dependent optical activity. This may suggest that transplanted SGNs have the ability to synapse with native SGN in living rat cochlea. Similar behavior is anticipated when co-culturing SGN with centrally derived neurons in anticipation of central implantation. Subsequently, the latter pairing will be reversed to test the ability for centrally derived neurons to induce depolarization in SGN. This may allow for increased yield of constructs since the number of central neurons that can be obtained from a given animal far exceeds that of SGN. Further, utilization of optical stimulation for activation of spiral ganglion cells has been established with predictable responses to variations of the input stimuli. It is expected that ChR2 induced neuronal aggregates may exhibit similar stimulation parameters given successful optical stimulation of the auditory pathways previously published.

Following successful completion of the above steps, SGN populations will be seeded within microcolumn scaffolds. Two custom hydrogel scaffolds will be developed. In some embodiments, the first will be utilized for implantation into the inferior colliculus and will be similar to those previously used. This scaffold will allow the neurites of the implanted SGN to synapse in the inferior colliculus, while their cell bodies remain on the surface and are accessible for recording or stimulation. In some embodiments, the second will be similar but will also conform to the curvature of the cochlea and will guide the SGN axons towards native spiral ganglion cells running through the modiolus of the cochlea.

Construct Design: In some embodiments, the scaffold comprises 3D hydrogel microcolumns that are designed to guide the extension of neurites from transplanted SGN to interact with native neuronal tissue—while the cell bodies of these SGN remain accessible. The microcolumn comprises 3% agarose with a collagenous ECM on the interior to allow for neurite adhesion and extension). To fabricate these scaffolds, agarose will be dissolved in heated Dulbecco's phosphate-buffered saline (DPBS). A mold of the appropriate shape for the external dimensions of the scaffold will then be created and thin needles will be used to create the space that will become the lumen of each microcolumn. This mold will then be filled with the dissolved agarose and allowed to gel. The mold will then be removed leaving an agarose shell with a hollow bore. The microcolumn will then be sterilized via exposure to UV light for 15 minutes. The microcolumns will then be filled with a collagen ECM solution (0.2-2.0 mg/mL rat-tail collagen type I in neuronal growth medium). The collagen will then be allowed to polymerize prior to seeding with cultured cells in a tissue culture incubator (37° C., 5% CO2). From previous work constructing hydrogels, it was noted that the physical properties of the scaffolds allow for deformation without fracture or buckling allowing for some conformation to the shape of the cochlea beyond the pre-formed shape. In some embodiments, additional customization of construct design will utilize 3D printing technology.

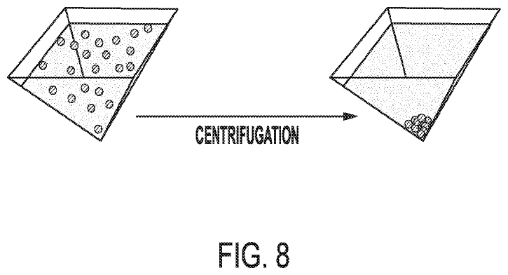

SGN seeding onto custom scaffolds: To seed cultured SGNs onto the microcolumns, the cells will be dissociated and forced from a single-cell suspension into spheroidal aggregates via centrifugation (200×g for 5 min) in inverted pyramidal wells made of PDMS (FIG. 8). Post-centrifugation, SGN aggregates will be incubated for 48 hours in neuronal culture media before being carefully placed at one end of an ECM-laden microcolumn using fine forceps under a stereoscope. The resulting constructs will then be grown in neuronal culture media as above (with half-media replacements every 2 DIV) to form SGN living electrodes.

Given the unique physical constraints of the cochlea, several scaffold shapes will be created and will be tested on harvested adult rat cochlea with various implantation techniques. In vivo insertion of the microcolumns will be performed to simulate insertion into living animals, and the cochlea will be histologically analyzed for location of placement, and damage induced on insertion. Areas of optimization for the living electrodes include diameter and shape of the microcolumns, techniques for 3D printing custom microcolumn shapes, type and concentration of ECM filling the microcolumns, seeding density of SGNs, and culture timing. The portion of this construct housing the SGN cell bodies will remain in the bulla of the temporal bone for cochlear implantation and on the brainstem surface for IC implantation, allowing for access for recording and stimulation.

Once the final configurations for the microcolumns have been established, SGN will be seeded and allowed to grow for 1, 2, 4, 7, and 14 DIV before histological analysis. Number of neurites, neurite length, and cell viability will be evaluated at the given timepoints. The constructs will then be fixed in 4% formaldehyde and permeabilized with Triton X. The tissue will be stained with antibodies specifically to evaluate axonal growth including MAP-2, a microtubule associated protein found in dendrites, β-tubulin III, a microtubule element enriched in axons, and glial fibrillary acidic protein, an intermediate filament protein expressed in astrocytes as previously described. To characterize growth in the SGN constructs, phase images of SGN living electrodes will be taken using a Nikon Eclipse Ti-S microscope paired with a QIClick camera and NIS Elements BR 4.13.00. The longest neurite will be manually identified in each phase image using ImageJ (National Institutes of Health, MD). Growth rates will be quantified as the change in length of this longest process at each timepoint divided by the number of DIV from the preceding timepoint. All length measurements will be standardized as the longitudinal distance of the endpoint of the longest process relative to the edge of the source aggregate (identified at 1 DIV).

SGN living electrode creation for the inferior colliculus is similar to those previously created for application in the nervous system. In some embodiments, it is expected that the cochlear living electrode can be created in a manner to allow for conformation to the anatomy of the cochlea—through a combination of pre-designed shape and flexibility of the construct. In some embodiments, harvested SGN is anticipated to seed onto scaffolds with high viability and show neurite extension at rates similar to previous studies with centrally derived neurons.

### Demonstration of Delivery, Survival, and Integration of Living Electrodes In Vivo.

In vivo implantation of (a) cochlear and (b) central SGN living electrodes and evaluation of survival and integration. In some embodiments, SGN living electrodes can be implanted into rat cochlea or inferior colliculi and the transplanted neurons can survive and synapse with appropriate target neurons.
Implantation: Following successful seeding of the microcolumns and sufficient evidence for SGN viability and neurite extension, the optimized living electrodes as described above will be implanted into living rats. For cochlear implantation, the scaffold will be inserted via the round window of the cochlea of anesthetized rats (or alternative approach to the cochlea as below) similar to previously described protocols with more standard electrodes. Rats will be anesthetized with isoflurane and the hair behind one ear will be removed and the area cleaned with betadine. A postauricular incision and dissection of the underlying musculature will expose the auditory bulla of the temporal bone. Utilizing an operative microscope, the bulla will then be removed with a drill and the round window exposed. It is expected that a round window insertion will be performed. If so, the round window niche will be removed, and the round window membrane incised. The construct will then be gently advanced through the round window until resistance is encountered or the construct is fully inserted. The neuronal cell body aggregates will remain accessible in the auditory bulla. The postauricular skin incision will then be closed.

For inferior colliculus insertion, a rat will be anesthetized with isoflurane and mounted in a stereotactic frame. The head will be shaved and cleaned with betadine, after which an incision and small craniotomy will be made over the inferior colliculus. SGN living electrodes will then be drawn into a Hamilton syringe mounted onto the stereotactic frame and will be delivered via needle injection, after which the Hamilton syringe will be immobilized while the needle will be slowly raised to deliver the constructs with minimal force. Again, the neuronal cell body aggregates will remain on the surface of the brain allowing for later optical stimulation. Following needle removal from the brain, the craniotomy will be sealed with bone wax and the skin sutured. For both protocols, rats will be given meloxicam (2.0 mg/kg) and post-operatively monitored until recovery. This general procedure is routinely performed.

Following implantation, the animals will be allowed to recover from surgery and survived for 1, 2, 4, 8, or 12 weeks post-implantation, at which time their brains or temporal bones will be harvested. To evaluate for the ability of LE to monitor physiologic activity, prior to euthanasia and harvest of tissue, intravital fluorescent microscopy will be performed to quantify spontaneous GCaMP activity from the IC implanted electrodes. Rats will be anesthetized and secured via palate bar in a stereotactic frame. Fluorescent microscopy will then be performed as above. Utilizing GCaMP induced living electrodes for monitoring of endogenous neuronal activity is as previously performed, and was utilized for the successful IC implant described above. Anesthetized animals will also be presented with wide-band auditory stimuli during monitoring of the living electrode cell bodies. In some embodiments, increase in activity following acoustic stimuli will confirm physiologic interaction with native neuronal populations in the auditory pathway and the timing of activation will be compared to known timing of ABR signals from the IC. The degree of stimulation recording will be compared to histological evaluation from each time point which may allow for non-invasive monitoring of synapse formation in future studies. For histological analysis, animals will be transcardially perfused with heparin saline and then 10% neutral buffered formalin. Extracted brains or temporal bones will be rinsed in PBS and then immersed in 30% sucrose. Following flash freezing in 2-methylbutane, the brains will be stored at −80° C. or immediately sectioned (20 μm thickness). For cochlear histology, demineralization and fixation of the cochlea will be performed as previously described. For immunohistochemistry, the tissues will be fixed in 4% formaldehyde for 35 min, permeabilized with 0.3% Triton X100+4% horse serum in PBS for 60 min, and incubated with primary antibodies overnight at 4° C. Following primary antibody incubation, samples will be incubated with fluorescently labeled secondary antibodies (1:500; sourced from Life Technologies & Invitrogen) for 2 hours at 18°−24° C. Finally, Hoechst (33342, 1:10,000, ThermoFisher) will be added for 10 min at 18°−24° C. before rinsing in PBS. Samples will be imaged on a Nikon A1RMP+ multiphoton confocal microscope paired with NIS Elements AR 4.60.00. Sequential slices of 10-20 μm in the z-plane will be acquired for each fluorescent channel. All confocal images presented will be maximum intensity projections of the confocal z-slices. Primary antibodies will be Tuj-1/beta-III tubulin (1:500, T8578, Sigma-Aldrich) to label axons, peripherin (1:500, ab4666, Abcam) to label type II SGNs, and/or GFAP (1:500, ab53554, Abcam) to label astrocytes.

Living electrodes will be implanted in either the cochlea or inferior colliculus of living rats. Histological analysis will be performed at 1, 2, 4, 8, and 12 weeks, and each timepoint will be replicated 10 times. Two additional rats per timepoint will act as controls by implanting acellular hydrogel constructs. Five additional rats will be used for testing of alternative cochlear implant approaches. Therefore, it is estimated that 110 rats (2 groups×5 timepoints×10 replications+5 controls+5 cochlear implant alternatives) will be used over the course of the grant period.

In some embodiments, it is expected that implantation of the SGN living electrodes into the inferior colliculus will lead to successful axonal extension and synapse onto native cells. This technique is similar to that performed previously. As disclosed above, implantation into the IC has already been accomplished with viability of both the animal and implanted neurons at three weeks. Though technically different than central implantation, in some embodiments, it is expected that transplanted SGN via the cochlea will similarly survive and extend neurites to synapse on native spiral ganglion cells. Further, cell viability and axonal growth rates are expected to be similar to previous studies.

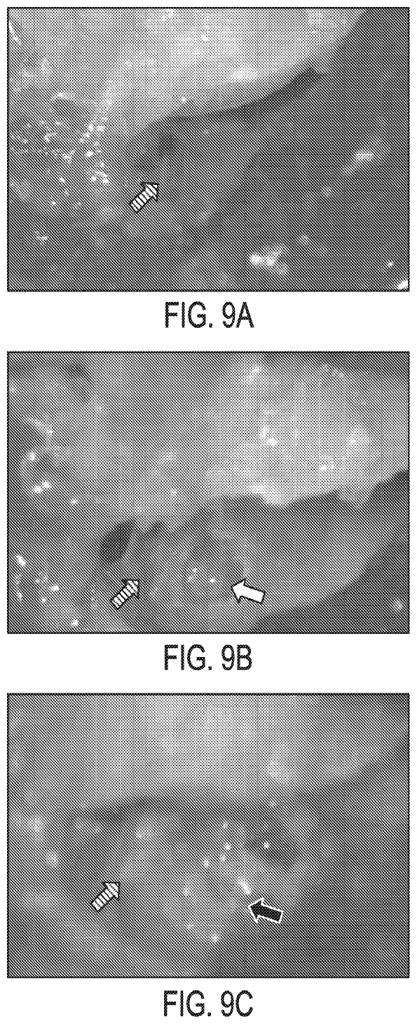

Should direct axonal interaction in the scala tympani still not be obtainable, in some embodiments, direct access to the cell bodies of the spiral ganglion can be achieved through multiple cochleostomies as described above, or by complete removal of the superficial portion of the otic capsule of the cochlea (FIG. 9). In some embodiments, while this method can destroy the natural structure of the cochlea, it can potentially allow direct access to the entire length of the spiral ganglion. It would also potentially allow for access to the cochlear nerve itself as it enters the cochlea. Although these techniques are more applicable to non-human implantation, demonstration of interaction with native SGN by implanted living electrodes will greatly advance the technology while less invasive implantation techniques can be developed. As disclosed above, axo-axonal interactions in cultured SGN populations has already been established. In some embodiments, it is also possible that the SGN do not remain viable after seeding onto the scaffolds, or do not show appropriate neurite extension, in this case, neurotrophic factors may be added to the ECM used to grow the axonal tracts. In some embodiments, if the SGN cannot be induced to grow effectively, centrally- or peripherally derived neurons may be used in their place. In some embodiments, if native SGN axons can be induced into the scala tympani, but the physical constraints of the cochlea prevent construct insertion through the round window, an extended round window or cochleostomy approach will be performed. In these cases, either an anterior-inferior extension of the round window will be drilled, or a completely separate anterior-inferior cochleostomy will be performed. In some embodiments, if the construct is appropriately placed, and the SGN remain viable, but there is no evidence of extension of axons along the cochlea with synapse onto SGN, centrally derived neurons may be utilized in place of SGN. In some embodiments, alterations in the composition of the ECM included in the living electrode lumen, or introduction of neurotrophic factors may also induce improved synaptic interactions.

### Assessment of Efficacy of Living Electrodes to Transduce Auditory Inputs to the Brain.

Neuro-electrophysiologic and behavioral evaluation of stimulation of implanted SGN living electrodes. In some embodiments, after implantation and integration of SGN living electrodes in the cochlea or inferior colliculi of rats, either electrical or optical stimulation of transplanted neurons can lead to detectable signals in the auditory cortex, and measurable behavioral changes indicating influence on the auditory pathway.

Electrophysiologic recording: Initial confirmation of the ability for transplanted SGN living electrodes to generate signals in the auditory cortex will be completed in deafened rats. Single-channel constructs will be implanted into the cochlea or inferior colliculus as described above and allowed to integrate for the optimal time disclosed above. At the time of electrophysiologic recording, a rat will be anesthetized with isoflurane and deafened by direct bilateral cochlear irrigation of the selectively cochleotoxic antibiotic Neomycin. Deafness is verified by the absence of a monaural click-evoked auditory brainstem response (ABR) (83) at 93 dB peak equivalent sound pressure level in both ears. The animals head will then be secured in a stereotactic frame. The area over the implanted SGN living electrode will be exposed (via reopening of the craniotomy incision for IC, and the postauricular incision for cochlear) and a second craniotomy overlying the auditory cortex will be made. The exposed cell bodies of the transplanted SGN will be stimulated optically and recordings will be made from the auditory cortex. Optical stimulation parameters will be tested similarly to those described in Aim 1 and stimulus-response comparisons will be made via recording at the auditory cortex. To compare the LE stimulation to standard electrode stimulation, a single channel Teflon-insulated platinum/iridium wire with the distal 1 mm of insulation removed will either be placed in the IC via stereotactic coordinates as above or into the cochlea via the round window or cochleostomy approach. Stimulation parameters will be guided by previously published similar studies. Briefly, amplitude thresholds will be defined as the minimal amplitude able to generate reproducible signal in the auditory cortex. Initial parameters will be 100 microsecond biphasic pulses with and interpulse interval of 100 microseconds. The current will be started at 0.1 mA and increased by 0.1 mA steps until reliable signal can be detected. Once the threshold amplitude levels have been established, the frequency and pulse width will be adjusted, and stimulus-response curves will be generated. This will allow for direct comparison of living and standard single channel stimulation at the cochlea or IC. For all studies, electrophysiological signals will be continuously recorded at 32 kHz using an Intan 128ch stimulating/recording system (Intan) from a planar 32-channel four-pronged probe (Neuronexus) placed in the auditory cortex via stereotactic coordinates. Wideband signals will be used for analysis of local field potentials (LFP). For initial spike detection, the wideband signals will be passed through a digital high-pass filter (0.6-6 kHz) online. Spike sorting will be performed offline using KlustaKwik and SpikeSort 3D software (Neuralynx), followed by manual adjustment of the clusters. Subsequent cluster analysis will be performed using custom Matlab scripts.

Behavioral response: The electrophysiologic recordings above will confirm that activation of the auditory pathways is possible with the living electrode techniques, however it does not give insight into the auditory experience induced by such activation. In order to further test the adaptation of living electrodes into the auditory system, elicitation and modification of the acoustic startle reflex (ASR) will be investigated. The ASR is an innate behavioral response in rodents to sudden loud acoustic stimuli that requires no training and has been well established as a model for the study of central auditory pathways. This process can be readily measured without the need for complicated tasks or behavioral training. The ASR can be reliably attenuated by preempting the acoustic stimulus with lesser stimuli—either acoustic or via electrical stimulation of the auditory pathways—known as prepulse inhibition (PPI). To evaluate if living electrodes can elicit these responses, the ASR will be induced with stimulation of the cochlea with living electrodes, and the ability for living electrode simulation of the cochlear or IC implant to modify the PPI will be evaluated.

At the time of living electrode implantation as above, a custom head-fixation device will be attached, as previously described, with modifications to avoid coverage of the area overlying the IC or cochlear implantation site. The implant will be allowed to integrate for the appropriate time as disclosed above. For testing, an animal will be placed in a custom restraining rig mounted on a continuous force transducer that was previously developed. This rig allows for firm fixation of the head of an awake animal while continuous recording of the downward force exerted by the animal is monitored. The rig is housed in a sound isolation booth with calibrated speakers for introduction of auditory stimuli and access for fiberoptic wiring for optical stimulation of implanted living electrodes. Unimplanted animals will be tested to validate the experimental setup and establish baseline recordings. Animals implanted with either cochlear or IC living electrodes will then be tested for the ability for stimulation of the living electrode to modulate prepulse inhibition of the ASR induced by auditory stimulus. For both groups, the stimulus will be presented to the contralateral ear. It has been shown that the location of the PPI stimulus relative to the acoustic stimulus does not affect the degree of PPI. The optic stimulation parameters will be adjusted within the parameters defined above to develop a stimulus-response relationship for PPI. Further, similar studies will be performed with single channel standard electrodes as above and comparison will be made between the living and standard electrodes. Finally, the ability for stimulation of the cochlear standard and living electrode to induce the ASR will be evaluated. Stimulation parameters will be defined as above with increases in amplitude until the startle response is elicited. There is no published data on the ability for non-auditory direct stimulation of the cochlea to elicit or inhibit the ASR. In some embodiments, stimulation of the IC in not expected to trigger the ASR as the neuronal arc for this reflex does not require the IC, however electrical stimulation of the IC has been shown to modulate PPI of the ASR.

The behavioral protocols were previously developed and central auditory processing in rodents has been previously reported. Recording from central auditory pathways, designing and interpreting auditory-based behavioral animal models, and processing neural recoding data was also previously disclosed.

Animal numbers: Rats undergo electrophysiologic and behavioral recordings after integration of living electrodes into either the cochlear or inferior colliculus. For the electrophysiologic recordings, it is estimated that 20 implanted rats with 10 controls per group will be required. An additional 5 rats will undergo similar recording set-ups without having been implanted with living electrodes to validate the set-up these same animals will then be implanted with standard electrodes to perform the comparison studies outlined above. Similarly, for behavioral analysis, 20 implanted rats with 10 controls, and 5 un-implanted rats will be used. Thus, 130 rats will be used in for these experiments.

The experiments disclosed herein are expected to establish the efficacy of living electrode stimulation at either the cochlea or IC to induce reliable and predictable signal changes in the auditory cortex. Further, fundamental behavioral analysis of the activation of the ASR, and its inhibition via prepulse inhibition will be evaluated. Statistical analyses: Across all experiments described herein, the minimum number of animals necessary for scientific validity will be used, and animals will be used for multiple experiments as appropriate. To estimate the group sizes necessary to obtain significant differences (where differences exist), a power analysis was performed (power=0.90, α=0.05). For all data, we will examine normality, and adjustments will be made for non-normal data. As appropriate, ANOVA will be performed with SGN living electrode type, treatment, and time as independent variables and outcome measures (previously described) as dependent variables. When differences exist between groups, post-hoc pair-wise comparisons will be performed. For all statistical tests, p<0.05 will be required for significance.

Conclusion: Clinically significant advances in hearing restoration is expected to require novel bioengineering and regenerative medicine techniques. Described herein is the application of living electrode technology to this important clinical need.

Scientific Advances: Although the experiments as described are primarily intended for the development of living electrode implants for auditory rehabilitation, this work will also form the foundation for multiple avenues of continued investigation. This work will allow for the development of a research plan to perform comparative effectiveness preclinical and translational evaluations of multiple approaches to peripheral and central auditory rehabilitation—including neural engineering, surgical techniques, functional outcomes measures, behavior, neurophysiology, and histology. This experimental framework can be deployed to innovate and vet future technologies in the decades to come.

More specifically, the advanced tissue engineering techniques that are developed can, in some embodiments, be deployed to create a biofidelic testbed to systematically evaluate other auditory therapies and potentially reduce the reliance on more time and resource intensive in vivo models. In some embodiments, one possible manifestation of this is a modular and customizable ‘in vitro cochlea’ comprised of multi-cellular living electrodes. In some embodiments, this would include an SGN aggregate in the center with axonal extensions to a centrally derived aggregate on one end, and cultured cochlear hair cells on the other. In some embodiments, the three initial cell types of the auditory pathway are represented, and each can be studied and manipulated independently, as well as the connections between them. In some embodiments, although the complex anatomy to transduce sound would be lacking, multiple important lines of inquiry could be investigated: electrical stimulation of distal axons/SGN cell bodies as it relates to cochlear implants, regenerative techniques for hair cells or SGN, computational models of signal processing in the cochlea, ototoxic and otoprotective medications, and many more. In some embodiments, as construct fabrication techniques advance, this model will continue to evolve to more closely approximate a living cochlea. In some embodiments, an additional technique that will be advanced during the execution of this project is the use of living electrodes as monitors of physiologic neuronal activity. In some embodiments, living electrodes are biologic implants that can be functionalized as light-emitting monitors—as described above. Further refinement promises to open multiple new avenues for investigation of central auditory pathways without the limitations currently imposed by traditional electrode technology. Finally, these in vitro and small animal techniques can feed into the ongoing primate models which are capable of advanced behavioral training, and eventually human clinical testing.

In some embodiments, additional advances in the culture and evaluation of SGN is expected to be made. This is an important cell type for multiple auditory pathologies and will prove to be critical to the advancement of several approaches to the restoration of hearing. In some embodiments, as mentioned above, the effective outgrowth of SGN axons into the scala tympani would have the potential to dramatically improve the performance of existing cochlear implant technology. In some embodiments, the techniques described above will help to further these studies by introducing a modality combining both growth factors and ECM proteins—each of which has independently shown promise. Second, it has been shown that hair cell loss in turn leads to loss of spiral ganglion cells. In some embodiments, regenerative cochlear hair cell techniques will rely on the ability for SGN to regrow axons in order to be utilized by the auditory pathways and regenerative SGN techniques will require both the interaction with hair cells and integration into the central auditory pathway. Finally, hidden hearing loss and synaptopathy are increasingly recognized causes of deafness but are currently poorly understood. The direct study of SGN in vitro and the ability to study the interactions of SGN with both hair cells and brainstem neurons in an in vitro cochlear model as described above could pave the way for improved diagnostic and treatment modalities. In sum, the work described here promises to contribute to the advancement of hearing science far beyond the development of living electrode implants.

Future Directions: In some embodiments, the proposed work will form the foundation for two novel living electrodes for auditory rehabilitation. In some embodiments, one is meant to be implanted peripherally in the cochlea, and one is to be implanted centrally into the inferior colliculus. The methods presented here will result in single-channel implants, however, in some embodiments, to improve clinical applicability, multi-channel implants will be needed, and the configuration must be customized to the morphology of the target tissue. In some embodiments, multi-channel implants will be based on nano-3D printed constructs utilizing the NanoScribe Photonic Professional GT (Nanoscribe GmbH, Eggenstein-Leopoldshafen, Germany) which is a 3D printing system capable of smaller than micrometer features.

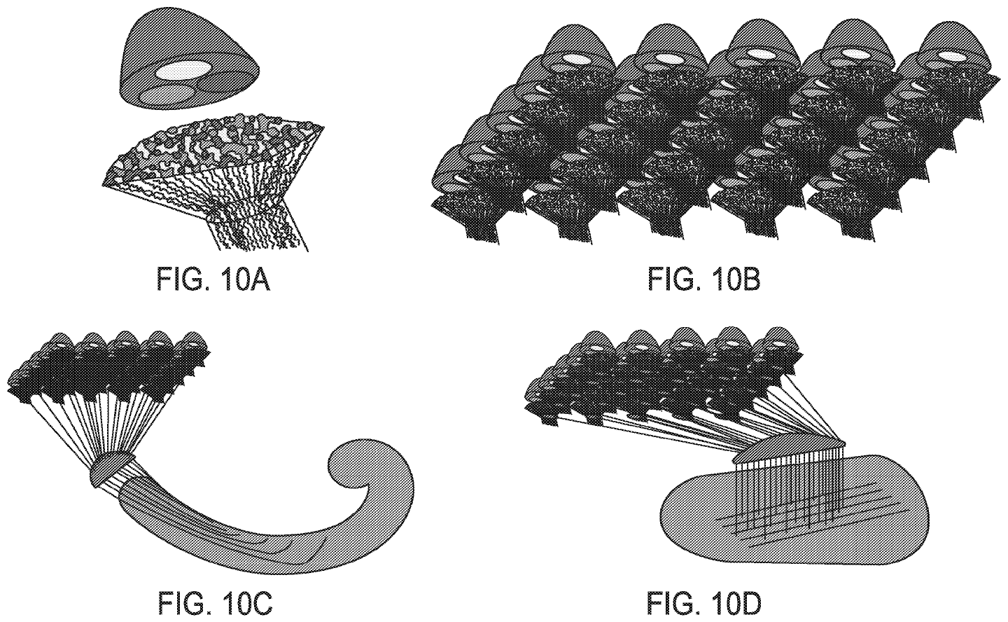

In some embodiments, the IC and cochlear designs will share a common element for the distribution of neuronal cell body aggregates and optrode interface but will differ in the conformation of the axonal channels. In some embodiments, for the common elements, neuronal cell body aggregates will be spaced such that each aggregate can be stimulated by a triad of optrodes transmitting light at distinct wavelengths. In some embodiments, each neuronal cell body aggregate will contain cell populations of optogenetically transformed cells to respond to each of these wavelengths of light. In some embodiments, a two-dimensional array of optrode triads will be paired with a similar array of neuronal cell body aggregates. In some embodiments, the channels emanating from the underside of the cell body aggregates will be designed to distribute axons throughout the three-dimensional volume of the inferior colliculus, or along the length of the cochlea. In some embodiments, even when fully implanted, the optrode/aggregate portion of the construct will sit on the surface of the brainstem or in the auditory bulla outside of the cochlea (FIG. 10). In some embodiments, no non-organic portions of the implant will be introduced into the parenchyma of the brainstem or need to conform to the structure of the lumen of the cochlea.

### ENUMERATED EMBODIMENTS

The following enumerated embodiments are provided, the numbering of which is not to be construed as designating levels of importance.
Embodiment 1 provides a system comprising:
(a) a biocompatible construct comprising a first end, a second end, and a body; and
(b) a plurality of auditory neurons.
Embodiment 2 provides the system of embodiment 1, wherein the biocompatible construct comprises an inner surface and an outer surface.
Embodiment 3 provides the system of embodiments 1-2, wherein the inner surface of the biocompatible construct defines a luminal core.
Embodiment 4 provides the system of embodiments 1-3, wherein the outer surface of the biocompatible construct comprises at least one hydrogel.
Embodiment 5 provides the system of embodiments 1-4, wherein the hydrogel comprises a hydrophilic biopolymer and/or a synthetic polymer.
Embodiment 6 provides the system of embodiments 1-5, wherein the hydrogel is at least partially cross-linked, wherein the cross-linking optionally increases stiffness, reduces porosity, and/or increases degradation time.
Embodiment 7 provides the system of embodiments 1-6, wherein the hydrophillic biopolymer comprises one or more of agarose, hydrogel, hyaluronan, chitosan, alginate, collagen, dextran, pectin, carrageenan, polylysine, gelatin, hyaluronic acid, fibrin, and methylcellulose.
Embodiment 8 provides the system of embodiments 1-7, wherein the hydrophillic biopolymer comprises agarose.
Embodiment 9 provides the system of embodiments 1-8, wherein the agarose is at about 0.25-30%, about 0.25%-3%, about 0.5%-3%, about 1-20%, about 1.5-10%, about 2-9%, about 2.5-8%, or about 3-7%.
Embodiment 10 provides the system of embodiments 1-9, wherein the agarose is at about 3%.
Embodiment 11 provides the system of embodiments 1-10, wherein the hydrophillic biopolymer comprises at least one synthetic hydrogel or synthetic hydrogel made of amphiphilic copolymers consisting of units of ethylene oxide (PEO) and polypropylene oxide (PPO).
Embodiment 12 provides the system of embodiments 1-11, wherein the hydrogel is at about 2-50% w/v, about 3-40% w/v, about 4-30% w/v, about 5-25% w/v or about 10-20% w/v.
Embodiment 13 provides the system of embodiments 1-12, wherein the hydrogel is at about 28.6% w/v.
Embodiment 14 provides the system of embodiments 1-13, wherein the inner surface of the biocompatible construct comprises one or more extracellular matrix (ECM) components.
Embodiment 15 provides the system of embodiments 1-14, wherein the ECM component comprises collagen, laminin, fibronectin, hyaluronic acid, or a combination thereof.
Embodiment 16 provides the system of embodiments 1-15, wherein the ECM comprises collagen.
Embodiment 17 provides the system of embodiments 1-16, wherein the collagen is at a concentration of about 0.1-10 mg/ml or 0.1-9 mg/ml.
Embodiment 18 provides the system of embodiments 1-17, wherein the collagen is at a concentration of about 1 mg/ml.
Embodiment 19 provides the system of embodiments 1-18, wherein the ECM comprises laminin.
Embodiment 20 provides the system of embodiments 1-19, wherein the laminin is at a concentration of about 0.1-10 mg/ml or 0.1-9 mg/m.
Embodiment 21 provides the system of embodiments 1-20, wherein the laminin is at a concentration of about 1 mg/ml.
Embodiment 22 provides the system of embodiments 1-21, wherein the plurality of auditory neurons comprises at least 500 auditory neurons.
Embodiment 23 provides the system of embodiments 1-22, wherein at least one of the plurality of auditory neurons is in contact with the biocompatible construct.
Embodiment 24 provides the system of embodiments 1-23, wherein the at least one neuron in the plurality of neurons comprises a cell body and/or an axon.
Embodiment 25 provides the system of embodiments 1-24, wherein the axon is about 0.5-50 cm in length.
Embodiment 26 provides the system of embodiments 1-25, wherein a portion of the at least one neuron in the plurality of auditory neurons is positioned:
(i) at a first end of the biocompatible construct;
(ii) at a second end of the biocompatible construct;
(iii) at, along or within a portion of a body of the biocompatible construct; and/or
(iv) at, along, or within the entire body of the biocompatible construct.
Embodiment 27 provides the system of embodiments 1-26, wherein the portion of the at least one neuron in the plurality of auditory neuron positioned at the first end or at the second end of the biocompatible construct comprises a cell body.
Embodiment 28 provides the system of embodiments 1-27, wherein the portion of the at least one neuron in the plurality of auditory neuron positioned at, along or within a portion of the body of the biocompatible construct comprises an axon.
Embodiment 29 provides the system of embodiments 1-28, wherein the body of the biocompatible construct comprises an inner surface of the body.
Embodiment 30 provides the system of embodiments 1-29, wherein the inner surface comprises a luminal core.
Embodiment 31 provides the system of embodiments 1-30, wherein the portion of the at least one neuron in the plurality of auditory neuron positioned at, along or within the entire body of the biocompatible construct comprises an axon.
Embodiment 32 provides the system of embodiments 1-31, wherein the axon extends unidirectionally or bidirectionally.
Embodiment 33 provides the system of embodiments 1-32, wherein at least a portion of the plurality of auditory neurons comprises an aggregate of auditory neurons.
Embodiment 34 provides the system of embodiments 1-33, wherein the plurality of auditory neurons comprises:
(a) a spiral ganglion neuron, an inferior colliculus (IC) auditory neuron, a cortical auditory neuron, a type I auditory neuron or, a type II auditory neuron, or a combination thereof; or
(b) a neuron present in a cochlea, a spiral ganglion, an auditory nerve, an auditory brainstem nucleus, or a cortical brainstem nuclei of an organism, or a combination thereof
Embodiment 35 provides the system of embodiments 1-34, wherein the plurality of auditory neurons is or comprises a plurality of spiral ganglion neurons.
Embodiment 36 provides the system of embodiments 1-35, wherein the plurality of auditory neurons is or comprises a plurality of IC auditory neurons.
Embodiment 37 provides the system of embodiments 1-36, wherein the system further comprises one or more additional cells, such as astrocytes, oligodendrocytes, Schwann cells, microglia, or endothelial cells.
Embodiment 38 provides the system of embodiments 1-37, wherein the plurality of auditory neurons is isolated from a subject.
Embodiment 39 provides the system of embodiments 1-38, wherein the plurality of auditory neurons is or comprises at least one cell derived from a stem cell.
Embodiment 40 provides the system of embodiments 1-39, wherein the stem cell comprises one or more of an induced pluripotent stem cell (iPSC), a fetal stem cell, or a tissue stem cell.
Embodiment 41 provides the system of embodiments 1-40, wherein the plurality of auditory neurons is autologous, allogeneic, or xenogeneic to a subject.
Embodiment 42 provides the system of embodiments 1-41, wherein the at least one neuron in the plurality of auditory neurons is genetically engineered or reprogrammed using a non-genetic technique.
Embodiment 43 provides the system of embodiments 1-42, wherein the at least one genetically engineered neuron comprises a sensor, an actuator, a receptor and/or a reporter.
Embodiment 44 provides the system of embodiments 1-43, wherein the at least one genetically engineered neuron in the plurality of auditory neurons comprises a reporter.
Embodiment 45 provides the system of embodiments 1-44, wherein the reporter comprises a fluorescent reporter.
Embodiment 46 provides the system of embodiments 1-45, wherein the at least one genetically engineered neuron in the plurality of auditory neurons comprises a receptor.
Embodiment 47 provides the system of embodiments 1-46, wherein the receptor comprises one or more of a magnetoreceptor, an electromagnetic receptor, an electroreceptor, a hydroreceptor, a mechanoreceptor, an osmoreceptor, a thermoreceptor, and a piezoelectric ion channel.
Embodiment 48 provides the system of embodiments 1-47, wherein an electromagnetic receptor comprises one or more of an infrared receptor(s), a photoreceptor(s), or an ultraviolet receptor(s).
Embodiment 49 provides the system of embodiments 1-48, wherein the mechanoreceptor comprises one or both of a mechanosensory receptor, or a proprioreceptor.
Embodiment 50 provides the system of embodiments 1-49, wherein the at least one genetically engineered neuron in the plurality of auditory neurons comprises a sensor.
Embodiment 51 provides the system of embodiments 1-50, wherein the sensor comprises an optical sensor comprising one or more of a light-sensitive ion channel, a calcium sensor, and a membrane voltage sensor.
Embodiment 52 provides the system of embodiments 1-51, wherein the sensor comprises a calcium sensor comprising one or more of Aequorin, Cameleon, and GCaMP.
Embodiment 53 provides the system of embodiments 1-52, wherein the sensor comprises a chloride sensor comprising Clomeleon.
Embodiment 54 provides the system of embodiments 1-53, wherein the sensor comprises a membrane voltage sensor comprising Mermaid.
Embodiment 55 provides the system of embodiments 1-54, wherein the at least one genetically engineered neuron in the plurality of auditory neurons comprises an actuator.
Embodiment 56 provides the system of embodiments 1-55, wherein the actuator comprises an optical actuator comprising one or more of channelrhodopsin, halorhodopsin, and archaerhodopsin.
Embodiment 57 provides the system of embodiments 1-56, wherein the at least one genetically engineered neuron in the plurality of auditory neurons comprising an optical sensor and/or an optical actuator is an optogenetic neuron.
Embodiment 58 provides the system of embodiments 1-57, wherein the biocompatible construct is a tubular construct.
Embodiment 59 provides the system of embodiments 1-58, wherein the biocompatible construct is a spiral or a wye.
Embodiment 60 provides the system of embodiments 1-59, wherein the biocompatible construct has a curvature of about 240-900 degrees.
Embodiment 61 provides the system of embodiments 1-60, wherein the curvature of the biocompatible construct is about 270 degrees.
Embodiment 62 provides the system of embodiments 1-61, wherein the system has an outer diameter of about 0.1-40 mm.
Embodiment 63 provides the system of embodiments 1-62, wherein the system has the outer diameter of about 1.5 mm.
Embodiment 64 provides the system of embodiments 1-63, wherein the system has a diameter of about 0.5-70 mm or 0.1-10 mm.
Embodiment 65 provides the system of embodiments 1-64, wherein the system has the diameter of about 7 mm.
Embodiment 66 provides the system of embodiments 1-65, wherein the system has a pitch of about 0.1-10 mm.
Embodiment 67 provides the system of embodiments 1-66, wherein the system has the pitch of about 1 mm.
Embodiment 68 provides the system of embodiments 1-67, wherein the system is manufactured or assembled ex vivo or in vitro.
Embodiment 69 provides the system of embodiments 1-68, wherein the system is capable of or can be used to modulate at least one neuron in the plurality of neurons in the system and/or at least one neuron in a subject.
Embodiment 70 provides the system of embodiments 1-69, wherein modulating is or comprises unidirectional or bidirectional modulation.
Embodiment 71 provides the system of embodiments 1-70, wherein modulating comprises stimulating or inhibiting at least one neuron in the plurality of auditory neurons in the system or at least one neuron in a subject.
Embodiment 72 provides the system of embodiments 1-71, wherein stimulating comprises electrical stimulation, optical stimulation, sound wave/vibration stimulation, magnetic stimulation, or a combination thereof.
Embodiment 73 provides the system of embodiments 1-72, wherein stimulating comprises electrical stimulation and optical stimulation.
Embodiment 74 provides the system of embodiments 1-73, wherein modulating comprises monitoring and/or recording.
Embodiment 75 provides the system of embodiments 1-74, wherein the at least one neuron in the subject is or comprises an auditory neuron.
Embodiment 76 provides the system of embodiments 1-75, wherein an auditory neuron comprises:
(a) a spiral ganglion neuron, an IC neuron, or a combination thereof; or
(b) a neuron present in a cochlea, a spiral ganglion, an auditory nerve, an auditory brainstem nuclei, and/or cortical brainstem nuclei of an organism, or a combination thereof.
Embodiment 77 provides the system of embodiments 1-76, wherein the system is capable of stimulating or inhibiting an auditory pathway in a subject.
Embodiment 78 provides the system of embodiments 1-77, wherein the system is capable of stimulating or inhibiting a peripheral auditory pathway in a subject.
Embodiment 79 provides the system of embodiments 1-78, wherein the system is implanted at, near, or within a cochlea in a subject; at, near, or within an inferior colliculus (IC) in a subject; at, near or within a cochlear nucleus in a subject, and/or at, near or within an auditory cortex in a subject.
Embodiment 80 provides the system of embodiments 1-79, wherein the implanted system contacts at least one cell in a subject or at, near, or within areas of the auditory cortex in a subject.
Embodiment 81 provides the system of embodiments 1-80, wherein the at least one cell in a subject is an endogenous cell.
Embodiment 82 provides the system of embodiments 1-81, wherein the at least one cell comprises a neuron.
Embodiment 83 provides the system of embodiments 1-82, wherein the neuron comprises a neuron present in a cochlea, an auditory nerve, an auditory brainstem nuclei, a cortical brainstem nuclei, a spiral ganglion, an IC, or a combination thereof.
Embodiment 84 provides the system of embodiments 1-83, wherein contacting of the system with the at least one cell in the subject is determined to create a synapse between at least one neuron of the plurality of auditory neurons of the system and the at least one cell in the subject.
Embodiment 85 provides the system of embodiments 1-84, wherein a synapse is or comprises a neuronal synapse.
Embodiment 86 provides the system of embodiments 1-85, wherein the implanted system provides an accessible interface for modulating the at least one neuron in the plurality of auditory neurons in the system, or the at least one cell in the subject.
Embodiment 87 provides the system of embodiments 1-86, wherein the biocompatible construct has a non-linear shape.
Embodiment 88 provides a method for manufacturing a system comprising:
(a) providing a biocompatible construct comprising a first end, a second end, and a body; and
(b) associating the biocompatible construct with a plurality of auditory neurons.
Embodiment 89 provides the method of embodiment 88, wherein the method comprises maintaining the system under conditions that promotes growth of at least one neuron in the plurality of auditory neurons.
Embodiment 90 provides the method of embodiments 88-89, wherein the method comprises maintaining the system under conditions that maintain viability of the at least one neuron in the plurality of auditory neurons.
Embodiment 91 provides the method of embodiments 88-90, wherein the method comprises forming an aggregate of at least a portion of the plurality of auditory neurons.
Embodiment 92 provides the method of embodiments 88-91, wherein the aggregate of at least a portion of the plurality of auditory neurons is formed prior to contacting the plurality of auditory neurons with the biocompatible construct.
Embodiment 93 provides the method of embodiments 88-92, wherein the system comprises a system provided in any one of embodiments 1-87.
Embodiment 94 provides a method for implanting in a subject, a system comprising:
(a) a biocompatible construct comprising a first end, a second end, and a body; and
(b) a plurality of auditory neurons,
the method comprising implanting the system at, near, or within a cochlea, a cochlear nucleus, an auditory cortex, or an inferior colliculus (IC) in a subject.
Embodiment 95 provides the method of embodiment 94, wherein the implantation is determined to be less likely to cause inflammation compared to implantation of conventional microelectrodes.
Embodiment 96 provides the method of embodiments 94-95, wherein the subject is a human.
Embodiment 97 provides the method of embodiments 94-96, wherein the system comprises a system provided in any one of embodiments 1-87.
Embodiment 98 provides a method of modulating at least one neuron in a subject, comprising:
(i) providing to the subject, a system comprising: (a) a biocompatible construct comprising a first end, a second end, and a body; and (b) a plurality of auditory neurons, and
(ii) modulating at least one neuron with electrical stimulation, optical stimulation or both.
Embodiment 99 provides the method of embodiment 98, wherein modulating comprises monitoring, recording, stimulating and/or inhibiting.
Embodiment 100 provides the method of embodiments 98-99, wherein the at least one neuron in the subject comprises an auditory neuron.
Embodiment 101 provides the method of embodiments 98-100, wherein the auditory neuron comprises one or more of a spiral ganglion neuron, or an IC neuron.
Embodiment 102 provides the method of embodiments 98-101, wherein the subject is a human.
Embodiment 103 provides the method of embodiments 98-102, wherein the system comprises a system provided in any one of embodiments 1-87.
Embodiment 104 provides a method of treating a subject having a hearing loss disorder, comprising providing to the subject a system comprising: (a) a biocompatible construct comprising a first end, a second end, and a body; and (b) a plurality of auditory neurons, thereby treating the subject.
Embodiment 105 provides the method of embodiment 104, wherein the hearing loss disorder comprises one or more of conducting hearing loss, sensorineural hearing loss or mixed hearing loss.
Embodiment 106 provides the method of embodiments 104-105, wherein the hearing loss disorder comprises one or more of acquired hearing loss disorder, sudden hearing loss disorder, secondary hearing loss (drug induced or injury induce hearing loss), autoimmune inner ear disease, otosclerosis, Meniere's disease, presbycusis, or acoustic neuroma.
Embodiment 106 provides the method of embodiments 104-106, wherein the subject is a human.
Embodiment 108 provides the method of embodiments 104-107, wherein the system comprises a system provided in any one of embodiments 1-87.
Embodiment 109 provides a method of ameliorating a symptom of a hearing loss disorder in a subject, comprising providing to the subject a system comprising: (a) a biocompatible construct comprising a first end, a second end, and a body; and (b) a plurality of auditory neurons.
Embodiment 110 provides the method of embodiment 109, wherein the hearing loss disorder comprises one or more of conducting hearing loss, sensorineural hearing loss or mixed hearing loss.
Embodiment 111 provides the method of embodiments 109-110, wherein the hearing loss disorder comprises one or more of acquired hearing loss disorder, sudden hearing loss disorder, secondary hearing loss (drug induced or injury induce hearing loss), autoimmune inner ear disease, otosclerosis, Meniere's disease, presbycusis, or acoustic neuroma.
Embodiment 112 provides the method of embodiments 109-111, wherein the subject is a human.
Embodiment 113 provides the method of embodiments 109-112, wherein the system comprises a system provided in any one of embodiments 1-87.
Embodiment 114 provides a method of preventing the worsening of a symptom of a hearing loss disorder in a subject, comprising providing to the subject a system comprising: (a) a biocompatible construct comprising a first end, a second end, and a body; and (b) a plurality of auditory neurons.
Embodiment 115 provides the method of embodiment 114, wherein the hearing loss disorder comprises one or more of conducting hearing loss, sensorineural hearing loss or mixed hearing loss.
Embodiment 116 provides the method of embodiments 114-115, wherein a hearing loss disorder comprises one or more of acquired hearing loss disorder, sudden hearing loss disorder, secondary hearing loss (drug induced or injury induce hearing loss), autoimmune inner ear disease, otosclerosis, Meniere's disease, presbycusis, or acoustic neuroma.
Embodiment 117 provides the method of embodiments 114-116, wherein the subject is a human.
Embodiment 118 provides the method of embodiments 114-117, wherein the system comprises the system provided in any one of embodiments 1-87.
Embodiment 119 provides a kit comprising a system comprising:
(a) a biocompatible construct comprising a first end, a second end, and a body; and
(b) a plurality of auditory neurons, and instructions for using the same.
Embodiment 120 provides the kit of embodiment 119, wherein the further comprises instructions for using the same.
Embodiment 121 provides the kit of embodiment 119-120, wherein the system comprises a system provided in any one of embodiments 1-87.
Embodiment 122 provides a cochlear implant comprising:
a stimulator array;
a plurality of living electrodes, wherein at least one of the plurality of living electrodes is in optical communication with an element of the stimulator array, each of the plurality of living electrodes comprising:
   a proximal end comprising stimulator-complementary neurons; and
   a distal end comprising auditory neurons in neurologic communication with optogenetic neurons within the living electrode;
      wherein the plurality of living electrodes have staggered distal ends.
Embodiment 123 provides a cochlear implant of embodiment 122, wherein:
the stimulator array is an optrode array; and
the stimulator-complementary neurons are optogenetic neurons.

### EQUIVALENTS

Those skilled in the art will recognize, or be able to ascertain using no more than routine experimentation, many equivalents to the specific embodiments of the invention described herein. It is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Further, it should also be understood that any embodiment or aspect of the invention can be explicitly excluded from the claims, regardless of whether the specific exclusion is recited in the specification. The scope of the present invention is not intended to be limited to the above Description, but rather is as set forth in the claims that follow.

1. A system comprising:
(a) a biocompatible construct comprising a first end, a second end, and a body; and
(b) a plurality of auditory neurons.

2. The system of claim 1, wherein the biocompatible construct comprises an inner surface and an outer surface.

3. The system of claim 2, wherein the inner surface of the biocompatible construct defines a luminal core.

4. The system of claim 2, wherein the outer surface of the biocompatible construct comprises at least one hydrogel.

5. The system of claim 4, wherein the hydrogel comprises a hydrophilic biopolymer and/or a synthetic polymer.

6. The system of claim 4, wherein the hydrogel is at least partially cross-linked, wherein the cross-linking optionally increases stiffness, reduces porosity, and/or increases degradation time.

7. The system of claim 5, wherein the hydrophillic biopolymer comprises one or more of agarose, hydrogel, hyaluronan, chitosan, alginate, collagen, dextran, pectin, carrageenan, polylysine, gelatin, hyaluronic acid, fibrin, and methylcellulose.

8. The system of claim 7, wherein the hydrophillic biopolymer comprises agarose.

9. The system of claim 7, wherein the agarose is at about 0.25-30%, about 0.25%-3%, about 0.5%-3%, about 1-20%, about 1.5-10%, about 2-9%, about 2.5-8%, or about 3-7%.

10. The system of claim 9, wherein the agarose is at about 3%.

11. The system of claim 7, wherein the hydrophilic biopolymer comprises at least one synthetic hydrogel or synthetic hydrogel made of amphiphilic copolymers consisting of units of ethylene oxide (PEO) and polypropylene oxide (PPO).

12. The system of claim 7, wherein the hydrogel is at about 2-50% w/v, about 3-40% w/v, about 4-30% w/v, about 5-25% w/v or about 10-20% w/v.

13. The system of claim 12, wherein the hydrogel is at about 28.6% w/v.

14. The system of claim 1, wherein the inner surface of the biocompatible construct comprises one or more extracellular matrix (ECM) components.

15. The system of claim 14, wherein the ECM component comprises collagen, laminin, fibronectin, hyaluronic acid, or a combination thereof.

16. The system of claim 15, wherein the ECM comprises collagen.

17. The system of claim 16, wherein the collagen is at a concentration of about 0.1-10 mg/ml or 0.1-9 mg/ml.

18. The system of claim 16, wherein the collagen is at a concentration of about 1 mg/ml.

19. The system of claim 15, wherein the ECM comprises laminin.

20. The system of claim 19, wherein the laminin is at a concentration of about 0.1-10 mg/ml or 0.1-9 mg/ml.

21. The system of claim 19, wherein the laminin is at a concentration of about 1 mg/ml.

22. The system of claim 1, wherein the plurality of auditory neurons comprises at least 500 auditory neurons.

23. The system of claim 1, wherein at least one of the plurality of auditory neurons is in contact with the biocompatible construct.

24. The system of claim 1, wherein the at least one neuron in the plurality of neurons comprises a cell body and/or an axon.

25. The system of claim 24, wherein the axon is about 0.5-50 cm in length.

26. The system of claim 1, wherein a portion of the at least one neuron in the plurality of auditory neurons is positioned:
(i) at a first end of the biocompatible construct;
(ii) at a second end of the biocompatible construct;
(iii) at, along or within a portion of a body of the biocompatible construct; and/or
(iv) at, along, or within the entire body of the biocompatible construct.

27. The system of claim 26, wherein the portion of the at least one neuron in the plurality of auditory neuron positioned at the first end or at the second end of the biocompatible construct comprises a cell body.

28. The system of claim 26, wherein the portion of the at least one neuron in the plurality of auditory neuron positioned at, along or within a portion of the body of the biocompatible construct comprises an axon.

29. The system of claim 26 (iii) or (iv), wherein the body of the biocompatible construct comprises an inner surface of the body.

30. The system of claim 29, wherein the inner surface comprises a luminal core.

31. The system of claim 26 (iii) or (iv) or claim 30, wherein the portion of the at least one neuron in the plurality of auditory neuron positioned at, along or within the entire body of the biocompatible construct comprises an axon.

32. The system of claim 31, wherein the axon extends unidirectionally or bidirectionally.

33. The system of claim 1, wherein at least a portion of the plurality of auditory neurons comprises an aggregate of auditory neurons.

34. The system of claim 1, wherein the plurality of auditory neurons comprises:
(a) a spiral ganglion neuron, an inferior colliculus (IC) auditory neuron, a cortical auditory neuron, a type I auditory neuron or a type II auditory neuron, or a combination thereof; or
(b) a neuron present in a cochlea, a spiral ganglion, an auditory nerve, an auditory brainstem nucleus, or a cortical brainstem nuclei of an organism, or a combination thereof.

35. The system of claim 34, wherein the plurality of auditory neurons is or comprises a plurality of spiral ganglion neurons.

36. The system of claim 34, wherein the plurality of auditory neurons is or comprises a plurality of IC auditory neurons.

37. The system of claim 1, wherein the system further comprises one or more additional cells, such as astrocytes, oligodendrocytes, Schwann cells, microglia, or endothelial cells.

38. The system of claim 1, wherein the plurality of auditory neurons is isolated from a subject.

39. The system of claim 1, wherein the plurality of auditory neurons is or comprises at least one cell derived from a stem cell.

40. The system of claim 39, wherein the stem cell comprises one or more of an induced pluripotent stem cell (iPSC), a fetal stem cell, or a tissue stem cell.

41. The system of claim 1, wherein the plurality of auditory neurons is autologous, allogeneic, or xenogeneic to the subject.

42. The system of claim 1, wherein the at least one neuron in the plurality of auditory neurons is genetically engineered or reprogrammed using a non-genetic technique.

43. The system of claim 42, wherein the at least one genetically engineered neuron comprises a sensor, an actuator, a receptor and/or a reporter.

44. The system of claim 43, wherein the at least one genetically engineered neuron in the plurality of auditory neurons comprises a reporter.

45. The system of claim 44, wherein the reporter comprises a fluorescent reporter.

46. The system of claim 43, wherein the at least one genetically engineered neuron in the plurality of auditory neurons comprises a receptor.

47. The system of claim 46, wherein the receptor comprises one or more of a magnetoreceptor, an electromagnetic receptor, an electroreceptor, a hydroreceptor, a mechanoreceptor, an osmoreceptor, a thermoreceptor, and a piezoelectric ion channel.

48. The system of claim 47 wherein an electromagnetic receptor comprises one or more of an infrared receptor(s), a photoreceptor(s), or an ultraviolet receptor(s).

49. The system of claim 47, wherein the mechanoreceptor comprises one or both of a mechanosensory receptor, or a proprioreceptor.

50. The system of claim 43, wherein the at least one genetically engineered neuron in the plurality of auditory neurons comprises a sensor.

51. The system of claim 50, wherein the sensor comprises an optical sensor comprising one or more of a light-sensitive ion channel, a calcium sensor, and a membrane voltage sensor.

52. The system of claim 50, wherein the sensor comprises a calcium sensor comprising one or more of Aequorin, Cameleon, and GCaMP.

53. The system of claim 50, wherein the sensor comprises a chloride sensor comprising Clomeleon.

54. The system of claim 50, wherein the sensor comprises a membrane voltage sensor comprising Mermaid.

55. The system of claim 43, wherein the at least one genetically engineered neuron in the plurality of auditory neurons comprises an actuator.

56. The system of claim 55, wherein the actuator comprises an optical actuator comprising one or more of channelrhodopsin, halorhodopsin, and archaerhodopsin.

57. The system of claim 51, wherein the at least one genetically engineered neuron in the plurality of auditory neurons comprising an optical sensor and/or an optical actuator is an optogenetic neuron.

58. The system of claim 1, wherein the biocompatible construct is a tubular construct.

59. The system of claim 1, wherein the biocompatible construct is a spiral or a wye.

60. The system of claim 1, wherein the biocompatible construct has a curvature of about 240-900 degrees.

61. The system of claim 60, wherein the curvature of the biocompatible construct is about 270 degrees.

62. The system of claim 60, wherein the system has an outer diameter of about 0.1-40 mm.

63. The system of claim 62, wherein the system has the outer diameter of about 1.5 mm.

64. The system of claim 60, wherein the system has a diameter of about 0.5-70 mm or 0.1-10 mm.

65. The system of claim 64, wherein the system has the diameter of about 7 mm.

66. The system of claim 60, wherein the system has a pitch of about 0.1-10 mm.

67. The system of claim 66, wherein the system has the pitch of about 1 mm.

68. The system of claim 1, wherein the system is manufactured or assembled ex vivo or in vitro.

69. The system of claim 1, wherein the system is capable of or can be used to modulate at least one neuron in the plurality of neurons in the system and/or at least one neuron in a subject.

70. The system of claim 69, wherein modulating is or comprises unidirectional or bidirectional modulation.

71. The system of claim 69, wherein modulating comprises stimulating or inhibiting at least one neuron in the plurality of auditory neurons in the system or at least one neuron in a subject.

72. The system of claim 71, wherein stimulating comprises electrical stimulation, optical stimulation, sound wave/vibration stimulation, magnetic stimulation, or a combination thereof.

73. The system of claim 71, wherein stimulating comprises electrical stimulation and optical stimulation.

74. The system of claim 69, wherein modulating comprises monitoring and/or recording.

75. The system of claim 69, wherein the at least one neuron in the subject is or comprises an auditory neuron.

76. The system of claim 75, wherein an auditory neuron comprises:
(a) a spiral ganglion neuron, an IC neuron, or a combination thereof; or
(b) a neuron present in a cochlea, a spiral ganglion, an auditory nerve, an auditory brainstem nuclei, and/or cortical brainstem nuclei of an organism, or a combination thereof.

77. The system of claim 1, wherein the system is capable of stimulating or inhibiting an auditory pathway in a subject.

78. The system of claim 1, wherein the system is capable of stimulating or inhibiting a peripheral auditory pathway in a subject.

79. The system of claim 1, wherein the system is implanted at, near, or within a cochlea in a subject; at, near, or within an inferior colliculus (IC) in a subject; at, near or within a cochlear nucleus in a subject, and/or at, near or within an auditory cortex in a subject.

80. The system of claim 79, wherein the implanted system contacts at least one cell in a subject or at, near, or within areas of the auditory cortex in a subject.

81. The system of claim 80, wherein the at least one cell in the subject is an endogenous cell.

82. The system of claim 81, wherein the at least one cell comprises a neuron.

83. The system of claim 82, wherein the neuron comprises a neuron present in a cochlea, an auditory nerve, an auditory brainstem nuclei, a cortical brainstem nuclei, a spiral ganglion, an IC, or a combination thereof.

84. The system of claim 80, wherein contacting of the system with the at least one cell in the subject is determined to create a synapse between at least one neuron of the plurality of auditory neurons of the system and the at least one cell in the subject.

85. The system of claim 84, wherein a synapse is or comprises a neuronal synapse.

86. The system of claim 79, wherein the implanted system provides an accessible interface for modulating the at least one neuron in the plurality of auditory neurons in the system, or the at least one cell in the subject.

87. The system of claim 1, wherein the biocompatible construct has a non-linear shape.

88. A method for manufacturing a system comprising:
(a) providing a biocompatible construct comprising a first end, a second end, and a body; and
(b) associating the biocompatible construct with a plurality of auditory neurons.

89. The method of claim 88, wherein the method comprises maintaining the system under conditions that promote growth of at least one neuron in the plurality of auditory neurons.

90. The method of claim 88, wherein the method comprises maintaining the system under conditions that maintain viability of the at least one neuron in the plurality of auditory neurons.

91. The method of claim 88, wherein the method comprises forming an aggregate of at least a portion of the plurality of auditory neurons.

92. The method of claim 91, wherein the aggregate of at least a portion of the plurality of auditory neurons is formed prior to contacting the plurality of auditory neurons with the biocompatible construct.

93. (canceled)

94. A method for implanting in a subject, a system comprising:
(a) a biocompatible construct comprising a first end, a second end, and a body; and
(b) a plurality of auditory neurons,
the method comprising implanting the system at, near, or within a cochlea, a cochlear nucleus, an auditory cortex, or an inferior colliculus (IC) in a subject.

95. The method of claim 94, wherein the implantation is determined to be less likely to cause inflammation compared to implantation of conventional microelectrodes.

96. The method of claim 94, wherein the subject is a human.

97. (canceled)

98. A method of modulating at least one neuron in a subject, comprising:
(i) providing to the subject, a system comprising: (a) a biocompatible construct comprising a first end, a second end, and a body; and (b) a plurality of auditory neurons, and
(ii) modulating at least one neuron with electrical stimulation, optical stimulation or both.

99. The method of claim 98, wherein modulating comprises monitoring, recording, stimulating and/or inhibiting.

100. The method of claim 98, wherein the at least one neuron in the subject comprises an auditory neuron.

101. The method of claim 100, wherein the auditory neuron comprises one or more of a spiral ganglion neuron, or an IC neuron.

102. The method of claim 98, wherein the subject is a human.

103. (canceled)

104. A method of treating a subject having a hearing loss disorder, comprising providing to the subject a system comprising: (a) a biocompatible construct comprising a first end, a second end, and a body; and (b) a plurality of auditory neurons, thereby treating the subject.

105. The method of claim 104, wherein the hearing loss disorder comprises one or more of conducting hearing loss, sensorineural hearing loss or mixed hearing loss.

106. The method of claim 104, wherein the hearing loss disorder comprises one or more of acquired hearing loss disorder, sudden hearing loss disorder, secondary hearing loss (drug induced or injury induce hearing loss), autoimmune inner ear disease, otosclerosis, Meniere's disease, presbycusis, or acoustic neuroma.

107. The method of claim 104, wherein the subject is a human.

108. (canceled)

109. A method of ameliorating a symptom of a hearing loss disorder in a subject, comprising providing to the subject a system comprising: (a) a biocompatible construct comprising a first end, a second end, and a body; and (b) a plurality of auditory neurons.

110. The method of claim 109, wherein the hearing loss disorder comprises one or more of conducting hearing loss, sensorineural hearing loss or mixed hearing loss.

111. The method of claim 109, wherein the hearing loss disorder comprises one or more of acquired hearing loss disorder, sudden hearing loss disorder, secondary hearing loss (drug induced or injury induce hearing loss), autoimmune inner ear disease, otosclerosis, Meniere's disease, presbycusis, or acoustic neuroma.

112. The method of claim 109, wherein the subject is a human.

113. (canceled)

114. A method of preventing the worsening of a symptom of a hearing loss disorder in a subject, comprising providing to the subject a system comprising: (a) a biocompatible construct comprising a first end, a second end, and a body; and (b) a plurality of auditory neurons.

115. The method of claim 114, wherein the hearing loss disorder comprises one or more of conducting hearing loss, sensorineural hearing loss or mixed hearing loss.

116. The method of claim 114, wherein the hearing loss disorder comprises one or more of acquired hearing loss disorder, sudden hearing loss disorder, secondary hearing loss (drug induced or injury induce hearing loss), autoimmune inner ear disease, otosclerosis, Meniere's disease, presbycusis, or acoustic neuroma.

117. The method of claim 114, wherein the subject is a human.

118. (canceled)

119. A kit comprising a system comprising:
(a) a biocompatible construct comprising a first end, a second end, and a body; and
(b) a plurality of auditory neurons, and instructions for using the same.

120. The kit of claim 119, further comprising instructions for using the same.

121. (canceled)

122. A cochlear implant comprising:
a stimulator array;
a plurality of living electrodes, wherein at least one of the plurality of living electrodes is in optical communication with an element of the stimulator array, each of the plurality of living electrodes comprising:
a proximal end comprising stimulator-complementary neurons; and
a distal end comprising auditory neurons in neurologic communication with optogenetic neurons within the living electrode;
wherein the plurality of living electrodes have staggered distal ends.

123. The cochlear implant of claim 122, wherein:
the stimulator array is an optrode array; and
the stimulator-complementary neurons are optogenetic neurons.
